(19) United States
(12) Patent Application Publication
(10) Pub. No.: US 2025/0289897 A1
(43) Pub. Date: Sep. 18, 2025

(54) LEPTIN RECEPTOR AGONISTIC ANTIBODY AS MONO OR COMBO THERAPIES TO TREAT OBESITY

(71) Applicant: ELI LILLY AND COMPANY, Indianapolis, IN (US)

(72) Inventors: Minrong AI, Zionsville, IN (US); Todd C. CHILTON, San Diego, CA (US); Daniel A. BRIERE, Morgantown, IN (US); Paul F. CAIN, Bargersville, IN (US)

(21) Appl. No.: 19/078,529

(22) Filed: Mar. 13, 2025

Related U.S. Application Data
(60) Provisional application No. 63/566,017, filed on Mar. 15, 2024.

(51) Int. Cl. C07K 16/28; A61K 38/26; A61K 39/00; A61P 3/04

(52) U.S. Cl. CPC C07K 16/2869; A61K 38/26; A61P 3/04; A61K 2039/505

(57) ABSTRACT

Leptin receptor (LEPR) is a single-pass transmembrane receptor that belongs to the cytokine receptor family. The secreted ligand Leptin binds to LEPR and activates intracellular downstream signal cascades. Leptin and LEPR play critical role in body weight regulation. The antibodies of the present disclosure have agonistic activities on LEPR and can therefore be used as therapeutic agents to treat obesity.

### FIELD

The present disclosure relates to leptin receptor agonist antibodies and therapies related thereto.

### REFERENCE TO SEQUENCE LISTING

The present disclosure includes a Sequence Listing in ST.26 XML format. The Sequence Listing is provided as a file titled “30660_US_PRI Sequence Listing.xml” created Mar. 19, 2025, and is 45 kilobytes in size. The Sequence Listing information in the ST.26 XML format is incorporated herein by reference in its entirety.

### BACKGROUND

Leptin is a circulating hormone produced and released by adipocytes. Leptin signals through its receptor (LEPR, also termed OB-R) expressed by neurons in the hypothalamus to regulate metabolism and appetite. LEPR is a cell surface single-pass transmembrane receptor of the class I cytokine receptor family. Activation of LEPR by leptin binding activates intracellular Janus kinase (JAK) leading to phosphorylation of downstream effector proteins.

Loss-of-function mutations in leptin or LEPR genes cause severe early childhood onset of obesity. Recombinant leptin protein (metreleptin) was approved to treat symptoms in leptin deficient patients and in lipodystrophy patients. Metreleptin treatment was also shown to reduce body weight in humans with low circulating leptin (Depaoli et al., Diabetes. 2018 Jul 67:296-LB) and improve liver pathology in non-alcoholic steatohepatitis patients (Akinci et al. Med. 2021 Jul. 9;2 (7): 814-835). However, due to its short half-life, adverse side effects and the development of anti-leptin auto-antibodies, metreleptin has limited application in the clinic. In addition, patients with common obesity do not respond to metreleptin treatment due to leptin resistance. (Farr et al. Curr Opinion Endo Diab Obes. 2015; 22 (5): 353-359).

Additionally, LEPR antibodies have been reported in WO2017066204A1 and WO2019195796A1. However, there remains a need for LEPR agonist antibodies that bind LEPR of several species with desirable binding affinities, activate LEPR of several species, display beneficial properties with regard to binding of Fcγ receptors, do not desensitize LEPR signaling, activate signaling deficient LEPR, and/or can be advantageously combined with a secondary therapeutic agent.

Therefore, a new therapeutic agent to agonize the LEPR pathway will significantly broaden the clinical applications.

### SUMMARY

The disclosure provides antibodies that bind and agonize the leptin receptor (LEPR).

In an embodiment, antibodies of the disclosure include at least one of HCDR1, HCDR2, HCDR3, LCDR1, LCDR2, and LCDR3 selected from at least one of SEQ ID NO: 1, 2, 3, 4, 5, 6, 7, 8, and 9, or sequences that have at least 90%, at least 95%, at least 97%, or at least 99% sequence identity thereto.

In an embodiment, antibodies include an HCDR1 having SEQ ID NO:7, an HCDR2 having SEQ ID NO:8, an HCDR3 having SEQ ID NO:9, an LCDR1 having SEQ ID NO:1 or 2, an LCDR2 having SEQ ID NO:3, and an LCDR3 having SEQ ID NO:4, 5, or 6.

In one embodiment, an antibody includes LCDR1 having SEQ ID NO:1 and the LCDR3 having SEQ ID NO:5. In one embodiment, an antibody includes LCDR1 having SEQ ID NO:1 and the LCDR3 having SEQ ID NO:4. In one embodiment, an antibody includes LCDR1 having SEQ ID NO:2 and the LCDR3 having SEQ ID NO:5. In one embodiment, an antibody includes LCDR1 having SEQ ID NO:1 and the LCDR3 having SEQ ID NO:6.

In an embodiment, an antibody includes VH and VL sequences selected from SEQ ID NO: 10, 12, 14, 16, and 18, or sequences that have at least 90%, at least 95%, at least 97%, or at least 99% sequence identity thereto.

In one embodiment, the disclosure provides an antibody having a VH as depicted in SEQ ID NO:18 and a VL as depicted in SEQ ID NO:10. In one embodiment, the disclosure provides an antibody having a VH as depicted in SEQ ID NO:18 and a VL as depicted in SEQ ID NO:12. In one embodiment, the disclosure provides an antibody having a VH as depicted in SEQ ID NO: 18 and a VL as depicted in SEQ ID NO:14. In one embodiment, the disclosure provides an antibody having a VH as depicted in SEQ ID NO:18 and a VL as depicted in SEQ ID NO:16.

In some embodiments, an antibody or antigen binding fragment disclosed herein are human IgG1 subclass or IgG4 subclass. In some embodiments, an antibody or antigen binding fragment disclosed herein are human IgG1 subclass.

In an embodiment, an antibody includes HC and LC sequences selected from SEQ ID NO: 11, 13, 15, 17, and 19, or sequences that have at least 90%, at least 95%, at least 97%, or at least 99% sequence identity thereto.

In one embodiment, the disclosure provides an antibody having an HC as depicted in SEQ ID NO:19 and an LC as depicted in SEQ ID NO:11. In one embodiment, the disclosure provides an antibody having an HC as depicted in SEQ ID NO:19 and an LC as depicted in SEQ ID NO:13. In one embodiment, the disclosure provides an antibody having an HC as depicted in SEQ ID NO:19 and an LC as depicted in SEQ ID NO:15. In one embodiment, the disclosure provides an antibody having an HC as depicted in SEQ ID NO:19 and an LC as depicted in SEQ ID NO:17.

In another aspect, the disclosure provides a pharmaceutical composition including an antibody of the present disclosure. In one embodiment, the pharmaceutical composition includes antibody of the present disclosure and a second therapeutic agent. The second therapeutic agent is selected from an agent that has GLP-1 receptor agonist activity, an agent that has amylin receptors agonist activity, an agent that has calcitonin receptor agonist activity, an agent that has glucagon receptor agonist activity. In an embodiment, the second therapeutic agent has GLP-1 receptor agonist activity and GIP receptor agonist activity. In another embodiment, the second therapeutic agent has GLP-1 receptor agonist activity, GIP receptor agonist activity, and glucagon receptor agonist activity.

In another aspect, the disclosure provides a method of treating a disease or disorder that is associated with the leptin receptor. In one embodiment, a disease or disorder is associated with or mediated by leptin deficiency, leptin resistance, or otherwise treatable by agonizing LEPR. In another embodiment, the disease or disorder includes obesity. The method according to this aspect includes administering an antibody of the disclosure as a monotherapy or as a combination therapy.

In the case of a combination therapy, an antibody of the present disclosure is administered simultaneously, separately, or sequentially in combination with a second therapeutic agent. The second therapeutic agent includes an agent that has GLP-1 receptor agonist activity, amylin receptor agonist activity, or glucagon receptor agonist activity. In an embodiment, the second therapeutic has GLP-1 receptor agonist activity and GIP receptor agonist activity. In another embodiment, the second therapeutic agent has GLP-1 receptor agonist activity, GIP receptor agonist activity, and glucagon receptor agonist activity.

In some embodiments, the second therapeutic agent is selected from SEQ ID NO:39-57.

### BRIEF DESCRIPTION OF THE DRAWINGS

FIG. 1 depicts binding of exemplary antibodies of the disclosure to human LEPR.

FIG. 2 depicts binding of exemplary antibodies of the disclosure to hLepR, in the presence of leptin protein, as a percentage of control.

FIG. 3 depicts C1q binding of an exemplary antibody of the disclosure.

FIG. 4 depicts percent body weight, as compared to control, of diet induced obese mice that were administered GLP-1 receptor agonist in combination with leptin receptor agonist.

FIG. 5 depicts percent body weight, as compared to control, of diet induced obese mice that were administered a dual GLP1R/GIPR agonist in combination with leptin receptor agonist.

FIG. 6 depicts percent body weight, as compared to control, of diet induced obese mice that were administered a GLP1R/GIPR/GCGR triple agonist in combination with leptin receptor agonist.

FIG. 7 depicts percent body weight, as compared to control, of diet induced obese mice that were administered a glucagon receptor agonist in combination with leptin receptor agonist.

FIG. 8 depicts percent body weight, as compared to control, of diet induced obese mice that were administered an amylin receptor agonist in combination with leptin receptor agonist.

FIG. 9 depicts percent body weight, as compared to control, of diet induced obese mice that were administered a leptin receptor agonist in combination with either amylin receptor agonist or amylin and calcitonin receptor co-agonist.

FIG. 10 depicts percent body weight, as compared to control, of cynomolgus monkeys that were administered an exemplary antibody disclosed herein.

### DETAILED DESCRIPTION

The present disclosure provides antibodies that bind and agonize the leptin receptor (LEPR). As used herein and unless otherwise noted, LEPR refers to the human leptin receptor (hLepR), as depicted in SEQ ID NO:28. The extracellular domain of human LEPR is depicted in SEQ ID NO:29.

The term “antibody” as used herein, refers to an immunoglobulin molecule that binds an antigen. Embodiments of an antibody include a monoclonal antibody, polyclonal antibody, single chain fragment variable (scFv), antibody fragment, human antibody, humanized antibody, chimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4).

An exemplary antibody of the present disclosure is an Immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function and antibody recycling. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

In some embodiments, an antibody or antigen binding fragment disclosed herein are human IgG1 subclass or IgG4 subclass.

In some embodiments, an antibody or antigen binding fragment disclosed herein are human IgG1 subclass.

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, HCDR3” and the three CDRs of the light chain are referred to as “LCDR1, LCDR2, and LCDR3.” The CDRs contain most of the residues that form specific interactions with the antigen. Assignment of amino acid residues to the CDRs may be done according to well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology 1987, 196:901-917; Al-Lazikani et al., “Standard conformations for the canonical structures of immunoglobulins”, Journal of Molecular Biology 1997, 273:927-948), North (North et al., “A New Clustering of Antibody CDR Loop Conformations”, Journal of Molecular Biology, 406, 228-256 (2011), or IMGT (the international ImMunoGeneTics database available on at www.imgt.org; see Lefranc et al., Nucleic Acids Res. 1999; 27:209-212). Unless otherwise noted, the North CDR definitions are used for the antibodies that bind human LEPR described herein.

Embodiments of the present disclosure also include antibody fragments or antigen-binding fragments that, as used herein, comprise at least a portion of an antibody retaining the ability to specifically interact with an antigen or an epitope of the antigen, such as Fab, Fab′, F (ab′)2, Fv fragments, scFv antibody fragments, scFab, disulfide-linked Fvs (sdFv), or a Fd fragment.

The term “antigen binding domain”, as used herein, refers to a portion of a molecule that binds an antigen or an epitope of the antigen.

The term “bispecific”, as used herein, refers to a molecule that comprises two distinct antigen-binding domains. A bispecific binding molecule can bind two different antigens or two different epitopes of the same antigen. Embodiments of the present disclosure also include a bispecific antibody that bind to LEPR.

The disclosure provides a VH including heavy chain complementarity determining regions HCDR1, HCDR2, and HCDR3, which, in turn comprise a heavy chain (HC).

In some embodiments, the disclosure provides a VH including at least one of SEQ ID NO: 7, SEQ ID NO:8, and SEQ ID NO:9.

In some embodiments, an antibody of the disclosure provides a VH including at least one of HCDR1 (SEQ ID NO:7), HCDR2 (SEQ ID NO:8), and HCDR3 (SEQ ID NO:9). An exemplary VH includes a sequence being at least 75%, at least 80%, at least 85%, at least 90%, at least 95%, at least 97%, or at least 99% identical to an amino acid sequence as depicted in SEQ ID NO:18.

An exemplary HC having HCDR1 (SEQ ID NO:7), HCDR2 (SEQ ID NO:8), and HCDR3 (SEQ ID NO:9) includes a sequence being at least 75%, at least 80%, at least 85%, at least 90%, at least 95%, at least 97%, or at least 99% identical to an amino acid sequence as depicted in SEQ ID NO:19.

In some embodiments, the disclosure provides an antibody having LCDR 1 as depicted in SEQ ID NO:1 and LCDR3 as depicted in SEQ ID NO:5. In some embodiments, the antibody further includes an LCDR2 as depicted in SEQ ID NO:3.

In some embodiments, the disclosure provides a VL that includes SEQ ID NO:1, SEQ ID NO: 3 and SEQ ID NO:5. An exemplary VL is depicted in SEQ ID NO:16. In some embodiments, the VL includes a sequence being at least 75%, at least 80%, at least 85%, 90%, at least 95%, at least 97%, or at least 99% identical to an amino acid sequence as depicted in SEQ ID NO:16.

An exemplary LC having SEQ ID NO:1, SEQ ID NO:3, and SEQ ID NO:5 includes a sequence being at least 75%, at least 80%, at least 85%, at least 90%, at least 95%, at least 97%, or at least 99% identical to an amino acid sequence as depicted in SEQ ID NO:17.

In one embodiment, an antibody of the disclosure comprises a heavy chain (HC) comprising SEQ ID NO: 19, and a light chain (LC) comprising SEQ ID NO: 17.

In one embodiment, an antibody of the disclosure comprises a heavy chain (HC) consisting of SEQ ID NO:19, and a light chain (LC) consisting of SEQ ID NO:17.

In some embodiments, the disclosure provides an antibody having SEQ ID NO:1 and SEQ ID NO:4. In some embodiments, the antibody further includes an LCDR2 as depicted in SEQ ID NO:3.

In some embodiments, the disclosure provides a VL that includes SEQ ID NO:1, SEQ ID NO: 3, and SEQ ID NO:4. An exemplary VL is depicted in SEQ ID NO:10. In some embodiments, the VL includes a sequence being at least 75%, at least 80%, at least 85%, at least 90%, at least 95%, at least 97%, or at least 99% identical to an amino acid sequence as depicted in SEQ ID NO: 10.

An exemplary LC having SEQ ID NO:1 SEQ ID NO:3, and SEQ ID NO:4 includes a sequence being at least 75%, at least 80%, at least 85%, at least 90%, at least 95%, at least 97%, or at least 99% identical to an amino acid sequence as depicted in SEQ ID NO:11.

In one embodiment, the antibody of the present disclosure comprises a heavy chain (HC) comprising SEQ ID NO:19, and a light chain (LC) comprising SEQ ID NO:11.

In one embodiment, the antibody of the present disclosure comprises a heavy chain (HC) consisting of SEQ ID NO:19, and a light chain (LC) consisting of SEQ ID NO:11.

In some embodiments, the disclosure provides an antibody having SEQ ID NO:2 and SEQ ID NO:5. In some embodiments, the antibody further includes an LCDR2 as depicted in SEQ ID NO:3.

In some embodiments, the disclosure provides a VL that includes SEQ ID NO:2, SEQ ID NO: 3, and SEQ ID NO:5. An exemplary VL is depicted in SEQ ID NO:12. In some embodiments, the VL includes a sequence being at least 75%, at least 80%, at least 85%, at least 90%, at least 95%, at least 97%, or at least 99% identical to an amino acid sequence as depicted in SEQ ID NO:12.

An exemplary LC having SEQ ID NO:2 SEQ ID NO:3 and SEQ ID NO:5 includes a sequence being at least 75%, at least 80%, at least 85%, at least 90%, at least 95%, at least 97%, or at least 99% identical to an amino acid sequence as depicted in SEQ ID NO:13.

In one embodiment, the antibody of the present disclosure comprises a heavy chain (HC) comprising SEQ ID NO:19, and a light chain (LC) comprising SEQ ID NO:13.

In one embodiment, the antibody of the present disclosure comprises a heavy chain (HC) consisting of SEQ ID NO:19 and a light chain (LC) consisting of SEQ ID NO:13.

In some embodiments, the disclosure provides an antibody having SEQ ID NO:1 and SEQ ID NO:6. In some embodiments, the antibody further includes an LCDR2 as depicted in SEQ ID NO:3.

In some embodiments, the disclosure provides a VL that includes SEQ ID NO:1, SEQ ID NO: 3, and SEQ ID NO:6. An exemplary VL is depicted in SEQ ID NO:14. In some embodiments, the VL includes a sequence being at least 75%, at least 80%, at least 85%, at least 90%, at least 95%, at least 97%, at least 97%, or at least 99% identical to an amino acid sequence as depicted in SEQ ID NO:14.

An exemplary LC having SEQ ID NO:1, SEQ ID NO:3, and SEQ ID NO:5 includes a sequence being at least 75%, at least 80%, at least 85%, at least 90%, at least 95%, at least 97%, or at least 99% identical to an amino acid sequence as depicted in SEQ ID NO:15.

In some embodiments, an antibody of the disclosure includes a HCDR1 including SEQ ID NO:7, a HCDR2 including SEQ ID NO:8, a HCDR3 including SEQ ID NO:9, a LCDR1 including SEQ ID NO:1 or SEQ ID NO:2, a LCDR2 including SEQ ID NO:3, and a LCDR3 including SEQ ID NO:4, SEQ ID NO:5, or SEQ ID NO:6.

In one embodiment, the antibody of the present disclosure comprises a heavy chain (HC) comprising SEQ ID NO:19, and a light chain (LC) comprising SEQ ID NO:15.

In one embodiment, the antibody of the present disclosure comprises a heavy chain (HC) consisting of SEQ ID NO:19 and a light chain (LC) consisting of SEQ ID NO:15.

Exemplary CDRs of Antibodies of the Present Disclosure

TABLE 1
Exemplary CDRs of Antibodies of the Present Disclosure

| Antibody | HCDR1 | HCDR2 | HCDR3 | LCDR1 | LCDR2 | LCDR3 |
|---|---|---|---|---|---|---|
| Antibody | (SEQ ID | (SEQ ID | (SEQ ID | (SEQ ID | (SEQ ID | (SEQ ID |
| A | NO: 7) | NO: 8) | NO: 9) | NO: 1) | NO: 3) | NO: 4) |
| Antibody | (SEQ ID | (SEQ ID | (SEQ ID | (SEQ ID | (SEQ ID | (SEQ ID |
| B | NO: 7) | NO: 8) | NO: 9) | NO: 2) | NO: 3) | NO: 5) |
| Antibody | (SEQ ID | (SEQ ID | (SEQ ID | (SEQ ID | (SEQ ID | (SEQ ID |
| C | NO: 7) | NO: 8) | NO: 9) | NO: 1) | NO: 3) | NO: 6) |
| Antibody | (SEQ ID | (SEQ ID | (SEQ ID | (SEQ ID | (SEQ ID | (SEQ ID |
| D | NO: 7) | NO: 8) | NO: 9) | NO: 1) | NO: 3) | NO: 5) |

In certain embodiments, the HCDR1, HCDR2, HCDR3, LCDR1, LCDR2, and LCDR3 amino acid sequences set is selected from SEQ ID NOs: 1, 2, 3, 4, 5, 6, 7, 8, and 9.

Exemplary antibodies of the present disclosure include an antibody having LC as depicted by SEQ ID NO:11 and HC as depicted by SEQ ID NO:19. Another exemplary of the present disclosure includes an antibody having LC as depicted by SEQ ID NO: 13 and HC as depicted by SEQ ID NO:19. Another exemplary of the present disclosure includes an antibody having LC as depicted by SEQ ID NO:15 and HC as depicted by SEQ ID NO:19. Another exemplary of the present disclosure includes an antibody having LC as depicted by SEQ ID NO:17 and HC as depicted by SEQ ID NO:19.

In some embodiments, the LC and or HC include a signal peptide. An exemplary signal peptide includes SEQ ID NO:20 and the corresponding cDNA is depicted in SEQ ID NO: 21.

Percent identity, as used in the present disclosure, in the context of two or more amino acid sequence refers to two or more sequences having a specified percentage of amino acid residues that are the same, when compared and aligned for maximum correspondence, as measured using a sequence comparison algorithm (e.g., BLASTP and BLASTN or other algorithms available to persons of skill) or by visual inspection. Depending on the application, the percent identity can exist over a region of the sequence being compared, e.g., over a functional domain, or, alternatively, exist over the full length of the two sequences to be compared. By way of example, percent identity of a sequence may be compared to a reference sequence. For example, when using a sequence comparison algorithm, test and reference sequences may be input into a computer (and subsequence coordinates may be further designated if desired along with sequence algorithm program parameters). The sequence comparison algorithm then calculates the percent sequence identity for the test sequence(s) relative to the reference sequence(s), based on the designated program parameters. Exemplary sequence alignment and/or homology/identity algorithms are available through, Smith & Waterman, Adv. Appl. Math. 2:482 (1981), Needleman & Wunsch, J. Mol. Biol. 48:443 (1970), Pearson & Lipman, Proc. Nat'l. Acad. Sci. USA 85:2444 (1988), GAP, BESTFIT, FASTA, and TFASTA (in the Wisconsin Genetics Software Package, Genetics Computer Group, 575 Science Dr., Madison, Wis.), or by visual inspection (see generally Ausubel et al., infra). One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The present disclosure provides antibodies that bind to human LEPR and agonize LEPR. Certain antibodies of the present disclosure that bind to and agonize LEPR have one or more of the following properties: bind LEPR of several species with desirable binding affinities, activate LEPR of several species, beneficial properties with regard to binding of Fcγ receptors, do not desensitize LEPR signaling, activate signaling deficient LEPR, and can be advantageously combined with a secondary therapeutic agent.

The term “agonize”, as used herein, refers to the ability of an antibody, antibody fragment, or binding molecule to induce or increase one or more activities or functions associated with an antigen. An antigen is the molecule to which the antibody, antibody fragment, or binding molecule binds. The antigen may be a receptor (eg, LEPR).

As used herein, agonism and percent agonism is determined by methods known in the art and/or by methods essentially as described herein, including, for example, in Example 4 below.

The term “bind” and “binds” as used herein, are intended to mean, unless indicated otherwise, the ability of a protein or molecule to form a chemical bond or attractive interaction with another protein or molecule, which results in proximity of the two proteins or molecules as determined by common methods known in the art.

The terms “selectively binds” or “specifically binds” mean that an antibody of the disclosure interacts more frequently, more rapidly, with greater duration, with greater affinity, or with some combination of the above to human and/or cynomolgus and/or canine LEPR than to other substances. An antibody may be said to bind specifically to an antigen if it binds at least 25% greater, at least 50% greater, at least 100% greater, at least 200% greater, or at least 500% greater than it binds a different antigen or non-antigen target as measured by a technique known in the art. Exemplary techniques include competition ELISA or K_{D} measurements via SPR at 25° C. or 30° C.

In some embodiments, an antibody of the present disclosure binds to human LEPR, with a K_{D} of 5.0E-7 M or less. In another embodiment, an antibody of the present disclosure binds to human LEPR, with a K_{D} of 5.0E-8 M or less. In another embodiment, an antibody of the present disclosure binds to human LEPR, with a K_{D} of 2.0E-8 M or less.

In some embodiments, an antibody of the present disclosure binds to human LEPR, with a K_{D} of between 1E-10 M and 5.0E-7 M. In some embodiments, an antibody of the present disclosure binds to human LEPR, with a K_{D} of between 1E-10 M and 5.0E-8 M. In some embodiments, an antibody of the present disclosure binds to human LEPR, with a K_{D} of between 1E-10 M and 3.0E-8 M. In some embodiments, an antibody of the present disclosure binds to human LEPR, with a K_{D} of between 1E-10 M and 2.0E-8 M. In some embodiments, an antibody of the present disclosure binds to human LEPR, with a K_{D} of between 1E-9 M and 2.5E-8 M. In some embodiments, an antibody of the present disclosure binds to human LEPR, with a K_{D} of between 1E-9 M and 2.0E-8 M.

In some embodiments, an antibody of the present disclosure binds to cynomolgus LEPR, with a K_{D} of 5.0E-7 M or less. In another embodiment, an antibody of the present disclosure bind to cynomolgus LEPR, with a K_{D} of 5.0E-8 M or less. In another embodiment, an antibody of the present disclosure binds to cynomolgus LEPR, with a K_{D} of 2.0E-8 M or less.

In some embodiments, an antibody of the present disclosure binds to cynomolgus LEPR, with a K_{D} of between 1E-10 and 5.0E-7 M. In some embodiments, an antibody of the present disclosure binds to cynomolgus LEPR, with a K_{D} of between 1E-10 M and 5.0E-8 M. In some embodiments, an antibody of the present disclosure binds to cynomolgus LEPR, with a K_{D} of between 1E-10 and 2.0E-8 M. In some embodiments, an antibody of the present disclosure binds to cynomolgus LEPR, with a K_{D} of between 1E-9 and 2.0E-8 M.

In some embodiment, an antibody of the present disclosure binds to canine LEPR, with a K_{D} of 5.0E-6 M or less. In another embodiment, an antibody of the present disclosure binds to canine LEPR, with a K_{D} of 5.0E-7 M or less. In another embodiment, an antibody of the present disclosure binds to canine LEPR, with a K_{D} of 2.0E-7 M or less. In another embodiment, an antibody of the present disclosure binds to canine LEPR, with a K_{D} of 1.5E-7 M or less.

In some embodiments, an antibody of the present disclosure binds to canine LEPR, with a K_{D} of between 1E-10M and 5.0E-6 M. In some embodiments, an antibody of the present disclosure binds to canine LEPR, with a K_{D} of between 1E-10M and 5.0E-7 M. In some embodiments, an antibody of the present disclosure binds to canine LEPR, with a K_{D} of between 1E-10M and 3.5E-7 M. In some embodiments, an antibody of the present disclosure binds to canine LEPR, with a K_{D} of between 1E-10 M and 1.5E-7 M. In some embodiments, an antibody of the present disclosure binds to canine LEPR, with a K_{D} of between 1E-8 M and 1.5E-7 M.

In some embodiments, an antibody of the present disclosure provides K_{D} (human) of between 1E-9 M and 2.0E-8 M, a K_{D} (cynomolgus) of between 1E-9 and 2.0E-8 M, and a K_{D} (canine) of between 1E-9 M and 1.5E-7 M.

As used herein, K_{D} is determined by methods known in the art and/or by methods essentially as described herein, including, surface plasmon resonance (SPR) at 25° C. or 30° C. An exemplary method is provided in Example 3, below.

In some embodiments, an antibody of the present disclosure has an EC₅₀ (humans) of 0.50 nM or less. In another embodiment, an antibody of the present disclosure has an EC₅₀ (humans) of 0.40 nM or less. In another embodiment, an antibody of the present disclosure has an EC₅₀ (humans) of 0.30 nM or less. In another embodiment, the antibodies of the present disclosure have an EC₅₀ (humans) of 0.25 nM or less. In another embodiment, an antibody of the present disclosure have an EC₅₀ (humans) of 0.20 nM or less.

In some embodiments, an antibody of the present disclosure has an EC₅₀ (humans) of between 0.50 nM and 0.01 nM. In some embodiments, an antibody of the present disclosure has an EC₅₀ (humans) of between 0.40 nM and 0.01 nM. In some embodiments, an antibody of the present disclosure has an EC₅₀ (humans) of between 0.30 nM and 0.01 nM. In some embodiments, an antibody of the present disclosure has an EC₅₀ (humans) of between 0.25 nM and 0.01 nM. In some embodiments, an antibody of the present disclosure has an EC₅₀ (humans) of between 0.20 nM and 0.01 nM.

In some embodiments, an antibody of the present disclosure has an EC₅₀ (cynomolgus) of 2 nM or less. In another embodiment, an antibody of the present disclosure has an EC₅₀ (cynomolgus) of 1.5 nM or less. In another embodiment, an antibody of the present disclosure has an EC₅₀ (cynomolgus) of 1.0 nM or less. In another embodiment, an antibody of the present disclosure has an EC₅₀ (cynomolgus) of 0.9 nM or less. In another embodiment, an antibody of the present disclosure has an EC₅₀ (cynomolgus) of 0.8 nM or less.

In some embodiments, an antibody of the present disclosure has an EC₅₀ (cynomolgus) of between 2.0 nM and 0.01 nM. In some embodiments, an antibody of the present disclosure has an EC₅₀ (cynomolgus) of between 1.5 nM and 0.01 nM. In some embodiments, an antibody of the present disclosure has an EC₅₀ (cynomolgus) of between 1.0 nM and 0.01 nM. In some embodiments, an antibody of the present disclosure has an EC₅₀ (cynomolgus) of between 0.90 nM and 0.01 nM.

In some embodiments, an antibody of the present disclosure has an EC₅₀ (canine) of 5 nM or less. In another embodiment, an antibody of the present disclosure has an EC₅₀ (canine) of 2.5 nM or less. In another embodiment, an antibody of the present disclosure has an EC₅₀ (canine) of 2.0 nM or less. In another embodiment, an antibody of the present disclosure has an EC₅₀ (canine) of 1.5 nM or less. In another embodiment, an antibody of the present disclosure has an EC₅₀ (canine) of 1.0 nM or less. In another embodiment, an antibody of the present disclosure has an EC₅₀ (canine) of 0.9 nM or less. In another embodiment, an antibody of the present disclosure has an EC₅₀ (canine) of 0.8 nM or less. In another embodiment, an antibody of the present disclosure has an EC₅₀ (canine) of 0.7 nM or less.

In some embodiments, an antibody of the present disclosure has an EC₅₀ (canine) of between 5.0 nM and 0.01 nM. In some embodiments, an antibody of the present disclosure has an EC₅₀ (canine) of between 2.5 nM and 0.01 nM. In some embodiments, an antibody of the present disclosure has an EC₅₀ (canine) of between 2.0 nM and 0.01 nM. In some embodiments, an antibody of the present disclosure has an EC₅₀ (canine) of between 0.90 nM and 0.01 nM. In some embodiments, an antibody of the present disclosure has an EC₅₀ (canine) of between 0.80 nM and 0.01 nM. In some embodiments, an antibody of the present disclosure has an EC₅₀ (canine) of between 0.70 nM and 0.01 nM.

In one embodiment, an antibody of the present disclosure provides EC₅₀ (humans) of between 0.25 nM and 0.01 nM, an EC₅₀ (cynomolgus) of between 1.0 nM and 0.01 nM, and an EC₅₀ (canine) of between 0.70 nM and 0.01 nM.

As used herein, EC₅₀ is determined by methods known in the art and/or by methods essentially as described herein, including, for example, by phosphorylation of intracellular STAT3 assay. An exemplary method is provided in Example 4, below.

In one embodiment, an antibody of the present disclosure provides at least 45%, at least 50%, at least 55%, or at least 60% agonism of human LEPR, as compared to human leptin. In one embodiment, an antibody of the present disclosure provides between 45% and 75% agonism of human LEPR, as compared to human leptin. In one embodiment, an antibody of the present disclosure provides between 50% and 70% agonism of human LEPR, as compared to human leptin. In one embodiment, an antibody of the present disclosure provides between 55% and 65% agonism of human LEPR, as compared to human leptin.

In one embodiment, an antibody of the present disclosure provides at least 20%, at least 25%, at least 30%, or at least 35% agonism of cynomolgus LEPR, as compared to human leptin. In one embodiment, an antibody of the present disclosure provides between 20% and 50% agonism of cynomolgus LEPR, as compared to human leptin. In one embodiment, an antibody of the present disclosure provides between 20% and 40% agonism of cynomolgus LEPR, as compared to human leptin. In one embodiment, an antibody of the present disclosure provides between 20% and 30% agonism of cynomolgus LEPR, as compared to human leptin.

In one embodiment, an antibody of the present disclosure provides at least 45%, at least 50%, at least 55%, at least 60%, at least 70%, at least 75%, at least 80%, or at least 85% agonism of canine LEPR, as compared to human leptin.

In one embodiment, an antibody of the present disclosure provides between 45% and 85% agonism of canine LEPR, as compared to human leptin. In one embodiment, an antibody of the present disclosure provides between 50% and 85% agonism of canine LEPR, as compared to human leptin. In one embodiment, an antibody of the present disclosure provides between 65% and 85% agonism of canine LEPR, as compared to human leptin. In one embodiment, an antibody of the present disclosure provides between 65% and 80% agonism of canine LEPR, as compared to human leptin.

In one embodiment, an antibody of the present disclosure provides agonism of human LEPR at 50-60%, agonism of cynomolgus LEPR at greater than or equal to 20%, and agonism of canine LEPR at 70-80%; as compared to human leptin.

The present disclosure provides antibodies that selectively bind to human, cynomolgus (Macaca fascicularis), and canine (Canis lupus dingo) LEPR. In some an embodiments, the present disclosure provides an antibody that selectively bind to human and canine LEPR.

The present disclosure provides antibodies that do not de-sensitize LEPR signaling.

The present disclosure provides nucleic acids encoding a heavy chain or light chain of antibodies that bind and agonize the leptin receptor (LEPR) or vectors comprising such nucleic acids.

Exemplary nucleic acids include a nucleic acid sequence that encodes for a polypeptide sequence depicted in any of SEQ ID NO:11, 13, 15, 17, and 19. Exemplary nucleic acids include SEQ ID NO:22, 23, 24, 25, and 26.

The terms “nucleic acid” or “polynucleotide”, as used interchangeably herein, refer to polymers of nucleotides, including single-stranded and/or double-stranded nucleotide-containing molecules, such as DNA, cDNA, and RNA molecules, incorporating native, modified, and/or analogs of, nucleotides. Polynucleotides of the present disclosure may also include substates incorporated therein, for example, by DNA or RNA polymerase or a synthetic reaction.

Polynucleotides of the present disclosure may be expressed in a host cell, for example after the polynucleotides have been operably linked to an expression control sequence. Expression control sequences capable of expression of polynucleotides to which they are operably linked are well known in the art. For example, an expression vector may include a sequence that encodes one or more signal peptides that facilitate secretion of the polypeptide(s) from a host cell. The signal peptide may be an immunoglobulin signal peptide or a heterologous signal peptide, for example. Expression vectors containing a polynucleotide of interest (e.g., a polynucleotide encoding a polypeptide of an antibody) may be transferred into a host cell by well-known methods. Additionally, expression vectors may contain one or more selection markers, e.g., tetracycline, neomycin, and dihydrofolate reductase, to aide in detection of host cells transformed with desired polynucleotide sequences.

Some embodiments of the present disclosure provide vectors comprising a nucleic acid sequence encoding an antibody heavy chain or light chain, wherein said antibody specifically bind to and agonize the leptin receptor. In specific embodiments, the vector includes a nucleic acid according to at least one of SEQ ID NOs: 11, 13, 15, 17, 19, 22, 23, 24, 25, and 26.

In another aspect, the present disclosure provides a host cell comprising one or more of SEQ ID NO:11, 13, 15, 17, 19, 22, 23, 24, 25, and 26.

In some embodiments, the cell, e.g., host cell, includes a vector having a first nucleic acid sequence encoding at least one of SEQ ID NOs: 11, 13, 15, 17, 19, 22, 23, 24, 25, and 26., and a second nucleic acid sequence encoding at least one of SEQ ID NOs: 11, 13, 15, 17, 19, 22, 23, 24, 25, and 26.

A host cell includes cells stably or transiently transfected, transformed, transduced, or infected with one or more expression vectors expressing all or a portion of an antibody of the present disclosure. According to some embodiments, a host cell may be stably or transiently transfected, transformed, transduced, or infected with an expression vector expressing HC polypeptides and an expression vector expressing LC polypeptides of an antibody of the present disclosure. In some embodiments, a host cell may be stably or transiently transfected, transformed, transduced, or infected with an expression vector expressing HC and LC polypeptides of an antibody of the present disclosure. The antibody of the present disclosure may be produced in mammalian cells such as CHO, NSO, HEK293, or COS cells according to techniques well known in the art.

The present disclosure further provides a process for producing an antibody or antigen binding fragments thereof that specifically binds leptin receptor described herein by culturing the host cell described above, e.g., a mammalian host cell, under conditions such that the antibody is expressed and recovering the expressed antibody from the culture medium.

Medium, into which an antibody of the present disclosure has been secreted, may be purified by conventional techniques, such as mixed-mode methods of ion-exchange and hydrophobic interaction chromatography. For example, the medium, may be applied to and eluted from Protein A or G column using conventional methods; mixed-mode methods of ion-exchange and hydrophobic interaction chromatography may also be used. Soluble aggregate and multimers may be effectively removed by common techniques, including size exclusion, hydrophobic interaction, ion exchange, or hydroxyapatite chromatography. The product may be immediately frozen, for example at −70°° C., refrigerated, or may be lyophilized. Various methods of protein purification may be employed, and such methods are known in the art and described, for example, in Deutscher, Methods in Enzymology 182:83-89 (1990) and Scopes, Protein Purification: Principles and Practice, 3^{rd} Edition, Springer NY (1994).

The present disclosure further provides antibodies or antigen binding fragments thereof produced by any of the processes described herein.

Mammalian expression of antibodies typically results in glycosylation. Glycosylation of antibodies is typically either N-linked or O-linked. N-linked glycosylation refers to the attachment of a carbohydrate moiety to the side chain of an asparagine residue. O-linked glycosylation refers to the attachment of a sugar, for example N-acetylgalactosamine, galactose, or xylose to a serine or threonine. Typically, glycosylation occurs in the Fc region of the antibody at a highly conserved N-glycosylation site (e.g., position 297 in IgG1, according to IMGT or EU Index numbering). Glycosylation sites can be modified to alter glycosylation (e.g., blocking or reducing glycosylation or altering the amino acid sequence to produce additional or diverse glycosylation).

Mammalian expression of antibodies from IgG subclasses can result in processing of C-terminal amino acids from one or both heavy chains; for example, one or two C-terminal amino acids can be removed for IgG1 antibodies. For example, IgG1 antibodies under certain circumstances, if a C-terminal lysine is present, then it may be truncated or clipped off from the heavy chain during expression. Additionally, a penultimate glycine may also be truncated or clipped off from the heavy chain as well.

Mammalian expression of antibodies can also result in the modification of N-terminal amino acids. For example, where the N-terminal most amino acid of a heavy chain or light chain is a glutamine, it may be modified into pyro-glutamic acid.

In some embodiments, the disclosure provides a pharmaceutical composition including antibody disclosed herein and above. The pharmaceutical composition may further include at least one of a pharmaceutically acceptable excipient, diluent, or carrier.

In some embodiments, the disclosure provides a pharmaceutical composition including an antibody that binds and agonizes LEPR (disclosed herein) and a second therapeutic agent.

In some embodiments, the second therapeutic agent restores leptin sensitivity.

In some embodiments, the second therapeutic agent includes an agent that has amylin receptor agonist activity. Exemplary agents having amylin receptor agonist activity include amylin or amylin analogs. Examples of agents having amylin receptor agonist activity include petrelintide, cagrilintide, and pramlintide.

In another embodiment, the second therapeutic agent has amylin receptor agonist activity and calcitonin receptor agonist activity.

In some embodiments, the second therapeutic agent includes an agent selected from SEQ ID NO:39-47.

In some embodiments, the second therapeutic agent includes an agent selected from SEQ ID NO:48-49.

In some embodiments, the second therapeutic agent includes an agent selected from SEQ ID NO:50-51.

In some embodiments, the second therapeutic agent has GLP-1 (Glucagon-like peptide-1) receptor agonist activity. In some embodiments, the second therapeutic agent having GLP-1 receptor agonist activity is an antibody, small molecule, protein, polypeptide, peptide, or aptamer. In some embodiments, the second therapeutic having GLP-1 receptor agonist activity is selected from exenatide, exenatide extended-release, dulaglutide, liraglutide, lixisenatide, semaglutide, cotadutide, noiiglutide, oxyntomodulin, mazdutide, retatrutide, tirzepatide, albiglutide, beinaglutide PEG-loxenatide, pemvidutide, albiglutide, extendin-4, survodutide, pemvidutide, taspoglutide, efpeglenatide, lotiglipron, danuglipron, vurolenatide, dapiglutide, cinchonine, efocipegtrutide, efinopegdutide, ecnoglutide, orforglipron, lotiglipron, and danuglipron.

In some embodiments, the second therapeutic agent includes a compound of Formula:

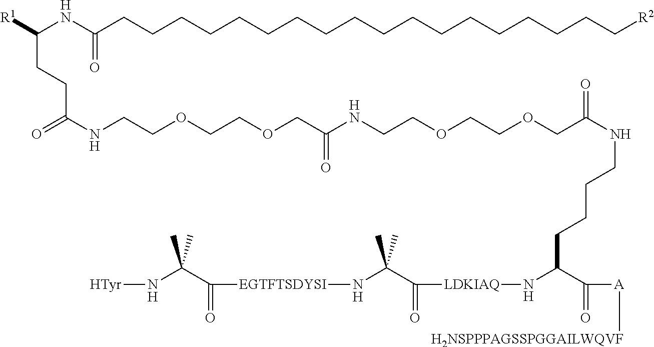

Or a pharmaceutically acceptable salt thereof, wherein: R1 is selected from the group consisting of —C(═O)(OZ1), —P(═O)(X)(Y) and a 5-10 membered heteroaryl containing 1-2 heteroatoms selected from N, O and S optionally substituted with 1-2 R7 independently selected from halogen, C1-6 alkyl, haloC1-6 alkyl, haloC1-6 alkoxy,-OR5, C3-10 cycloalkyl, C6-10 aryl, 5-10 membered heteroaryl, and 5-10 membered heterocyclyl; R2 is selected from the group consisting of —C(═O)(OZ2), —P(═O)(X)(Y) and a 5-10 membered heteroaryl containing 1-2 heteroatoms selected from N, O and S optionally substituted with 1-2 R7 independently selected from halogen, C1-6 alkyl, haloC1-6 alkyl, haloC1-6 alkoxy, —OR5, C3-10 cycloalkyl, C6-10 aryl, 5-10 membered heteroaryl, and 5-10 membered heterocyclyl; each R7 is independently selected from the group consisting of halogen, C1-6 alkyl, haloC1-6 alkyl, haloC1-6 alkoxy, C1-6 alkoxy, C3-10 cycloalkyl, C6-10 aryl, 5-10 membered heteroaryl and 5-10 membered heterocyclyl; X and Y each are independently selected from the group consisting of-OR4, NR5R6, C1-6 alkyl and haloC1-6 alkyl; each R4 is independently selected from the group consisting of hydrogen, C1-6 alkyl, haloC1-6 alkyl, C6-10 aryloxy, and C6-10 aryl alkoxy; each R5 is independently hydrogen or C1-6 alkyl; each R6 is independently hydrogen or C1-6 alkyl; and Z1 and Z2 each are independently selected from the group consisting of hydrogen, C1-6 alkyl, haloC1-6 alkyl, haloC1-6 alkoxy, C1-6 alkoxy, C3-10 cycloalkyl and C6-10 aryl. In some embodiments, at least one of Z1 and Z2 is not hydrogen.

In some embodiments, the second therapeutic agent includes a compound of Formula:

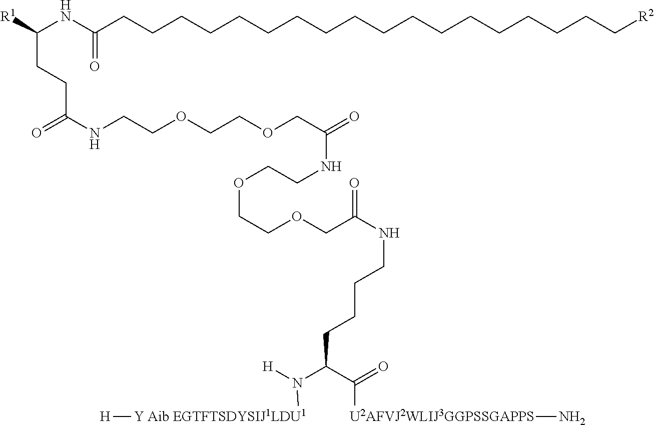

or a pharmaceutically acceptable salt thereof, wherein:

Aib is 2-aniinoisobutync acid; each instance of J1, J2, and J3 is independently an ammo acid selected from Aib, a naturally occurring ammo acid, and an unnatural amino acid;
U1 is -(J4)n1-(J5)n2-(J6)n3-(J7)n4-;
U2 is -(J8)n5-(J9)n6-(J10)n7-(J11)n8-;
each instance of J4, J5, J6, J7, J8, J9, J10, and J11 is independently a naturally occurring ammo acid or an unnatural amino acid; each of n1, n2, n3, n4, n5, n6, n7,and n8 is independently 0 or 1, provided that the sum n1+n2+n3+n4+n5+n6+n7+n8 is 4;

R1 is selected from the group consisting of —C(═O) (OZ1), —P(═O)(X)(Y) and a 5-10 membered heteroaryl containing 1-2 heteroatoms selected from N, O, and S, the heteroaryl optionally substituted with 1-2 R7 independently selected from halogen, C1-6 alkyl, haloC1-6 alkyl, haloC1-6 alkoxy, OR5, C3-10 cycloalkyl, C6-10 aryl, 5-10 membered heteroaryl and 5-10 membered heterocyclyl; R2 is selected from the group consisting of —C(═O)(OZ2), —P(═O)(X)(Y) and a 5-10 membered heteroaryl containing 1-2 heteroatoms selected from N, O, and S, the heteroaryl optionally substituted with 1-2 R7 independently selected from halogen, C1-6 alkyl, haloC1-6 alkyl, haloC1-6 alkoxy, —OR3, C3-10 cycloalkyl, C6-10 aryl, 5-10 membered heteroaryl and 5-10 membered heterocyclyl; each Rz is independently selected from the group consisting of halogen, C1-6 alkyl, haloC1-6 alkyl, haloC1-6 alkoxy, C1-6 alkoxy, C3-10 cycloalkyl, C6-10 aryl, 5-10 membered heteroaryl, and 5-10 membered heterocyclyl;

X and Y each are independently selected from the group consisting of-OR4, NR5R6, C1-6 alkyl and haloC1-6 alkyl; each R4 is independently selected from the group consisting of hydrogen C1-6 alkyl, haloC1-6 alkyl, C6-10 aryl, and C7-11 arylalkyl; each R5 is independently hydrogen or C1-6 alkyl; each R6 is independently hydrogen or C1-6 alkyl; and

Z1 and Z2 each are independently selected from the group consisting of hydrogen, C1-6 alkyl, haloC1-6 alkyl, haloC1-6 alkoxy, C1-6 alkoxy, C3-10 cycloalkyl, and C6-10 aryl.

In some embodiments, the second therapeutic agent includes a compound of Formula:

YX1EGTFTSDYSIX2LDKIAQKAX3VQWLIAGGPSSGAPPPS;

wherein

X1 is Aib;

X2 is Aib;

K at position 20 is chemically modified through conjugation to the epsilon-amino group of the K side-chain with ([2-(2-Amino-ethoxy)-ethoxy]-acetyl) 2-(γGlu)a-CO-(CH2)b-CO2H wherein a is 1 to 2 and b is 10 to 20;

X3 is Phe or 1-Nal;

and the C-terminal amino acid is optionally amidated as a C-terminal primary amide.

“Aib” is alpha amino isobutyric acid, and “1-Nal” is 1-Naphthylalanine.

In some embodiments, the second therapeutic agent having GLP-1 receptor agonist activity is selected from SEQ ID NO:52-56.

In some embodiments, the second therapeutic agent is an agent that exhibits one or more of: amylin receptor agonist activity, has GLP-1 receptor agonist activity, and glucagon receptor agonist activity.

In some embodiments, the second therapeutic agent having GLP-1 receptor agonist activity is a dual GLP-1 receptor agonist and GIP receptor agonist. In some embodiments, the second therapeutic agent having GLP-1 receptor agonist activity is a dual GLP-1 receptor agonist and GCG receptor agonist. In some embodiments, the second therapeutic agent includes a dual agonist of the GLP-1 and GIP receptors (GLP1R/GIPR). An example of a dual agonist of the GLP-1 and GIP receptors includes a compound depicted in SEQ ID NO: 53 or SEQ ID NO:54.

In some embodiments, the second therapeutic agent having GLP-1 receptor agonist activity is a triagonist of GLP-1, GIP (glucose-dependent insulinotropic polypeptide), and glucagon receptors (GIPR/GLP1R/GCGR). An example of a second therapeutic agent that is a triagonist of GIP/GLP-1/glucagon receptors includes a compound depicted in SEQ ID NO: 55 or SEQ ID NO:56.

In some embodiments, the second therapeutic agent has glucagon receptor agonist activity. Exemplary agents include glucagon or glucagon analog. An example of an agent having glucagon receptor agonist activity includes SEQ ID NO:57.

In another aspect, the present disclosure provides a method of treating a disease or disorder associated with or mediated by leptin deficiency, leptin resistance, or otherwise treatable by agonizing LEPR. The method includes administering a therapeutically effective amount of an antibody or composition of the present disclosure to a subject in need thereof.

The term “treatment” or “treating” as used herein, refers to all processes wherein there may be a slowing, controlling, delaying, or stopping of the progression of the disorders or diseases disclosed herein, or ameliorating disorder or disease symptoms, but does not necessarily indicate a total elimination of all disorder or disease symptoms. Treatment includes administration of a protein or nucleic acid or vector or composition for treatment of a disease or disorder in a patient, particularly in a human.

The term “therapeutically effective amount” as used herein, refers to an amount of a compound or composition necessary to achieve a desired result.

In an embodiment, the present disclosure provides a method of treating disorders or diseases associated with signaling deficient LEPR by administering an antibody or composition of the present disclosure, to a subject in need thereof. An example of a signaling deficient LEPR include LEPR having the A409E mutation.

In some embodiments, the present disclosure provides a method of treating a disease or disorder associated with or mediated by leptin deficiency by administering an antibody or composition of the present disclosure to a subject in need thereof, wherein the disease or disorder includes a lipodystrophy disorder. Examples of lipodystrophy disorders include congenital generalized lipodystrophy, acquired generalized lipodystrophy, familial partial lipodystrophy, acquired partial lipodystrophy, centrifugal abdominal lipodystrophy, lipoatrophia annularis, and localized lipodystrophy.

In an embodiment, the present disclosure provides a method of inducing weight loss, weight management, chronic weight management, or treating obesity by administering an antibody or composition of the present disclosure to a subject. Wherein the method includes administering an antibody or composition of the present disclosure to a subject in need thereof.

The term “obesity” as used herein, refers to a disease or disorder involving excess body fat that increases the risk of health problems. In an embodiment, the term “obesity” includes an adult subject having a body mass index (BMI) of 30 kg/m2 or greater. In another embodiment, the term “obesity” includes an adult subject having a BMI of 25 kg/m2 or greater. “Chronic weight management” refers to a desired reduction in body weight or maintenance of body weight.

In an embodiment, the present disclosure provides a method for chronic weight management in a subject in need of additional weight management; wherein the subject has obesity or is overweight. Wherein the method of chronic weight management includes administering an antibody or composition of the present disclosure to a subject in need thereof.

In an embodiment, the term “overweight” includes an adult subject having a BMI of 27 kg/m2 or greater. In another embodiment, the term “overweight” includes an adult subject having a BMI of 23 kg/m2 or greater. In another embodiment “overweight” includes an adult subject having a BMI of 23-30, 23-27, 25-30, 27-30, 23-25, or 25-27 kg/m2.

In one embodiment, “chronic weight management” means a method by which one maintains weight loss as an adjunct to reduced calorie diet and increased physical activity in persons who are currently or have been previously characterized as having obesity or is overweight. Wherein the method of chronic weight management includes administering an antibody or composition of the present disclosure to a subject in need thereof.

In an embodiment, the present disclosure provides a method of inducing weight loss in a subject having a BMI of 23-30, 23-27, 25-30, 27-30, 23-25, or 25-27 kg/m2, wherein the method includes administering an antibody or composition of the present disclosure to the subject.

The antibodies or compositions of the present disclosure may be administered as a monotherapy or as a combination therapy. In an embodiment, combination therapy includes simultaneous, separate, or sequential combination of an antibody disclosed herein and a second therapeutic agent to induce weight loss, weight management, chronic weight management, or treat obesity. In an embodiment, combination therapy includes simultaneous, separate, or sequential combination of an antibody disclosed herein and a second therapeutic agent to treat a disease or disorder associated with or mediated by leptin deficiency, leptin resistance, or otherwise treatable by agonizing LEPR.

Second therapeutic agents to be administered in combination with an LEPR agonist antibody are disclosed herein. In some embodiments, the second therapeutic agent includes a therapeutic agent that restores leptin sensitivity.

In an embodiment, the disclosure provides an antibody or composition of the present disclosure for use in therapy, chronic weight management, or treatment of obesity.

In an embodiment, the disclosure provides for the use of antibody of an antibody disclosed herein, in the manufacture of a medicament for therapy, chronic weight management, or treatment of obesity.

In some embodiments, the second therapeutic agent is an agent that exhibits one or more of: amylin receptor agonist activity, GLP-1 receptor agonist activity, GIP receptor agonist activity, and glucagon receptor agonist activity. Exemplary second therapeutic agents are depicted in SEQ ID NO: 39-57.

As used herein, the words “a”, “an”, and “the” denote “one or more”.

As used herein, antibodies, small molecules, proteins, peptides, or aptamers of the present disclosure include the respective free base forms and pharmaceutically acceptable salts thereof.

### EXAMPLES

### Example 1

### Antibody Expression and Purification

Antibodies A, B, C, and D were each generated in a mammalian cell expression system using CHOK1 cell derivatives (Lonza Biologics Inc.). The cDNA sequences encoding the antibodies were subcloned into GS-containing expression plasmid backbones (pEE12.4 based plasmids; Lonza Biologics Inc.).

TABLE 2
Exemplary Antibodies

| | Amino Acid | cDNA |
|---|---|---|
| Antibody A | | |
| HC | SEQ ID NO: 19 | SEQ ID NO: 26 |
| LC | SEQ ID NO: 11 | SEQ ID NO: 22 |
| Antibody B | | |
| HC | SEQ ID NO: 19 | SEQ ID NO: 26 |
| LC | SEQ ID NO: 13 | SEQ ID NO: 23 |
| Antibody C | | |
| HC | SEQ ID NO: 19 | SEQ ID NO: 26 |
| LC | SEQ ID NO: 15 | SEQ ID NO: 24 |
| Antibody D | | |
| HC | SEQ ID NO: 19 | SEQ ID NO: 26 |
| LC | SEQ ID NO: 17 | SEQ ID NO: 25 |

The cDNA sequence was fused in-frame with the coding sequence of a signal peptide sequence METDTLLLWVLLLWVPGSTG (SEQ ID NO:20) to enhance secretion of the antibody into the tissue cell medium. The expression was driven by the viral CMV promoter.

For generating the antibody via transient transfection, CHOK1-derived cells were cultured in a bioreactor up to densities between 35-50 e6 vc/mL. The cells were transfected with an equal stoichiometric ratio of the recombinant expression plasmids using a PEI-based method. Briefly, the appropriate volume of CHOK1-derived suspension cells at a density of 20 e6 cells/mL was transferred in shake flasks, and both PEI and recombinant plasmid DNA were added to the cells. Cells were incubated in a suspension culture at 32° C. for 7 days. At the end of the production period, cells were removed by centrifugation and the antibody was purified from the clarified medium.

Alternatively, and for generating the antibody via stable transfections, CHOK1-derived cells were stably transfected using lipid-based transfection reagent and the appropriate amount of recombinant expression plasmid, and the transfected cells were maintained in suspension culture at an adequate cell density. Selection of the transfected cells was accomplished by growth in 25 μM MSX-containing chemically defined serum-free medium and incubated at 32-37° C. and 5-7% CO₂. Subsequently, cells were removed by centrifugation and the antibody was purified from the clarified medium.

The antibody was secreted into the media from the CHO cells, which was subsequently purified by Protein A affinity chromatography followed by cation exchange chromatography. Specifically, the antibody from harvested media was captured onto MabSelect PrismA Protein A resin (Cytiva). The resin was then briefly washed with a wash buffer, such as phosphate-buffered saline (PBS, pH 7.4) or a buffer containing Tris, to remove non-specifically bound material. The protein was eluted from the resin with a low pH solution, such as 10 mM citric acid pH 3. Fractions containing the antibody were pooled. The pH may then be adjusted to approximately 5 by adding a base such as 3 M sodium acetate pH 5.0. The antibody may be further purified by ion exchange chromatography using resins such as POROS 50 HS (ThermoFisher). The antibody may be eluted from the column using a 0 to 1M NaCl gradient in 20 mM sodium acetate, pH 5.0 over 20 column volumes.

The purified antibody may then be concentrated and/or buffer exchanged into phosphate buffered saline or other appropriate formulation buffer through ultrafiltration/diafiltration methods.

### Example 2

### Binding of Antibodies A, B, C, and D to Human LEPR

Antibodies were generated according to Example 1 and tested for ability to bind human LEPR. Testing was done by way of an ELISA.

A 96 well high binding ELISA plate (Greiner Cat #650061) was coated with 50 μL/well human LEPR ECD (SEQ ID NO:30) at 2 μg/mL in carbonate buffer (50 mM NaHCO₃) at 4° C., overnight. The plate was washed with an automated plate washer and blocked for 1 hr at RT with 200 μL/well Casein (Thermo Fisher Cat #37528). The plate was then washed. Antibodies were titrated, starting at 50 μg/mL in Casein with 1:3 serial dilutions across the plate. Plate was incubated at RT for 1 hr. The plate is then washed.

Goat anti-human kappa AP conjugate (Southern Biotech Cat #2060-04) at 1 μg/mL in Casein was added to each well at 50 μL/well. The mixture was incubated at RT for 1 hr. The plate is then washed.

Develop with 100 μL/well PMP/AMP 0.5 mL in 15 mL water (developing reagent) and read at 560 nM.

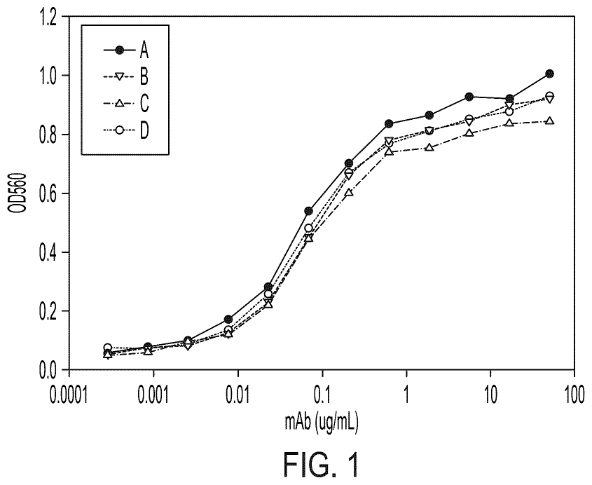

The data is depicted in FIG. 1. The curves indicate relative binding of IgG antibodies to human LEPR. The LEPR is bound to an ELISA plate and the antibodies are titrated in 3-fold increments starting at 50 μg/mL.

Summary: FIG. 1 demonstrates that Antibodies A, B, C, and D bind to human LEPR with varying affinities.

### Example 3

### Binding Affinity and Kinetics of Antibodies A, B, and C to LEPR of Various Species

Antibodies A, B, and C are IgG1 mAbs which bind leptin receptor (LEPR). The binding affinity and kinetics of antibodies A, B, and C were measured to human, cyno, mouse, rat, canine, and rabbit LEPR-ECD using surface plasmon resonance (SPR) on Biacore T200 (Cytiva). His-tagged ECDs were expressed and purified for each species and used for affinity analysis. A Protein A/G chip (Xantec, Cat #PAGHC200M) was utilized in this experiment with HBS-EP +0.01% BSA as running buffer. Antibodies were diluted to 1 μg/mL in running buffer and captured to a single flow cell on the chip surface. Each species of LEPR-ECD was diluted to 1000 nM in running buffer followed by 2-fold serial dilution. Each ligand concentration was injected over all flow cells at 100 L/min for 150 seconds followed by a 600 second dissociation phase. Regeneration was performed by injecting 10 mM glycine pH1.5 for 30 seconds at 10 μL/min. Data were analyzed in Biacore T200 Evaluation Software 3.1 by flow-cell 1 reference subtraction along with 0 nM blank subtraction. Data were fit globally using a “1:1 Binding” binding model to determine the on-rate (kₐ) and off-rate (k_{d}) for each ligand. The affinity (K_{D}) was calculated from the binding kinetics according to the relationship K_{D}=kₐ/k_{d}. Data reported as mean±standard deviation of experimental replicates.

TABLE 3
Binding affinity and kinetics of antibodies A, B, and C

| Species | Antibody | kₐ (1/Ms) | k_{d} (1/s) | K_{D} (M) |
|---|---|---|---|---|
| human | Antibody-A | 2.4E4 ± | 4.6E−4 ± | 2.0E−8 ± |
| | Mean ± SD (n = 3) | 1.9E3 | 5.8E−5 | 2.2E−9 |
| | Antibody-B | 1.1E4 ± | 2.3E−4 ± | 2.2E−8 ± |
| | Mean ± SD (n = 3) | 1.2E3 | 4.1E−5 | 6.8E−9 |
| | Antibody-C | 1.1E4 ± | 2.7E−4 ± | 2.6E−8 ± |
| | Mean ± SD (n = 3) | 1.4E3 | 3.4E−5 | 7.1E−9 |
| cyno | Antibody-A | 2.2E4 ± | 4.1E−4 ± | 1.9E−8 ± |
| | Mean ± SD (n = 3) | 7.2E2 | 3.9E−5 | 2.0E−9 |
| | Antibody-B | 1.5E4 ± | 4.0E−4 ± | 3.2E−8 ± |
| | Mean ± SD (n = 4) | 5.6E3 | 7.0E−5 | 1.7E−8 |
| | Antibody-C | 1.5E4 ± | 6.0E−4 ± | 4.7E−8 ± |
| | Mean ± SD (n = 4) | 5.9E3 | 1.2E−4 | 2.6E−8 |
| canine | Antibody-A | 9.2E3 ± | 1.2E−3 ± | 1.3E−7 ± |
| | Mean ± SD (n = 3) | 1.7E3 | 5.0E−5 | 2.4E−8 |
| | Antibody-B | 1.7E4 ± | 5.6E−3 ± | 3.3E−7 ± |
| | Mean ± SD (n = 2) | 1.2E4 | 3.6E−3 | 1.9E−8 |
| | Antibody-C | | | |
| mouse | Antibody-A (n = 3) | | | >1.0E−6 |
| | Antibody-B (n = 1) | | | >1.0E−6 |
| | Antibody-C (n = 1) | | | No Binding |
| rat | Antibody-A (n = 1) | | | No Binding |
| | Antibody-B (n = 1) | | | No Binding |
| | Antibody-C (n = 1) | | | No Binding |
| rabbit | Antibody-A (n = 1) | | | >1.0E−6 |
| | Antibody-B (n = 1) | | | >1.0E−6 |
| | Antibody-C (n = 1) | | | >1.0E−6 |

Summary: Table 3 demonstrates that Antibodies A, B, and C bind to human, cynomolgus monkey, and canine LEPR with varying affinities. Antibodies A, B, and C do not bind mouse, rat, nor rabbit LEPR with appreciable affinity.

### Example 4

### Anti-LEPR antibodies A, B, and C Activate LEPR of Several Species

LEPR is a cell surface single-pass transmembrane receptor. Activation of LEPR leads to phosphorylation of intracellular STAT3 (pSTAT3) and subsequent target gene transcription. To measure LEPR activation by anti-LEPR antibodies in the present disclosure, five independent STAT-luciferase reporter cell lines were established by introducing DNA plasmids encoding full-length LEPR of different species and STAT3-luciferase reporter gene into HEK293 cells (Table 4). These cell lines stably expressing full-length LEPR and STAT3-luciferase were maintained in DMEM medium (Gibco, #12430-054) with 10% fetal bovine serum (FBS, Gibco, #10082-147), antibiotics (Gibco, #15240-062), 1x Sodium Pyruvate (Gibco, #113060-070), 1x GlutaMax supplement (Gibco, #35050-061) and resistance selection drugs as indicated in Table 4.

Before plating the cells for luciferase assays, the poly-D-Lysine coated 96-well plates (Corning Inc, #354651) were rinsed once with phosphate buffered solution (PBS). Then, 20,000 to 24,000 STAT3-Luciferase reporter cells were seeded onto the poly-D-lysine 96-well plates in 100 μL/well plating medium consist of DMEM/F12 (3:1, Gibco, #93-0152DK), 10% fetal bovine serum (FBS, Gibco, #10082-147), 1x GlutaMax supplement (Gibco, #35050061) 20 mM HEPES (Gibco, #15630-080) and antibiotics (Gibco, 15240-062). After overnight culture at 37°° C. with 5% CO₂, the culture medium was changed to 75 μL assay medium containing OPTI-MEM (Gibco, #31985-070), 1% BSA (Gibco, BSA Fraction V (7.5%), #15260-037) and 0.1% dialyzed fetal bovine serum (FBS, Gibco, #26400-044). Test articles were diluted into 25 μL assay medium and were added to each well. Cells were further incubated for 20 hours at 37° C. with 5% CO₂. Cell lysing and One-Glo luciferase substrate solution (Promega, #E6120) were then added to each well, and relative luciferase units (RLUs) were measured on a ClarioStar plate reader. For each reporter cell line, positive control LEPR activation was calculated as the average RLUs stimulated by 20 nM recombinant human leptin protein (SEQ ID NO:27) minus the average RLUs of unstimulated cells and was set as maximum LEPR activation (100%). All test article induced LEPR activations were normalized to this positive control value of the same reporter cell line. The concentration response curves were then plotted as percentage of maximum LEPR activation to the function of log drug concentrations.

As shown in Table 5, the recombinant human leptin protein (SEQ ID NO:27) activated human, cynomolgus monkey, canine, rat, and mouse LEPR with EC₅₀ values of 0.0491±0.0044 nM, 0.6743±0.1098 nM, 0.5815±0.1723 nM, 6.293±0.6202 nM and 0.0788±0.017 nM, respectively. The anti-LEPR antibodies A, B, and C activated human LEPR with EC₅₀ values ranging from 0.1604 nM to 0.3576 nM and maximum LEPR activation ranging from 57.75% to 60.94% in HEK293-STAT3Luc-hLepR1 cells. The anti-LEPR antibodies A, B, and C also activated Macaca fascicularis LEPR (cyno-LEPR) with EC₅₀ values ranging from 0.8786 nM to 2.0187 nM and maximum LEPR activation of 20.00% to 21.30% in HEK293-STAT3Luc-cmLepR2 cells. In addition, anti-LEPR antibodies A, B, and C activated Canis lupus LEPR (canine-LEPR) with EC₅₀ values ranging from 0.4751 nM to 0.8543 nM and maximum LEPR activation of 76.47% to 76.70% in HEK293-STAT3Luc-dLepR7 cells. The anti-LEPR antibodies A, B, and C did not activate Rattus rattus LEPR (rat-LEPR) or Mus musculus LEPR (mouse-LEPR) at concentrations up to 90 nM.

TABLE 4
List of cell lines that were established
to test LEPR agonistic activities.

| | LEPR | STAT3- | |
|---|---|---|---|
| Reporter | GenBank | luciferase | |
| cell | ID, | GenBank | Drug resistance |
| line name | species | ID | selection |
| HEK293- | NP_002294.2, | JQ858512 | hygromycin (250 μg/mL), |
| STAT3Luc- | Homo sapiens | | Geneticin (800 μg/mL) |
| hLepR1 | | | |
| HEK293- | NP_001027991, | JQ858512 | hygromycin (250 μg/mL), |
| STAT3Luc- | Macaca | | Blasticidin (8 μg/mL) |
| cmLepR2 | fascicularis | | |
| HEK293- | XP038520011.1, | JQ858512 | hygromycin (250 μg/mL), |
| STAT3Luc- | Canis lupus | | Geneticin (800 μg/mL) |
| dLepR7 | | | |
| HEK293- | NP_036728.1, | JQ858512 | hygromycin (250 μg/mL), |
| STAT3Luc- | Rattus rattus | | puromycin (2 μg/mL) |
| ratLepR3 | | | |
| HEK293- | NP_666258.2, | JQ858512 | hygromycin (250 μg/mL), |
| STAT3Luc- | Mus musculus | | Zeocin (150 μg/mL) |
| msLepR9 | | | |

TABLE 5
Potency and Maximum activation of LEPRs by Antibodies.
Recombinant human leptin protein (SEQ ID NO: 27) was used
to generate dose response data and subsequent non-linear
fitted curves by Prizm software. The top of non-linear
fitted curve for each receptor species was compared to the
response to 20 nM recombinant human leptin protein (SEQ ID
NO: 27) and presented as percent agonism.

| | | human | | | | |
|---|---|---|---|---|---|---|
| | | leptin | Antibody-A | Antibody-B | Antibody-C | Antibody-CO |
| human- | EC₅₀ | 0.0491 ± | 0.1604 ± | 0.3576 ± | 0.2837 ± | 0.5651 ± |
| LEPR | (nM) ± | 0.0044 | 0.0193 | 0.0437 | 0.0421 | 0.0820 |
| | SEM | | | | | |
| | Agonism | 105.83 ± | 57.75 ± | 59.85 ± | 60.94 ± | 39.67 ± |
| | (%) ± | 2.4 | 2.61 | 3.37 | 1.59 | 1.16 |
| | SEM | | | | | |
| cyno- | EC₅₀ | 0.6743 ± | 0.8786 ± | 2.0187 ± | 1.4442 ± | 1.3038 ± |
| LEPR | (nM) ± | 0.1098 | 0.1839 | 0.3468 | 0.1828 | 0.0977 |
| | SEM | | | | | |
| | Agonism | 106.34 ± | 20.00 ± | 21.30 ± | 20.53 ± | 15.50 ± |
| | (%) ± | 4.84 | 3.41 | 4.21 | 5.21 | 3.95 |
| | SEM | | | | | |
| canine- | EC₅₀ | 0.5815 ± | 0.4751 ± | 0.8543 ± | 0.5091 ± | 50.2343 ± |
| LEPR | (nM) ± | 0.1723 | 0.0615 | 0.1891 | 0.0215 | 22.9733 |
| | SEM | | | | | |
| | Agonism | 107.5 ± | 76.47 ± | 76.63 ± | 76.70 ± | 42.47 ± |
| | (%) ± | 3.88 | 3.38 | 3.16 | 3.40 | 5.28 |
| | SEM | | | | | |
| rat- | EC₅₀ | 6.293 ± | inactive | inactive | inactive | inactive |
| LEPR | (nM) ± | 0.6202 | | | | |
| | SEM | | | | | |
| | Agonism | 106.1 ± | inactive | inactive | inactive | inactive |
| | (%) ± | 4.06 | | | | |
| | SEM | | | | | |
| mouse- | EC₅₀ | 0.0788 ± | inactive | inactive | inactive | inactive |
| LEPR | (nM) ± | 0.017 | | | | |
| | SEM | | | | | |
| | Agonism | 116.17 ± | inactive | inactive | inactive | inactive |
| | (%) ± | 5.78 | | | | |
| | SEM | | | | | |

Antibody CO is a comparative LEPR antibody, having HC sequence depicted as SEQ ID NO: 36 and LC sequence depicted as SEQ ID NO:37.

Summary: Antibodies A, B, and C activated human, cynomolgus monkey and canine LEPR in pSTAT3-Luciferease reporter assays. Antibodies A, B, and C did not activate mouse or rat LEPR.

### Example 5

### Antibodies A, B, and C Did Not Compete with Leptin to Bind to LEPR

To test whether the binding of anti-LEPR antibodies to LEPR will compete with leptin-LEPR interaction, an ELISA based competition binding assay was conducted.

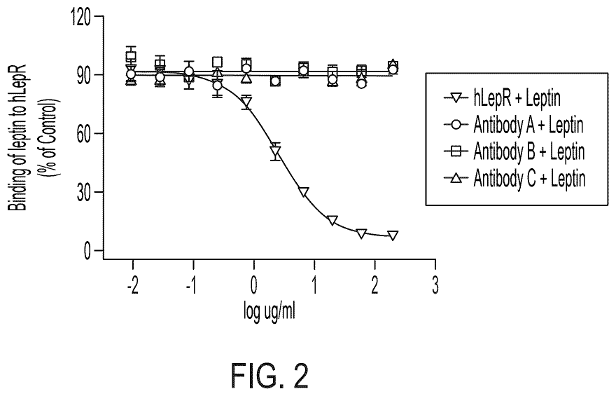

Recombinant LEPR extracellular domain fused to hlgG1-Fc (LEPR-Fc) (SEQ ID NO: 30) was diluted in Phosphate Buffered Solution (PBS, Gibco, #20012-027) and applied onto a 96-well Costar assay plate (Corning, #3690) at 5 μg/mL and 50 μL/well. The solution was incubated at 4° C. for 20 hours to allow sufficient protein coating. The plate was then rinsed 3 times with 160 μL/well of 1x wash buffer (R&D, #WA126, 25x), blocked for 1 hour at room temperature with Casein blocking solution (ThermoFisher, #37582), and rinsed by wash buffer 2 times. Serial concentrations of anti-LEPR antibodies (Antibodies A, B, and C) were then diluted in 60 μL of Casein blocking solution and added to each well. Serial concentrations of LEPR-Fc (SEQ ID NO:30) were used as positive control to compete with plate-bound receptor for leptin. Casein blocking solution was used as negative control. The plate was incubated with either anti-LEPR antibodies, positive or negative controls for 90 minutes at room temperature. Next, recombinant human leptin protein (R&D, #398-LP-05M) diluted in 20 μL Casein blocking buffer was added to each plate to a final concentration of 2 nM. The plate was then mixed for 30 seconds with gentle shaking followed by another 90 minutes of incubation at room temperature. The plate was rinsed 3 times with wash buffer. 0.5 μg/mL of Biotinylated anti-hLeptin antibody (Abcam, #AB271278) diluted in Casein blocking solution was added to each well at 50 μL/well and allowed to incubate at room temperature for 1 hour. Plate was then washed 3 times by wash buffer. Detection antibody Streptavidin-HRP (Life Technologies, #SNN2004) diluted 1:5,000 in Casein blocking solution was applied to each well at 50 μL/well and allowed to incubate at room temperature for 20 minutes. The plate was then washed 3 times and 50 μL of 1-Step-Ultra TMB-ELISA substrate (ThermoFischer, #34029) was then added to each well and incubated at room temperature for 5 minutes. Reaction was stopped by adding 25 μL of stop solution (R&D, #DY994) to each well. Data was obtained by a microplate reader with light absorbance set to 450 nm (OD450). Average OD450 readings in casein coated wells (without LEPR-Fc (SEQ ID NO: 30) coating) were subtracted from all raw OD450 readings to generate the normalized OD450 readings. The average normalized OD450 readings from negative controls (2 nM leptin incubated wells without any competing articles) was set as 100 percent (100%) binding. All normalized OD450 readings were compared to this OD450 reading and presented as percentages of binding. The top and bottom of dose response curve and IC₅₀ was calculated using GraphPad Prism software. As shown in Table 6 and FIG. 2, recombinant hLEPR-TEV-hIgG1Fc competed with Leptin to bind to LEPR on plate with an IC₅₀ of 2.575 μg/mL. None of the anti-LEPR antibodies (A, B, & C) competed with Leptin-LEPR interactions.

TABLE 6
Competitive binding assay of antibodies
and leptin protein to LEPR.

| | | | | hLEPR-TEV- |
|---|---|---|---|---|
| | Antibody-A | Antibody-B | Antibody-C | hIgG1Fc |
| Top (%) | 89.42 | Unstable | 90.01 | 92.03 |
| Bottom (%) | Unstable | 91.62 | 89.8 | 6.076 |
| Hill Slope | 14.32 | Unstable | Unstable | −1.07 |
| IC₅₀ (μg/mL) | N/A | N/A | N/A | 2.575 |

FIG. 2 shows a competitive binding assay of antibodies and leptin to LEPR, which shows binding of exemplary antibodies of the disclosure to hLepR, in the presence of leptin protein, as a percentage of control. Summary: Antibodies A, B, and C did not compete with recombinant leptin protein binding to its receptor LEPR in ELISA binding assay.

### Example 6

### Cell Surface LEPR Binding of Antibodies A, B, and C.

To test whether antibodies A, B, and C bind to human and cynomolgus monkey LEPR on live cell surface, flow cytometry was used to measure the cell surface bound antibodies with a fluorescently labeled secondary anti-human IgG. To ensure specificity of antibodies A, B, and C to bind LEPR each run included isotype IgG control, secondary only control, and 2 channel compensation controls. The aforementioned controls are well known in the art. For example, in the case of the IgG control, it is a non-binding IgG and contains Fc mutations that eliminate FcγR binding. The IgG control does not include YTE mutations.

The binding of antibodies A, B, and C to human LEPR and cynomolgus monkey LEPR on live cell surface was assessed in HEK293-STAT3Luc-hLepR1 and HEK293-STAT3Luc-cmLepR2 cells (Table 4 for cell lines used). Cells at exponential phase were dissociated by 1x Accutase™ (Invitrogen, #00-4555-56) according to manufacturer guidelines and strained via gravity flow on 40 μm cell strainers (Fisherbrand, #22363547). Cells were counted, pelleted, and resuspended in 20 μL per 0.2˜0.26^{e6} cells of blocking buffer including PBS (Gibco, #20012027), 0.3% BSA (Gibco, #15260-037), 4 mM EDTA, 0.01% Sodium Azide (Sigma, #S202-100G), 25 μg/mL human Fc Block (BD, #564219) and 100 μg/mL Goat Gamma Globulin (JacksonImmuno, #005-000-002). Cells were incubated in blocking buffer for 10 mins at room temperature. After one rinse with PBS, cells were transferred to U-bottom 96-well plate at 200 μL and 0.2˜0.26^{e6} cells/well. ArC™ Viability beads (Invitrogen, #A10346) and UltraComp eBeads Plus™ (ThermoFisher, #01-333-342) were added to assess cell viability and allow for compensation of fluorophores emission spectrum. Cells were pelleted and stained for 10 minutes at room temperature with 30 μL/well LIVE/DEAD™ Zombie Violet diluted 1:500 in PBS stain (BioLegend, #423113). Stained cells were washed with flow buffer that consisted of 1xPBS (Gibco, #20012027), 0.3% BSA (Gibco, #15260-037), 4 mM EDTA and 0.01% Sodium Azide (Sigma, #S202-100G). Cells were then incubated with 30 μL/well of antibodies diluted in flow buffer at 1000 nM as high dose followed by 9-point dose response including 5-fold dilutions not including 0 (totaling 9 concentrations for each Antibody) for 30 minutes at 4° C. in dark. After 3 rinses with flow buffer, cells were incubated for 20 minutes at 4° C. with 30 μL/well of AlexaFluor® 647-F (ab') 2 Goat Anti-Human IgG, Fcγ (JacksonImmuno, #109-606-170) 1:1000 diluted in cold flow buffer. Cells were resuspended in cold flow buffer at 120 μL/well and were subjected to flow cytometry on a BD Biosciences LSRFortessa X20 flow cytometer for acquisition in the following channels: FSC, SSC, BV421 (violet 405 nm 450/50) and APC/AF647 (red 633 nm 670/30) with 30,000 events per well limit. FlowJo v10 software was used to compensate and process the sample raw data then EC₅₀ were calculated for each Antibody utilizing GraphPad Prism non-linear regression asymmetric sigmoidal curve plotting concentration vs gMFI. This assay, which included both human and cynomolgus monkey LEPR cell lines, was run a total of 4 times on 4 different dates with averages shown in Table 7 and Table 8.

TABLE 7
Binding affinities of antibodies A, B, and
C to human LEPR on live cell surface.

| | EC₅₀ (nM) | SD | R² | Top (gMFI) | Bottom (gMFI) |
|---|---|---|---|---|---|
| IgG Isotype | 409 | 331 | 0.50 | 293 | 148 |
| control | | | | | |
| Antibody-A | 2.45 | 1.7 | 0.93 | 2461 | 131 |
| Antibody-B | 13.6 | 7.1 | 0.94 | 2940 | 123 |
| Antibody-C | 1.33 | 1.0 | 0.94 | 1850 | 144 |

TABLE 8
Binding affinities of antibodies A, B, and C to
cynomolgus monkey LEPR on live cell surface.

| | EC₅₀ (nM) | SD | R² | Top (gMFI) | Bottom (gMFI) |
|---|---|---|---|---|---|
| IgG Isotype | 727 | 0 | 0.60 | 131 | 69 |
| control | | | | | |
| Antibody-A | 0.74 | 0.7 | 0.95 | 12060 | 254 |
| Antibody-B | 1.11 | 1.0 | 0.95 | 12129 | 243 |
| Antibody-C | 0.83 | 0.6 | 0.95 | 10843 | 214 |

Summary: The data in table 7 and 8 demonstrate that Antibodies A, B, and C bind to human and cynomolgus monkey LEPR on cell surface with varying affinities.

### Example 7

### Binding of Antibody-A to Fcγ Receptors

To determine if the antibody Fc affects the binding characteristics of Antibody A to Fγy receptors, the binding to the human FcγRI, FcγRIIa, FcγRIIb, and FcγRIIIa receptor extracellular domains (ECDs) was measured by SPR at 25° C. Antibody A and Antibody A-IgG1 have the same CDRs. Antibody A differs from Antibody A-IgG1 by having L234A, L235A, D265S, M252Y, S254T, and T256E mutations in the heavy chain (HC). An IgG1 positive control and IgG1 non-binding control was used to confirm suitability of the assay.

Biacore T200 instrument (Cytiva) Biacore reagents and Scrubber2 Biacore Evaluation Software (Biologics 2008) were used for the SPR analysis of antibody binding. A Series-S CM5 chip (Cytiva P/N BR-1005-30) was prepared using the manufacturer's EDC/NHS amine coupling method (Cytiva P/N BR-1000-50). Briefly, the surfaces of all 4 flow cells were activated by injecting a 1:1 mixture of EDC/NHS for 7 minutes at 10 μL/minute. Protein A (Calbiochem P/N 539202) was diluted to 100 μg/mL in 10 mM acetate, pH 4.5 buffer and immobilized to approximately 4000 RU onto all 4 flow cells by 7 minute injection at a flow rate of 10 μL/minute. Un-reacted sites were blocked with a 7-minute injection of ethanolamine at 10 μL/minute. Injections of 2×10 μL of glycine pH 1.5 were used to remove any noncovalently associated protein.

The FcγR ECDs-FcγRI (CD64), FcγRIIA_131R, and FcγRIIA_131H (CD32a), FcγRIIIA_158V, FcγRIIIA_158F (CD16a), and FcγRIIb (CD32b; inhibitory receptor) (see e.g. Bruhns et al., Blood. 2009 Apr. 16; 113 (16): 3716-25) were produced from stable CHO cell expression according to methods well-known in the art and purified using IgG Sepharose and size exclusion chromatography.

For FcγRI binding, antibodies were diluted to 2.5 g/mL in running buffer (1x HBS-EP+, Teknova P/N H8022), and approximately 150 RU of each antibody was captured in flow cells 2 through 4 (RUcaptured). FC1 was the reference flow cell, therefore, no antibody was captured in FC1. FcγRI ECD was diluted to 200 nM in running buffer and then two-fold serially diluted in running buffer to 0.78 nM. Duplicate injections of each concentration were injected over all FCs at 40 μL/minute for 120 seconds followed by a 1200 second dissociation phase. Regeneration was performed by injecting 15 μL of 10 mM glycine pH 1.5 at 30 μL/minute over all FCs. Reference-subtracted data was collected as FC2-FC1, FC3-FC1, and FC4-FC1. The measurements were obtained at 25° C. The affinity (K_{D}) was calculated using either steady state equilibrium analysis with the Scrubber 2 Biacore® Evaluation Software or a “1:1 (Langmuir) binding” model in BIA Evaluation.

For FcγRIIa, FcγRIIb, and FcγRIIIa binding, antibodies were diluted to 5 μg/mL in running buffer, and approximately 500 RU is captured in flow cells 2 through 4 (RUcaptured). FC1 is the reference flow cell. Fcγ receptor ECDs were diluted to 10 μM in running buffer and then 2-fold serially diluted in running buffer to 39 nM. Duplicate injections of each concentration were injected over all FCs at 40 L/minute for 60 seconds followed by a 120 second dissociation phase. Regeneration was performed by injecting 15μL of 10 mM glycine pH 1.5 at 30 μL/minute over all FCs.

Reference-subtracted data was collected as FC2-FC1, FC3-FC1, and FC4-FC1. The measurements were obtained at 25° C. The affinity (K_{D}) was calculated using the steady state equilibrium analysis with the Scrubber 2 Biacore® Evaluation Software.

Following procedures essentially as described above, the following data as shown in Table 9 were obtained.

TABLE 9
In Vitro Binding Parameters of Antibody A to Human
Fcy Receptor ECDs Measured Using SPR at 25° C.

| Sample | Human Ligand | Average K_{D} | | Std Dev* |
|---|---|---|---|---|
| IgG1 positive control | FcγRI | 61.9 | pM | 1.3 |
| IgG1 non-binding control | FcγRI | No Binding | | |
| Antibody A-IgG1 | FcγRI | 67.6 | pM | 0.4 |
| Antibody A | FcγRI | No Binding | | |
| IgG1 positive control | FcγRIIA_131H | 0.52 | μM | 0.01 |
| IgG1 non-binding control | FcγRIIA_131H | No Binding | | |
| Antibody A-IgG1 | FcγRIIA_131H | 0.98 | μM | 0.03 |
| Antibody A | FcγRIIA_131H | No Binding | | |
| IgG1 positive control | FcγRIIA_131R | 0.53 | μM | 0.01 |
| IgG1 non-binding control | FcγRIIA_131R | >10 μM | | |
| Antibody A-IgG1 | FcγRIIA_131R | 1 | μM | 0.02 |
| Antibody A | FcγRIIA_131R | No Binding | | |
| IgG1 positive control | Fcγ RIIb | 2.21 | μM | 0.13 |
| IgG1 non-binding control | Fcγ RIIb | >10 μM | | |
| Antibody A-IgG1 | Fcγ RIIb | 3.61 | μM | 0.17 |
| Antibody A | Fcγ RIIb | No Binding | | |
| IgG1 positive control | Fcγ RIIIA_158V | 0.16 | μM | 0.01 |
| IgG1 non-binding control | Fcγ RIIIA_158V | >10 | | |
| Antibody A-IgG1 | Fcγ RIIIA_158V | 0.29 | μM | 0.03 |
| Antibody A | Fcγ RIIIA_158V | No Binding | | |
| IgG1 positive control | Fcγ RIIIA_158F | 0.86 | μM | 0.02 |
| IgG1 non-binding control | Fcγ RIIIA_158F | >10 μM | | |
| Antibody A-IgG1 | Fcγ RIIIA_158F | 1.56 | μM | 0.03 |

| | | |
|---|---|---|
| Antibody A | Fcγ RIIIA_158F | No Binding |

Assay was performed three independent times.
*Standard deviation was not determined for measurements >10 μM.

Summary: SPR binding data of Antibody A and various controls to FcγRI, FcγRIIA_131H, FcγRILA_131R, Fcγ RIIb, Fcγ RIIIA_158V, and Fcγ RIIIA_158F. Antibody A shows no detectable binding to Fcγ receptors.

### Example 8

### Antibodies A, B, and C Did Not De-Sensitize LEPR Signaling in Vitro

Activation of LEPR by leptin causes internalization of LEPR from cell surface. As consequence, LEPR becomes unavailable to extracellular ligand and irresponsive to subsequent ligand stimulation. The lack of response to repeated agonist exposure is referred to as receptor de-sensitization. To test whether the antibodies A, B, and C would de-sensitize LEPR, the phosphorylation of STAT3 protein in HEK293-STAT3Luc-hLepR1 cells was measured following an exposure, wash-out and re-exposure protocol.

HEK293-STAT3Luc-hLepR1 cells were seeded onto the poly-D-lysine 96-well plates (˜80,000 cells/well) with growth medium consist of DMEM (Gibco, #2430-054), 10% fetal bovine serum (FBS, Gibco, #10082-147), 1x Sodium Pyruvate (Gibco, #113060-070, 100x), 1x GlutaMAX Supplement (Gibco, #3Ag5050-061), and antibiotics (Gibco, #15240-062). After overnight culture at 37°° C. with 5% CO₂, the culture medium was removed and 50 μL/well serum-free medium (DMEM/F12, Gibco, #93-0152DK; 0.3% BSA, BSA Fraction V (7.5%), (Gibco, #15260-037) was added after 1 wash with serum-free medium. The cells were incubated for one hour in 50 μL of serum-free medium. The cells were then divided into control and antibody exposure groups. In negative control groups, 50 μL serum-free medium was added to each well. In antibody exposure groups, anti-LEPR antibodies were diluted in 50 μL of serum-free medium and added to the cells to reach a final concentration of 30 nM for each antibody. The 30 nM concentration is above EC99 concentration of every anti-LEPR antibody in the present disclosure, therefore cells were exposed to a saturating amount of anti-LEPR antibody and reached maximum LEPR activation by that antibody during the exposure. Cells in all groups were then incubated for one hour at 37° C. and 5% CO₂ to allow LEPR activation and potential internalization induced by anti-LEPR antibodies. After one hour incubation, antibody-and control-exposed cells were rinsed 3 times with 150 μL/well serum-free medium for 10 minutes (at 37° C., 5% CO₂) each time. Cells were then given growth medium (50 μL/well) and allowed to recover at 37° C. and 5% CO₂ for 3 hours. The growth medium was then replaced by serum-free medium (50 L/well) after one wash with serum-free medium and the cells were incubated at 37° C. and 5% CO₂ for one additional hour. Serial dilutions of each anti-LEPR antibody in the present disclosure was then added to the cells previously exposed to the same antibody or control-treated cells to allow LEPR activation by antibody re-exposure. After 15 minutes of incubation, the cells were lysed by 50 μL/well of lysis buffer (PerkinElmer, #ALSU-pST3-A10K) and mixed by shaking at room temperature for 10 minutes. Phosphorylated STAT3 (pSTAT3) was measured by AlphaLISA-SureFire-Ultra p-Stat (Tyr705) Assay kit (PerkinElmer, #ALSU-pST3-A10K) following manufacture's protocol. The average pSTAT3 Alpha-signal from cells treated by 30 nM recombinant human leptin protein (SEQ ID NO:27) minus the average pSTAT3 Alpha-signal of negative control was set as maximum STAT3 phosphorylation and defined as 100%. The pSTAT3 Alpha-signal readings of test article treatments were then normalized to the maximum STAT3 phosphorylation value. The concentration response curves were then plotted as percentage of pSTAT3 Alpha-signal to the function of log drug concentrations for each antibody.

As shown in Table 10, Anti-LEPR antibody treatments resulted in very similar concentration response curves in cells pre-exposed to antibodies compared to vehicle-exposed cells. Antibody A induced pSTAT3 signal in antibody A pre-exposed and washed-out cells with EC₅₀ of 0.3601 nM and maximum activation of 71.92%, compared to pSTAT3 induction by antibody A in vehicle-exposed cells with EC₅₀ of 0.3309 nM and maximum activation of 68.93%. Similarly, antibody B induced pSTAT3 signal in antibody B pre-exposed and washed-out cells with EC₅₀ of 1.057 nM and maximum activation of 77.58%, compared to pSTAT3 induction by antibody B in vehicle-exposed cells with EC₅₀ of 0.9862 nM and maximum activation of 70.48%. Finally, antibody C induced pSTAT3 signal in antibody C pre-exposed and washed-out cells with EC₅₀ of 1.136 nM and maximum activation of 73.63%, compared to pSTAT3 induction by antibody C in vehicle-exposed cells with EC of 1.038 nM and maximum activation of 71.69%.

TABLE 10
Activation of LEPR by antibodies after pre-exposure and wash-out

| | | Antibody | | Antibody | | Antibody |
|---|---|---|---|---|---|---|
| | | A after | | B after | | C after |
| | Antibody | pre- | Antibody | pre- | Antibody | pre- |
| | A | exposure | B | exposure | C | exposure |
| Top (%) | 68.93 | 71.92 | 70.48 | 77.58 | 71.69 | 73.63 |
| Bottom (%) | −0.9602 | −0.1807 | 0.3788 | 1.727 | −0.3473 | 0.03501 |
| Hill Slope | 1.28 | 1.376 | 1.436 | 1.318 | 1.258 | 1.215 |
| EC₅₀ (nM) | 0.3309 | 0.3601 | 0.9862 | 1.057 | 1.038 | 1.136 |

Summary: One-hour pre-exposure of LEPR-expressing HEK293 cells to 30 nM antibodies A, B, or C followed by 3-hour washout did not affect the subsequent concentration response curve of antibodies A, B, or C. These data showed that anti-LEPR antibodies A, B, and C did not de-sensitize human LEPR signaling pathway in vitro.

### Example 9

### Activation of Signaling Deficient LEPR by Antibodies A, B, and C

Missense mutation A409E in LEPR gene was associated with autosomal recessive early onset of obesity in human (Farooqi et al., 2007, N Engl J Med 356 (3): 237-247). Mutant human LEPR-A409E receptor failed to respond to leptin stimulation. To test whether the anti-LEPR antibodies can activate these mutant human LEPR receptors, the STAT3-Luciferase reporter assay was established using HEK293 cells that express hLEPR-A409E. These cells were cultured in growth medium DMEM/F12 (Gibco, #12430-054), 10% fetal bovine serum (FBS, Gibco, #10082-147), antibiotics (Gibco, #15240-062), Sodium Pyruvate (Gibco, #11360-070) and GlutaMax supplement (Gibco, #35050-061) in the presence of drug selection 250 μg/mL of Hygromycin (Invitrogen, #10687010) and 800 μg/mL of Geneticin (Corning, #30-234-C1).

Before plating the cells for luciferase assays, the poly-D-Lysine coated 96-well plates (Corning Inc, Bio Coat, #354651, Poly-D-Lysing coated) were rinsed once with phosphate buffered solution (PBS) and once by plating medium. Then, 20,000 to 24,000 STAT3-Luciferase reporter cells were seeded onto the poly-D-lysine 96-well plates in 100 μL/well plating medium consist of DMEM/F12 (3:1, Gibco, #93-0152DK), 10% fetal bovine serum (FBS, Gibco, #10082-147), 1x GlutaMax supplement (Gibco, #35050061) 20 mM HEPES (Gibco, #15630-080) and antibiotics (Gibco, 15240-062). After overnight culture at 37°° C. with 5% CO₂, the culture medium was changed to 75 μL assay medium containing OPTI-MEM (Gibco, #31985-070) with 1% BSA (BSA Fraction V (7.5%), Gibco, #15260-037), 0.1% dialyzed fetal bovine serum (FBS, Gibco, #26400-044) for 1 hour at 37° C. with 5% CO₂. Test articles were diluted into 25 μL assay medium and were added to each well. Cells were further incubated for 20 hours at 37° C. with 5% CO₂. Cell lysing and One-Glo luciferase substrate solution (Promega, #E6120) were then added to each well, and relative luciferase units (RLUs) were measured on a ClarioStar plate reader. The concentration response curves were then plotted as raw RLU readings to the function of log drug concentrations. EC₅₀ was calculated using GraphPad Prism software.

As shown in Table 11, antibodies A, B, and C activated mutant LEPR-A409E with EC₅₀ of 0.08811 nM, 0.272 nM and 0.2532 nM, respectively. By contrast, recombinant human leptin protein (SEQ ID NO:27) did not activate LEPR-A409E. These results suggest that the anti-LEPR antibodies in the present disclosure could be applied to treat patients with rare missense mutations in LEPR gene.

TABLE 11
Activation of mutant LEPR-A409E by antibodies.

| | Antibody-A | Antibody-B | Antibody-C | Leptin |
|---|---|---|---|---|
| Top (RLU) | 1395 | 1410 | 1421 | 259289* |
| Bottom (RLU) | 450 | 436.6 | 478.1 | 451.8 |
| Hill Slope | 1.899 | 1.406 | 1.698 | 0.2011 |
| EC₅₀ (nM) | 0.08811 | 0.272 | 0.2532 | 3.691E+19* |

*Top of curve and EC₅₀ for SEQ ID NO: 27 was projected by GraphPad Prism software. The high EC₅₀ value demonstrated that SEQ ID NO: 27 was inactive in this assay.
Summary: Antibodies A, B, and C activated signaling impaired mutant LEPR-A409E. By contrast, recombinant human leptin protein (SEQ NO: 27) did not activate the signaling deficient mutant LEPR-A409E.

### Example 10

### Antibodies A, B, and C Induced pERK in Human LEPR-Expressing HEK293 Cells

Antibodies were tested for the ability to induce phosphorylation of extracellular-signal-related kinase (pERK) in HEK293-STAT3Luc-hLepR1 cells. To measure pERK induction, cells were first cultured overnight at 37° C. in 96-well plate with DMEM medium supplemented with 10% FBS. On day 2, medium was removed, cells were washed in serum-free DMEM and incubated for an additional 24-hour in serum-free DMEM. On day 3, medium was removed and 1:4 series dilutions of antibodies A, B, and C with top concentration of 200nM in serum-free DMEM were added to the cells for 15 minutes. Testing was run in duplicates. Recombinant human leptin protein (SEQ ID NO:27) was used as a positive control in this assay. An IgGI with Fc modifications to ablate binding to Fcγ receptors was used as a negative control (IgG isotype control). Phospho-ERK1/2 was assessed in the whole cell lysate by measuring the amplified luminescence with AlphaLISA SureFire Ultra p-ERK1/2 (Thr202/Tyr204) kit (PerkinElmer, #ALSU-PERK-A-HV). Data was calculated using 4 parameters sigmoidal fit of the data (SigmaPlot software). Results, expressed in Table 12 are averaged EC₅₀ of two independent experiments.

TABLE 12
pERK induction by antibodies A, B, C, and Leptin.

| | | | | | IgG |
|---|---|---|---|---|---|
| | Antibody | Antibody | Antibody | | isotype |
| | A | B | C | Leptin | control |
| EC₅₀ | 1.22 + | 1.84 + | 0.82 ± | 1.075 ± | N/A |
| (nM) ± | 0.5 | 0.17 | 0.005 | 0.615 | |
| STDEV | | | | | |
| Maximum | 85995.5 | 83431.5 | 86973 | 137565 | 27300 |
| RLU | | | | | |
| Baseline | 29762.5 | 29762.5 | 29762.5 | 29762.5 | 29762.5 |
| RLU | | | | | |

Summary: Antibodies A, B, and C induced phosphorylation of ERK in human LEPR expressing HEK293 cells.

### Example 11

### Complement Component C1q Binding of Antibody-A by ELISA

A 96-well microplate was coated with 100 μL/well of each antibody diluted in DPBS (Dulbecco's HyClone) with a concentration range of 10 μg/mL to 0.19 μg/mL. Testing was performed in duplicate wells. The plate was sealed and incubated overnight at 4° C. The coating reagent was removed from each well and 200 μL/well of casein blocking reagent (Thermo) was added. The plate was sealed and incubated for 2 hours at room temperature (RT). Each well was washed 3 times with wash buffer (1xTBE with 0.05% Tween 20). One hundred microliters per well of Human Clq (MS Biomedical) at 10 μg/mL diluted in casein blocking reagent was added and incubated for 3 hours at RT. The plate was then washed three times with wash buffer before 100 L/well of a 1:800 dilution of Sheep anti-human C1q-HRP (Abcam #ab46191) in casein blocker was added and incubated for 1 hour at RT. The plate was washed 6 times with wash buffer, and 100 μL/well of TMB Substrate (Pierce) was added to each well and incubated for 7 minutes. One hundred microliters of 1 N HCl was added to each well to stop the reaction. Optical density was immediately measured using a colorimetric microplate reader set to 450 nm.

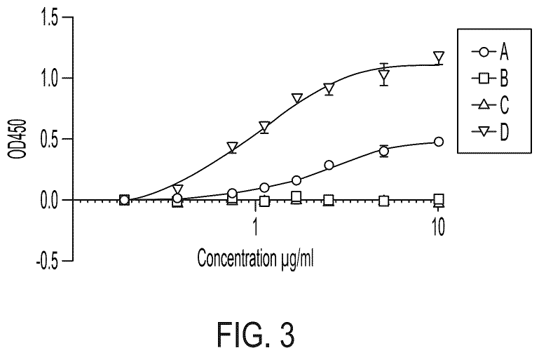

FIG. 3 shows C1q binding for Antibody A. Results (mean+SD) from 1 of 3 experiments performed are shown. (A) was an IgG1 isotype positive control and (B) was an IgG1 isotype non-binding control used to confirm suitability of the assay. (C) was Antibody A and (D) was Antibody A-IgG1 control. Curve Fit: 4-Parameter Logistic in GraphPad Prism. Conditions B and C are overlapping and essentially are at baseline.

Summary: Antibody A does not bind complement component Clq by ELISA. Relevant control molecules and Antibody A-IgG1 worked as expected.

### Example 12

### Relative FcRn Binding of Antibody-A by FcRn-HPLC

IgG binding to neonatal Fc receptor (FcRn) is a key mechanism for antibody recycling and a determinant of antibody pharmacokinetics (PK). Modification of an antibody's Fc region has been shown to modulate antibody PK. Antibody-A is an IgG1 mAb with L234A, L235A, D265S mutations to reduce FcγR and Clq binding and M252Y, S254T, and T256E mutations to modulate the antibody's clearance profile. The impact of these combined mutation sets was assessed by FcRn-HPLC. Stock antibody solutions were diluted to 1 mg/mL into 20 mM MES, 140 mM NaCl, pH5.5. An Agilent 1290 HPLC equipped with an FcRn Affinity Column Gen2 (Roche, Cat #09430857001) was pre-equilibrated with 20 mM MES, 140 mM NaCl, pH5.5 with a flow rate of 0.2mL/min. 20 μμL antibody injections were made followed by 10 minute column wash with 20 mM MES, 140 mM NaCl, pH5.5. Antibodies were then eluted by pH gradient elution into 20 mM Tris, 140 mM NaCl, pH8.8 over 90 minutes. Chromatograms were blank-subtracted to remove baseline drift. Main peak retention time and peak width at 50% peak height are shown in Table 13.

Briakinumab is an IgG1 control with impaired PK due to ineffective FcRn recycling whereas Ustekinumab displays an FcRn profile typical of IgGs. (Schoch A et al. Charge-mediated influence of the antibody variable domain on FcRn-dependent pharmacokinetics. Proc Natl Acad Sci U S A. 2015 May 12;112 (19): 5997-6002.) A panel of comparator antibodies with matched VH and VL domains were generated for this study, only differing in their Fc region: 1) Antibody-A with non-modified IgG1 (Antibody A IgG1) 2) Antibody A with M252Y, S254T, and T256E but without L234A, L235A, D265S (Antibody A IgG1-YTE) 3) Antibody A grafted onto an IgG4 S228P, F234A, L235A (Antibody A IgG4PAA).

Ustekinumab displayed a relatively sharp peak with 69.4 minute retention time and 1.9 minute peak width whereas Briakinumab eluted with a broad 10 minute peak width and high retention time at 88.9 minutes. FcRn binding profile of Antibody A IgG1 was highly similar to Ustekinumab with nearly identical retention time and peak width. All YTE containing samples displayed similar FcRn binding profiles with increased elution pH (retention time) and moderate broadening of peak widths relative to Antibody A IgG1.

TABLE 13
FcRn Binding Profile by FcRn-HPLC

| | Retention Time | Peak Width |
|---|---|---|
| Antibody | (minutes) | (minutes) |
| Ustekinumab | 69.4 | 1.9 |
| Briakinumab | 88.9 | 10.0 |
| Antibody A | 80.1 | 2.7 |
| Antibody A IgG1 | 69.5 | 1.7 |
| Antibody A IgG1-YTE | 80.3 | 2.8 |
| Antibody A IgG4PAA-YTE | 79.6 | 2.7 |

Summary: Antibody binding to FcRn was tested by FcRn-HPLC. Antibody A eluted the FcRn-HPLC column with a 2.7 minute peak width and a retention time delayed relative to its variable domain matched IgG1 control and relative to Ustekinumab.

### Example 14

### LEPR Aagonist and GLP-1 Aagonist Combination Treatment Led to Significant Body Weight Loss in Diet Induced Obese Mice

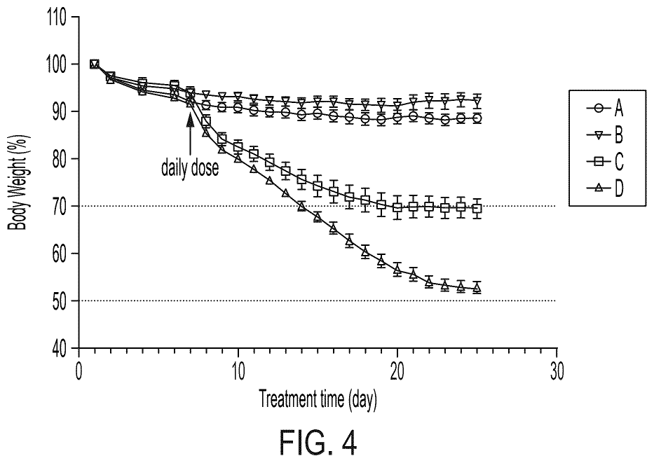

Four weeks old male black 6 (BL6) mice were fed high fat diet (Research Diet #D12492) for 16 weeks. These 20-week-old diet induced obese (DIO) male mice were used for the study described below. Antibody A, B, C, and D of the present disclosure are not cross-reactive in mice. A leptin receptor agonist including leptin protein fused to an Fc (Fc-leptin) (SEQ ID NO:38) is used as a surrogate for LEPR agonist antibody. SEQ ID NO:38 binds and agonizes the leptin receptor. At the beginning of the study (day 1), the diet was changed to normal chow diet (Research Diets #D2014). On day 7, mice received daily subcutaneous injections of vehicle (40 mM Tris, pH 8.0) (FIG. 4, condition A), LEPR agonist (Fc-leptin (SEQ ID NO:38)) at 40 nmol/kg (FIG. 4, condition B), GLP-1 receptor agonist (SEQ ID NO:52) at 30 nmol/kg (FIG. 4, condition C), combination of LEPR agonist (SEQ ID NO: 38) (40 nmol/kg) +GLP-1 receptor agonist (SEQ ID NO:52) (30 nmol/kg) (FIG. 4, condition D). As shown in FIG. 4, combination treatment of leptin receptor agonist and a GLP-1 receptor agonist (SEQ ID NO:52) resulted in additional body weight loss compared to treatment with GLP-1 receptor agonist alone or LEPR agonist (SEQ ID NO:38) treatment alone.

FIG. 4 shows treatment of GLP-1 receptor agonist in combination with leptin receptor agonist led to additional weight loss in obese mice. The diet induced obese mice were placed on a diet switch from high fat diet (HFD) to normal chow for 6 days. These mice were then subject to daily subcutaneous injections of compounds starting at day 7, indicated by the arrow. Body weight was measured daily. Percentage body weight changes from baseline were plotted against time and presented in the graphs. Conditions include (A) vehicle control; (B) leptin receptor agonist (SEQ ID NO:38); (C) GLP-1 receptor agonist (SEQ ID NO:52); (D) GLP-1 receptor agonist (SEQ ID NO:52) and leptin receptor agonist (SEQ ID NO:38) combination.

### Example 15

### LEPR Agonist and GLP1R/GIPR Dual Agonist Combination Treatment Led to Significant Body Weight Loss in Diet Induced Obese Mice

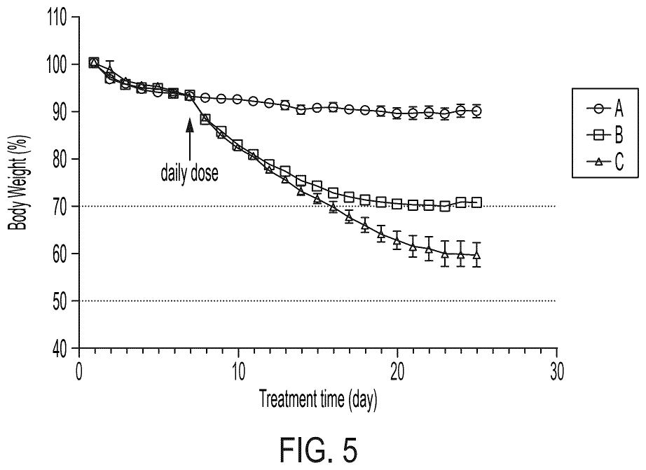

Four weeks old male black 6 (BL6) mice were fed high fat diet (Research Diet #D12492) for 16 weeks. These 20-week-old diet induced obese (DIO) male mice were used for the study described below. At the beginning of the study (day 1), the diet was changed to normal chow diet (Research Diets #D2014). On day 7, mice received daily subcutaneous injections of vehicle (40 mM Tris, pH 8.0) (FIG. 5, condition A), GLP1R/GIPR dual agonist (SEQ ID NO:53) at 3 nmol/kg (FIG. 5, condition B), combination of leptin receptor agonist (SEQ ID NO:38) (40 nmol/kg) +GLP1R/GIPR dual agonist (3 nmol/kg) (FIG. 5, condition C). As shown in FIG. 5, combination treatment of a leptin receptor agonist and GLP1R/GIPR dual agonist resulted in additional body weight loss compared to GLP1R/GIPR dual agonist treatment alone.

FIG. 5 shows treatment of dual GLP1R/GIPR agonist in combination with a leptin receptor agonist led to additional weight loss in obese mice. The diet induced obese mice were placed on a diet switch from high fat diet (HFD) to normal chow for 6 days. These mice were then subject to daily subcutaneous injections of compounds starting at day 7, indicated by the arrow. Body weight was measured daily. Percentage body weight changes from baseline were plotted against time and presented in the graphs. Conditions include (A) vehicle control; (B) dual GLP1R/GIPR agonist (SEQ ID NO:53); (C) dual GLP1R/GIPR agonist (SEQ ID NO:53) and leptin receptor agonist (SEQ ID NO:38) combination.

### Example 16. LEPR Agonist and GLP1R/GIPR/GCGR Triple Agonist Combination Treatment Led to Significant Body Weight Loss in Diet Induced Obese Mice

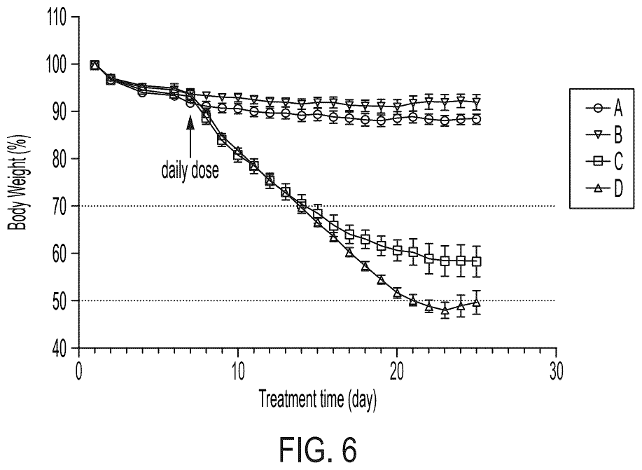

Four weeks old male black 6 (BL6) mice were fed high fat diet (Research Diet #D12492) for 16 weeks. These 20-week-old diet induced obese (DIO) male mice were used for the study described below. At the beginning of the study (day 1), the diet was changed to normal chow diet (Research Diets #D2014). On day 7, mice received daily subcutaneous injections of vehicle (40 mM Tris, pH 8.0) (FIG. 6, condition A), leptin receptor agonist (SEQ ID NO:38) at 40nmol/kg (FIG. 6, condition B), GLP1R/GIPR/GCGR triple agonist (SEQ ID NO:55) at 3nmol/kg (FIG. 6, condition C), combination of leptin receptor agonist (40nmol/kg) +GLP1R/GIPR/GCGR triple agonist (3nmol/kg) (FIG. 6, condition D). As shown in FIG. 6, combination treatment of leptin receptor agonist and GLP1R/GIPR/GCGR triple agonist resulted in additional body weight loss compared to GLP1R/GIPR/GCGR triple agonist treatment alone or leptin receptor agonist alone treatment alone.

FIG. 6 shows treatment of GLP1R/GIPR/GCGR triple agonist in combination with leptin receptor agonist led to additional weight loss in obese mice. The diet induced obese mice were placed on a diet switch from high fat diet (HFD) to normal chow for 6 days. These mice were then subject to daily subcutaneous injections of compounds starting at day 7, indicated by the arrow. Body weight was measured daily. Percentage body weight changes from baseline were plotted against time and presented in the graphs. Compounds include (A) vehicle control; (B) leptin receptor agonist (SEQ ID NO:38); (C) GLP1R/GIPR/GCGR triple agonist (SEQ ID NO:55); (D) GLP1R/GIPR/GCGR triple agonist (SEQ ID NO:55) and leptin receptor agonist (SEQ ID NO:38) combination.

### Example 17

### LEPR Agonist and Glucagon Receptor Agonist Combination Treatment Led to Significant Body Weight Loss in Diet Induced Obese Mice

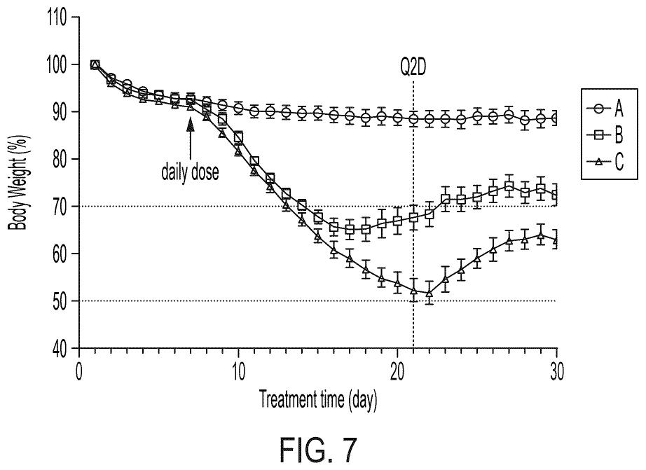

Four weeks old male black 6 (BL6) mice were fed high fat diet (Research Diet #D12492) for 16 weeks. These 20-week-old diet induced obese (DIO) male mice were used for the study described below. At the beginning of the study (day 1), the diet was changed to normal chow diet (Research Diets #D2014). On day 7, mice received daily subcutaneous injections of vehicle (40 mM Tris, pH 8.0) (FIG. 7, condition A), glucagon receptor agonist (SEQ ID NO:57) at 6 nmol/kg (FIG. 7, condition B), combination of leptin receptor agonist (40 nmol/kg) +glucagon receptor agonist (6nmol/kg) (FIG. 7, condition C). At day 21, mice were switched to receiving subcutaneous compound dosing every other day (Q2D) until the end of the study. As shown in FIG. 7, combination treatment of leptin receptor agonist and glucagon receptor agonist resulted in additional body weight loss compared to glucagon receptor agonist treatment alone.

FIG. 7 shows treatment of glucagon receptor agonist in combination with leptin receptor agonist led to additional weight loss in obese mice. The diet induced obese mice were placed on a diet switch from high fat diet (HFD) to normal chow for 6 days. These mice were then subject to daily subcutaneous injections of compounds starting at day 7, indicated by the arrow. Mice were switched to receiving subcutaneous compound injections every other day starting at day 21 (Q2D). Body weight was measured daily. Percentage body weight changes from baseline were plotted against time and presented in the graphs. Conditions include (A) vehicle control; (B) glucagon receptor agonist (SEQ ID NO:57); (C) glucagon receptor agonist (SEQ ID NO:57) and leptin receptor agonist (SEQ ID NO:38) combination.

Summary: When a surrogate LEPR agonist to the antibodies of the present disclosure is combined with either an agent having GLP-1 receptor agonist activity, an agent having GLP1/GIP receptor dual agonist activity, or an agent having GLP1/GIP/Glucagon receptor: triple agonist activity, significantly more weight loss was observed in DIO mice receiving combination treatments than treatment with a single agent. In addition, a leptin receptor agonist in combination with glucagon receptor agonist caused significant more weight loss than glucagon receptor agonist alone in DIO mice.

### Example 18

### Amylin Receptor Agonist and LEPR Agonist Combination Treatment Led to Significant Body Weight Loss in Diet Induced Obese Rats

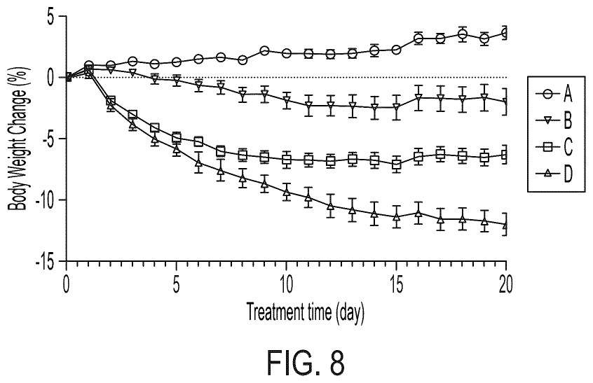

Eight weeks old male Long Evans rats were fed with high fat diet (Teklad Custom Diet #95217) for 12 weeks. The resulting 20-week-old diet-induced obese (DIO) rats were used for the study. These DIO rats were then given daily subcutaneous injections of an amylin receptor agonist (SEQ ID NO:39) (10 nmol/kg), LEPR agonist (SEQ ID NO:38) (100 nmol/kg), combination (sequential administration) of the amylin receptor agonist (SEQ ID NO: 39) (10 nmol/kg) and LEPR agonist (100 nmol/kg), or placebo. Body weight of these DIO rats were measured daily. As shown FIG. 8, combination treatment with amylin receptor agonist and leptin receptor agonist resulted in significantly more weight loss compared to either single agent treatment alone.

FIG. 8 shows treatment with amylin receptor agonist (SEQ ID NO:39) in combination with a leptin receptor agonist (SEQ ID NO:38) led to additional weight loss in diet induced obese rats. High fat diet fed obese rats were given daily subcutaneous injections of drugs as indicated in the graph legend. Body weight was measured daily. Percentage body weight changes from baseline were plotted against time and presented in the graphs. Conditions include (A) vehicle control; (B) leptin receptor agonist (SEQ ID NO:38) at 100nmol/kg; (C) amylin receptor agonist (SEQ ID NO:39) at 10 nmol/kg; and (D) combination of 10 nmol/kg amylin receptor agonist (SEQ ID NO:39) and 100 nmol/kg of a leptin receptor agonist (SEQ ID NO:38).

Summary: Combination treatment of leptin receptor agonist and an amylin receptor agonist caused more weight loss than either single agent treatment alone in DIO rats.

### Example 19

### LEPR Agonist Combined with Either Amylin Receptor Agonist or Amylin and Calcitonin Receptor Co-Agonist Led to Significant Body Weight Loss in Diet Induced Obese Rats

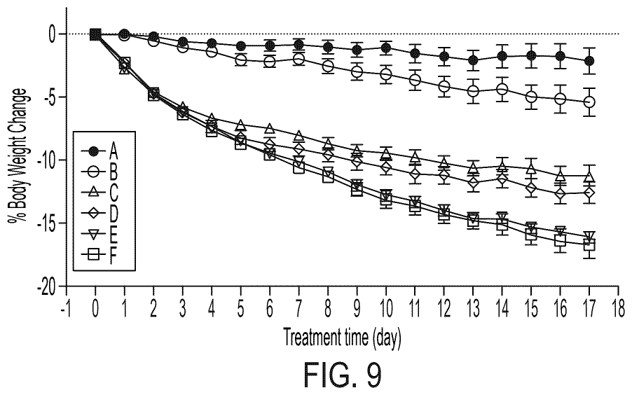

Eight weeks old male Long Evans rats were fed with high fat diet (Teklad Custom Diet #95217) for 14 weeks. The resulting 22-week-old diet-induced obese (DIO) rats were used for the study. These DIO rats were then given daily subcutaneous injections of an amylin receptor agonist (SEQ ID NO: 41) (6 nmol/kg), an amylin and calcitonin receptor co-agonist (SEQ ID NO: 51) (5 nmol/kg), a LEPR agonist (SEQ ID NO: 38) (90 nmol/kg), or their combinations. Body weight of these DIO rats were measured daily. As shown FIG. 9, combination treatment with amylin receptor agonist and leptin receptor agonist resulted in significantly more weight loss compared to either single agent treatment alone. Similarly, combination treatment with amylin receptor and calcitonin receptor co-agonist and leptin receptor agonist also led to significantly more weight loss compared to either single agent treatment alone.

FIG. 9 shows treatment with leptin receptor agonist (SEQ ID NO:38) in combination with either amylin receptor agonist (SEQ ID NO: 41) or amylin and calcitonin receptor co-agonist (SEQ ID NO: 51) led to additional weight loss in diet induced obese rats. High fat diet fed obese rats were given daily subcutaneous injections of drugs as indicated in the graph legend. Body weight was measured daily. Percentage body weight changes from baseline were plotted against time and presented in the graphs. Conditions include (A) vehicle control; (B) leptin receptor agonist (SEQ ID NO:38); (C) amylin receptor agonist (SEQ ID NO: 41); (D) amylin and calcitonin receptor co-agonist (SEQ ID NO: 51); (E) combination of leptin receptor agonist (SEQ ID NO:38) and amylin receptor agonist (SEQ ID NO: 41); (F) combination of leptin receptor agonist (SEQ ID NO:38) and amylin and calcitonin receptor co-agonist (SEQ ID NO: 51).

Summary: Combination treatment of leptin receptor agonist with either amylin receptor agonist or amylin and calcitonin receptor co-agonist caused more weight loss than single agent treatment alone in DIO rats.

### Example 20

### Treatment of Monkeys with Antibody A Led to Weight Loss

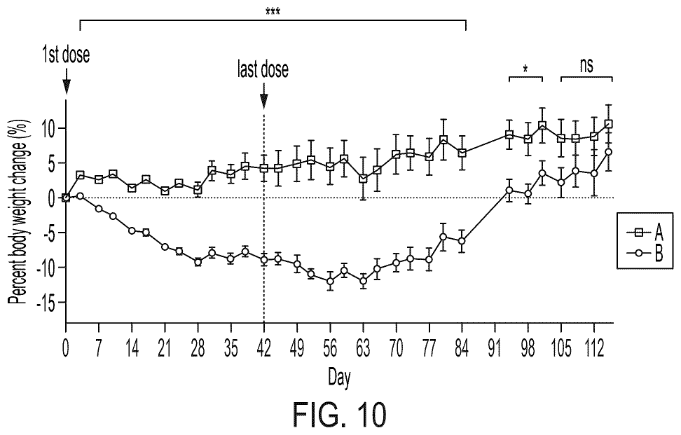

The efficacy of Antibody A was tested in non-human primates. Fourteen male 2˜5 years old non-obese cynomolgus (Macaca fascicularis) monkeys weighing 3˜5 kg were group housed in European caging (7 monkeys per cage) for 45 days. Monkeys were then randomized according to their body weight into two groups receiving subcutaneous administrations of either Antibody A (10 mg/kg, N=7) or placebo (phosphate buffered saline, N=7) once a week for 6 weeks. Monkeys were provided meals (Purina 4059) twice a day and ad libitum access to water for the entire duration of the study. Body weight was measured twice a week during the dosing period and three additional weeks after the final dose. Percent body weight change was plotted as a function of time. As shown in FIG. 10 and table 14, monkeys that received antibody A treatment lost significant amount of weight compared to monkeys received placebo.

FIG. 10 shows Antibody A treatment led to significant weight loss in Cynomolgus monkeys. Male monkeys (Macaca fascicularis) received once weekly subcutaneous administrations of placebo (A) or 10 mg/kg Antibody A (B) for 6 weeks. Body weights were measured for a total of 115 days. Average percent body weight change from baseline at day 0 was plotted at each time point. Error bars represent standard error of mean (SEM). Antibody A treated monkeys showed significant body weight loss compared to placebo group for up to day 101 of the study. *P<0.005, *P<0.05, ns: P>0.05 by multiple unpaired t-test at all time points except for day 0 compared to placebo group.

TABLE 14
Percentage weight change from baseline and difference
between Antibody A and placebo group

| | Percent body weight change | | Average difference (%) |
|---|---|---|---|
| Treatment | from baseline (Average % + SEM) | | between Antibody A and |
| day | Antibody A group | Vehicle group | vehicle groups |
| 0 | 0 ± 0 | 0 ± 0 | 0.00 |
| 3 | 0.27 ± 0.34 | 3.29 ± 0.39 | −3.03 |
| 7 | −1.52 ± 0.43 | 2.63 ± 0.56 | −4.15 |
| 10 | −2.6 ± 0.39 | 3.53 ± 0.40 | −6.13 |
| 14 | −4.65 ± 0.43 | 1.40 ± 0.23 | −6.06 |
| 17 | −5.01 ± 0.53 | 2.70 ± 0.39 | −7.71 |
| 21 | −7.00 ± 0.49 | 1.02 ± 0.27 | −8.02 |
| 24 | −7.70 ± 0.50 | 2.17 ± 0.44 | −9.86 |
| 28 | −9.21 ± 0.59 | 1.21 ± 1.13 | −10.42 |
| 31 | −7.91 ± 0.82 | 3.89 ± 1.42 | −11.80 |
| 35 | −8.68 ± 0.80 | 3.44 ± 1.39 | −12.12 |
| 38 | −7.71 ± 0.77 | 4.55 ± 1.95 | −12.26 |
| 42 | −8.86 ± 0.79 | 4.24 ± 1.88 | −13.10 |
| 45 | −8.75 ± 0.85 | 4.23 ± 2.55 | −12.98 |
| 49 | −9.45 ± 1.21 | 4.94 ± 2.55 | −14.39 |
| 52 | −10.92 ± 0.74 | 5.42 ± 2.82 | −16.34 |
| 56 | −11.98 ± 1.39 | 4.56 ± 2.67 | −16.55 |
| 59 | −10.41 ± 1.04 | 5.67 ± 2.64 | −16.08 |
| 63 | −11.93 ± 1.06 | 2.81 ± 3.12 | −14.74 |
| 66 | −10.21 ± 1.49 | 3.97 ± 3.10 | −14.18 |
| 70 | −9.345 ± 1.29 | 6.29 ± 2.85 | −15.63 |
| 73 | −8.72 ± 1.67 | 6.48 ± 2.51 | −15.20 |
| 77 | −8.84 ± 1.64 | 5.96 ± 2.63 | −14.79 |
| 80 | −5.65 ± 2.00 | 8.34 ± 2.93 | −13.98 |
| 84 | −6.25 ± 1.65 | 6.50 ± 2.46 | −12.75 |
| 94 | 0.76 ± 1.88 | 9.12 ± 2.10 | −8.36 |
| 98 | 0.30 ± 1.69 | 8.48 ± 2.34 | −8.18 |
| 101 | 3.28 ± 2.06 | 10.4 ± 2.52 | −7.13 |
| 105 | 1.52 ± 2.45 | 8.60 ± 2.67 | −7.08 |
| 108 | 3.45 ± 2.72 | 8.59 ± 2.47 | −5.14 |
| 112 | 3.59 ± 3.34 | 8.89 ± 2.71 | −5.30 |
| 115 | 6.19 ± 3.17 | 10.62 ± 2.77 | −4.42 |

Summary: Antibody A treatment led to significant weight loss compared to placebo in non-obese Cynomolgus monkeys.

SEQUENCE LISTING
SEQ ID NO: 1 LCDR1 of Antibodies A, C, and D
RASQGISSSLA
SEQ ID NO: 2 LCDR1 of Antibody B
RVSQGISSSLA
SEQ ID NO: 3: LCDR2 of Antibodies A, B, C, and D
YTASTLQS
SEQ ID NO: 4: LCDR3 of Antibody A
QQLIYYPFT
SEQ ID NO: 5 LCDR3 of Antibodies B and D
QQLNYYPFT
SEQ ID NO: 6: LCDR3 of Antibody C
QQLNYYPFS
SEQ ID NO: 7 HCDR1 of Antibodies A, B, C, and D
AASGFTFSSFAMS
SEQ ID NO: 8 HCDR2 of Antibodies A, B, C, and D
AISGSGGSTY
SEQ ID NO: 9 HCDR3 of Antibodies A, B, C, and D
AKDQGDWDFLFDY
SEQ ID NO: 10 VL of Antibody A
DIQMTQSPSFLSASVGDRVTITCRASQGISSSLAWYQQKPGKAPKLLIYTASTLQSGV
PSRFSGSGSGTEFTLTISSLQPEDFATYYCQQLIYYPFTFGQGTKVEIK
SEQ ID NO: 11 LC of Antibody A
DIQMTQSPSFLSASVGDRVTITCRASQGISSSLAWYQQKPGKAPKLLIYTASTLQSGV
PSRFSGSGSGTEFTLTISSLQPEDFATYYCQQLIYYPFTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO: 12 VL of Antibody B
DIQMTQSPSFLSASVGDRVTITCRVSQGISSSLAWYQQKPGKAPKLLIYTASTLQSGV
PSRFSGSGSGTEFTLTISSLQPEDFATYYCQQLNYYPFTFGQGTKVEIK
SEQ ID NO: 13 LC of Antibody B
DIQMTQSPSFLSASVGDRVTITCRVSQGISSSLAWYQQKPGKAPKLLIYTASTLOSGV
PSRFSGSGSGTEFTLTISSLQPEDFATYYCQQLNYYPFTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO: 14 VL of Antibody C
DIQMTQSPSFLSASVGDRVTITCRASQGISSSLAWYQQKPGKAPKLLIYTASTLQSGV
PSRFSGSGSGTEFTLTISSLQPEDFATYYCQQLNYYPFSFGQGTKVEIK
SEQ ID NO: 15 LC of Antibody C
DIQMTQSPSFLSASVGDRVTITCRASQGISSSLAWYQQKPGKAPKLLIYTASTLOSGV
PSRFSGSGSGTEFTLTISSLQPEDFATYYCQQLNYYPFSFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO: 16; Antibody D VL
DIQMTQSPSFLSASVGDRVTITCRASQGISSSLAWYQQKPGKAPKLLIYTASTLQSGV
PSRFSGSGSGTEFTLTISSLQPEDFATYYCQQLNYYPFTFGQGTKVEIK
SEQ ID NO: 17; Antibody D LC
DIQMTQSPSFLSASVGDRVTITCRASQGISSSLAWYQQKPGKAPKLLIYTASTLQSGV
PSRFSGSGSGTEFTLTISSLQPEDFATYYCQQLNYYPFTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO: 18 VH of Antibody A, B, C, and D
EVQLLESGGGLVQPGGSLRLSCAASGFTFSSFAMSWVRQAPGKGLEWVSAISGSGGS
TYSADSVRGRFTISRDNSKNTLYLQMNSLRAEDTAVYYCAKDQGDWDFLFDYWGQ
GTLVTVSS
SEQ ID NO: 19 HC of Antibody A, B, C, and D
EVQLLESGGGLVQPGGSLRLSCAASGFTFSSFAMSWVRQAPGKGLEWVSAISGSGGS
TYSADSVRGRFTISRDNSKNTLYLQMNSLRAEDTAVYYCAKDQGDWDFLFDYWGQ
GTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRVEPKSCDKT
HTCPPCPAPEAAGGPSVFLFPPKPKDTLYITREPEVTCVVVSVSHEDPEVKFNWYVD
GVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 20 Signal Peptide Sequence
METDTLLLWVLLLWVPGSTG
SEQ ID NO: 21; Signal Peptide cDNA
ATGGAGACAGACACACTCCTGCTATGGGTACTGCTGCTCTGGGTTCCAGGATCTA
CCGGT
SEQ ID NO: 22 LC A, cDNA
GACATCCAGATGACCCAGTCTCCATCCTTCCTGTCTGCATCTGTGGGAGACAGAG
TCACCATCACTTGCCGGGCCAGTCAGGGCATTAGCAGTTCTTTAGCCTGGTATCA
GCAAAAACCAGGAAAAGCCCCTAAGCTCCTGATCTATACTGCATCCACTTTGCAA
AGTGGGGTCCCATCAAGGTTCAGCGGCAGTGGATCTGGGACAGAATTCACTCTC
ACAATCAGCAGCCTGCAGCCTGAAGATTTTGCAACTTATTACTGTCAACAGCTTA
TTTATTACCCTTTCACTTTCGGCCAGGGGACCAAGGTGGAAATCAAAAGAACTGT
GGCGGCGCCATCTGTCTTCATCTTCCCGCCATCTGATGAGCAGTTGAAATCCGGA
ACTGCCTCTGTTGTGTGCCTGCTGAATAACTTCTATCCCAGAGAGGCCAAAGTAC
AGTGGAAGGTGGATAACGCCCTCCAATCGGGTAACTCCCAGGAGAGTGTCACAG
AGCAGGACAGCAAGGACAGCACCTACAGCCTCAGCAGCACCCTGACGCTGAGCA
AAGCAGACTACGAGAAACACAAAGTCTACGCCTGCGAAGTCACCCATCAGGGCC
TGAGCTCGCCCGTCACAAAGAGCTTCAACAGGGGAGAGTGC
SEQ ID NO: 23 LC B, cDNA
GACATCCAGATGACCCAGTCTCCATCCTTCCTGTCTGCATCTGTGGGAGACAGAG
TCACCATCACTTGCCGGGTCAGTCAGGGCATTAGCAGTTCTTTAGCCTGGTATCA
GCAAAAACCAGGAAAAGCCCCTAAGCTCCTGATCTATACTGCATCCACTTTGCAA
AGTGGGGTCCCATCAAGGTTCAGCGGCAGTGGATCTGGGACAGAATTCACTCTC
ACAATCAGCAGCCTGCAGCCTGAAGATTTTGCAACTTATTACTGTCAACAGCTTA
ATTATTACCCTTTCACTTTCGGCCAGGGGACCAAGGTGGAAATCAAAAGAACTGT
GGCGGCGCCATCTGTCTTCATCTTCCCGCCATCTGATGAGCAGTTGAAATCCGGA
ACTGCCTCTGTTGTGTGCCTGCTGAATAACTTCTATCCCAGAGAGGCCAAAGTAC
AGTGGAAGGTGGATAACGCCCTCCAATCGGGTAACTCCCAGGAGAGTGTCACAG
AGCAGGACAGCAAGGACAGCACCTACAGCCTCAGCAGCACCCTGACGCTGAGCA
AAGCAGACTACGAGAAACACAAAGTCTACGCCTGCGAAGTCACCCATCAGGGCC
TGAGCTCGCCCGTCACAAAGAGCTTCAACAGGGGAGAGTGC
SEQ ID NO: 24; LC C, cDNA
GACATCCAGATGACCCAGTCTCCATCCTTCCTGTCTGCATCTGTGGGAGACAGAG
TCACCATCACTTGCCGGGCCAGTCAGGGCATTAGCAGTTCTTTAGCCTGGTATCA
GCAAAAACCAGGAAAAGCCCCTAAGCTCCTGATCTATACTGCATCCACTTTGCAA
AGTGGGGTCCCATCAAGGTTCAGCGGCAGTGGATCTGGGACAGAATTCACTCTC
ACAATCAGCAGCCTGCAGCCTGAAGATTTTGCAACTTATTACTGTCAACAGCTTA
ATTATTACCCTTTCTCTTTCGGCCAGGGGACCAAGGTGGAAATCAAAAGAACTGT
GGCGGCGCCATCTGTCTTCATCTTCCCGCCATCTGATGAGCAGTTGAAATCCGGA
ACTGCCTCTGTTGTGTGCCTGCTGAATAACTTCTATCCCAGAGAGGCCAAAGTAC
AGTGGAAGGTGGATAACGCCCTCCAATCGGGTAACTCCCAGGAGAGTGTCACAG
AGCAGGACAGCAAGGACAGCACCTACAGCCTCAGCAGCACCCTGACGCTGAGCA
AAGCAGACTACGAGAAACACAAAGTCTACGCCTGCGAAGTCACCCATCAGGGCC
TGAGCTCGCCCGTCACAAAGAGCTTCAACAGGGGAGAGTGC
SEQ ID NO: 25; LCD, cDNA
GACATCCAGATGACCCAGTCTCCATCCTTCCTGTCTGCATCTGTGGGAGACAGAG
TCACCATCACTTGCCGGGCCAGTCAGGGCATTAGCAGTTCTTTAGCCTGGTATCA
GCAAAAACCAGGAAAAGCCCCTAAGCTCCTGATCTATACTGCATCCACTTTGCAA
AGTGGGGTCCCATCAAGGTTCAGCGGCAGTGGATCTGGGACAGAATTCACTCTC
ACAATCAGCAGCCTGCAGCCTGAAGATTTTGCAACTTATTACTGTCAACAGCTTA
ATTATTACCCTTTCACTTTCGGCCAGGGGACCAAGGTGGAAATCAAAAGAACTGT
GGCGGCGCCATCTGTCTTCATCTTCCCGCCATCTGATGAGCAGTTGAAATCCGGA
ACTGCCTCTGTTGTGTGCCTGCTGAATAACTTCTATCCCAGAGAGGCCAAAGTAC
AGTGGAAGGTGGATAACGCCCTCCAATCGGGTAACTCCCAGGAGAGTGTCACAG
AGCAGGACAGCAAGGACAGCACCTACAGCCTCAGCAGCACCCTGACGCTGAGCA
AAGCAGACTACGAGAAACACAAAGTCTACGCCTGCGAAGTCACCCATCAGGGCC
TGAGCTCGCCCGTCACAAAGAGCTTCAACAGGGGAGAGTGC
SEQ ID NO: 26 HC, cDNA
GAGGTGCAGCTGCTCGAGTCGGGGGGAGGCTTGGTACAGCCGGGGGGGTCCCTG
AGACTCTCCTGTGCAGCCTCTGGATTCACCTTTAGTAGCTTTGCCATGAGCTGGG
TCCGCCAGGCTCCAGGGAAGGGGCTGGAGTGGGTCTCAGCAATTAGTGGTAGTG
GTGGTAGCACATACTCCGCAGACTCCGTGAGGGGCCGGTTCACAATCTCCAGAG
ACAATTCCAAGAACACGCTATATCTGCAAATGAACAGCCTGAGAGCCGAGGACA
CGGCCGTATATTACTGTGCCAAAGATCAGGGTGACTGGGACTTTTTATTTGACTA
CTGGGGCCAGGGAACCCTGGTCACCGTCTCCTCAGCCTCCACCAAGGGCCCATCG
GTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAG
GCGCGCTGACCAGCGGCGTGCACACCTTCCCGGCTGTCCTACAGTCCTCAGGACT
CTACTCCCTCAGCAGCGTGGTGACCGTGCCCTCCAGCAGCTTGGGCACCCAGACC
TACATCTGCAACGTGAATCACAAGCCCAGCAACACCAAGGTGGACAAGAGAGTT
GAGCCCAAATCTTGTGACAAAACTCACACATGCCCACCGTGCCCAGCACCTGAA
GCCGCCGGGGGACCGTCAGTCTTCCTCTTCCCCCCAAAACCCAAGGACACCCTCT
ACATCACCCGGGAGCCTGAGGTCACATGCGTGGTGGTGTCCGTGAGCCACGAAG
ACCCTGAGGTCAAGTTCAACTGGTATGTGGACGGCGTGGAGGTGCATAATGCCA
AGACAAAGCCGCGGGAGGAGCAGTACAACAGCACGTACCGTGTGGTCAGCGTCC
TCACCGTCCTGCACCAAGACTGGCTGAATGGCAAGGAGTACAAGTGCAAGGTCT
CCAACAAAGCCCTCCCAGCCCCCATCGAGAAAACCATCTCCAAAGCCAAAGGGC
AGCCCCGAGAACCACAGGTGTACACCCTGCCCCCATCCCGGGAGGAGATGACCA
AGAACCAAGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTC
CCGTGCTGGACTCCGACGGCTCCTTCTTCCTCTATTCCAAGCTCACCGTGGACAA
GAGCAGGTGGCAGCAGGGGAACGTCTTCTCATGCTCCGTGATGCATGAGGCTCT
GCACAACCACTACACGCAGAAGAGCCTCTCCCTGTCTCCGGGCAAA
SEQ ID NO: 27 Recombinant Human Leptin variant
VPIQKVQDDTKTLIKTIVTRINDISHTQSVSSKQKVTGLDFIPGLHPILTLSKMDQTLA
VYQQILTSMPSRNVIQISNDLENLRDLLHVLAFSKSCHLPAASGLETLDSLGGVLEAS
GYSTEVVALSRLQGSLQDMLWQLDLSPGC
SEQ ID NO: 28; human LEPR, full length sequence (NP 002294.2)
MICQKFCVVLLHWEFIYVITAFNLSYPITPWRFKLSCMPPNSTYDYFLLPAGLSKNTS
NSNGHYETAVEPKFNSSGTHFSNLSKTTFHCCFRSEQDRNCSLCADNIEGKTFVSTVN
SLVFQQIDANWNIQCWLKGDLKLFICYVESLFKNLFRNYNYKVHLLYVLPEVLEDSP
LVPQKGSFQMVHCNCSVHECCECLVPVPTAKLNDTLLMCLKITSGGVIFQSPLMSVQ
PINMVKPDPPLGLHMEITDDGNLKISWSSPPLVPFPLQYQVKYSENSTTVIREADKIVS
ATSLLVDSILPGSSYEVQVRGKRLDGPGIWSDWSTPRVFTTQDVIYFPPKILTSVGSN
VSFHCIYKKENKIVPSKEIVWWMNLAEKIPQSQYDVVSDHVSKVTFFNLNETKPRGK
FTYDAVYCCNEHECHHRYAELYVIDVNINISCETDGYLTKMTCRWSTSTIQSLAESTL
QLRYHRSSLYCSDIPSIHPISEPKDCYLQSDGFYECIFQPIFLLSGYTMWIRINHSLGSL
DSPPTCVLPDSVVKPLPPSSVKAEITINIGLLKISWEKPVFPENNLQFQIRYGLSGKEVQ
WKMYEVYDAKSKSVSLPVPDLCAVYAVQVRCKRLDGLGYWSNWSNPAYTVVMDI
KVPMRGPEFWRIINGDTMKKEKNVTLLWKPLMKNDSLCSVQRYVINHHTSCNGTW
SEDVGNHTKFTFLWTEQAHTVTVLAINSIGASVANFNLTFSWPMSKVNIVQSLSAYP
LNSSCVIVSWILSPSDYKLMYFIIEWKNLNEDGEIKWLRISSSVKKYYIHDHFIPIEKYQ
FSLYPIFMEGVGKPKIINSFTQDDIEKHQSDAGLYVIVPVIISSSILLLGTLLISHQRMKK
LFWEDVPNPKNCSWAQGLNFQKPETFEHLFIKHTASVTCGPLLLEPETISEDISVDTS
WKNKDEMMPTTVVSLLSTTDLEKGSVCISDQFNSVNFSEAEGTEVTYEDESQRQPFV
KYATLISNSKPSETGEEQGLINSSVTKCFSSKNSPLKDSFSNSSWEIEAQAFFILSDQHP
NIISPHLTFSEGLDELLKLEGNFPEENNDKKSIYYLGVTSIKKRESGVLLTDKSRVSCPF
PAPCLFTDIRVLQDSCSHFVENNINLGTSSKKTFASYMPQFQTCSTQTHKIMENKMCD
LTV
SEQ ID NO: 29; human LEPR-ECD-His
FNLSYPITPWRFKLSCMPPNSTYDYFLLPAGLSKNTSNSNGHYETAVEPKFNSSGTHF
SNLSKTTFHCCFRSEQDRNCSLCADNIEGKTFVSTVNSLVFQQIDANWNIQCWLKGD
LKLFICYVESLFKNLFRNYNYKVHLLYVLPEVLEDSPLVPQKGSFQMVHCNCSVHEC
CECLVPVPTAKLNDTLLMCLKITSGGVIFQSPLMSVQPINMVKPDPPLGLHMEITDDG
NLKISWSSPPLVPFPLQYQVKYSENSTTVIREADKIVSATSLLVDSILPGSSYEVQVRG
KRLDGPGIWSDWSTPRVFTTQDVIYFPPKILTSVGSNVSFHCIYKKENKIVPSKEIVW
WMNLAEKIPQSQYDVVSDHVSKVTFFNLNETKPRGKFTYDAVYCCNEHECHHRYA
ELYVIDVNINISCETDGYLTKMTCRWSTSTIQSLAESTLQLRYHRSSLYCSDIPSIHPIS
EPKDCYLQSDGFYECIFQPIFLLSGYTMWIRINHSLGSLDSPPTCVLPDSVVKPLPPSSV
KAEITINIGLLKISWEKPVFPENNLQFQIRYGLSGKEVQWKMYEVYDAKSKSVSLPVP
DLCAVYAVQVRCKRLDGLGYWSNWSNPAYTVVMDIKVPMRGPEFWRIINGDTMK
KEKNVTLLWKPLMKNDSLCSVQRYVINHHTSCNGTWSEDVGNHTKFTFLWTEQAH
TVTVLAINSIGASVANFNLTFSWPMSKVNIVQSLSAYPLNSSCVIVSWILSPSDYKLM
YFIIEWKNLNEDGEIKWLRISSSVKKYYIHDHFIPIEKYQFSLYPIFMEGVGKPKIINSFT
QDDIEKHQSDAGAAAHHHHHH
SEQ ID NO: 30; human LEPR ECD-TEV-Fc
FNLSYPITPWRFKLSCMPPNSTYDYFLLPAGLSKNTSNSNGHYETAVEPKFNSSGTHF
SNLSKTTFHCCFRSEQDRNCSLCADNIEGKTFVSTVNSLVFQQIDANWNIQCWLKGD
LKLFICYVESLFKNLFRNYNYKVHLLYVLPEVLEDSPLVPQKGSFQMVHCNCSVHEC
CECLVPVPTAKLNDTLLMCLKITSGGVIFQSPLMSVQPINMVKPDPPLGLHMEITDDG
NLKISWSSPPLVPFPLQYQVKYSENSTTVIREADKIVSATSLLVDSILPGSSYEVQVRG
KRLDGPGIWSDWSTPRVFTTQDVIYFPPKILTSVGSNVSFHCIYKKENKIVPSKEIVW
WMNLAEKIPQSQYDVVSDHVSKVTFFNLNETKPRGKFTYDAVYCCNEHECHHRYA
ELYVIDVNINISCETDGYLTKMTCRWSTSTIQSLAESTLQLRYHRSSLYCSDIPSIHPIS
EPKDCYLQSDGFYECIFQPIFLLSGYTMWIRINHSLGSLDSPPTCVLPDSVVKPLPPSSV
KAEITINIGLLKISWEKPVFPENNLQFQIRYGLSGKEVQWKMYEVYDAKSKSVSLPVP
DLCAVYAVQVRCKRLDGLGYWSNWSNPAYTVVMDIKVPMRGPEFWRIINGDTMK
KEKNVTLLWKPLMKNDSLCSVQRYVINHHTSCNGTWSEDVGNHTKFTFLWTEQAH
TVTVLAINSIGASVANFNLTFSWPMSKVNIVQSLSAYPLNSSCVIVSWILSPSDYKLM
YFIIEWKNLNEDGEIKWLRISSSVKKYYIHDHFIPIEKYQFSLYPIFMEGVGKPKIINSFT
QDDIEKHQSDENLYFQGEPKSSDKTHTCPPCPAPELLGGPSVFLFPPKPKDTLMISRTP
EVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLV
KGFYPSDIA VEWESNGQPENNYKTTPPVLDSDGSFFLYSKLTVDKSRWQQGNVFSCS
VMHEALHNHYTQKSLSLSPGKHHHHHH
SEQ ID NO: 31; cynomolgus monkey LEPR (>XP_005543194.1 (Macaca fascicularis)
MICQKFCVVLLHWEFICVITAFNLSYPITPWRFKLSCMPPNSTYDYFLLPAGLSKNTS
NLNGHYETAVEFNSSDTHFSNLSKTTFHCCFRSEQDRNCSLCADNIEGKTFVSTVNSS
VFQQMGANWNIQCWLKGDLKLFICYVESLFKNPFKNYKHKVHLLYVLPEVLEDSPL
VPQKGSFQMVHCNCSVHERCECLVPVPTAKLNDTLLMCLKITSGGVIFQSPLMSVQP
INMVKPDPPLGLRMEITDDGNLKISWSSPPLVPFPLQYEVKYSENSTTVIREADKIVSA
TSLLVDGILPGSSYEVQVRGKRLDGPGIWSDWSTPHVFTTQDVIYFPPKILTSVGSNV
SFHCIYKNENKIVSSKKIVWWMNLAEKIPQSQYDVVSDHVSKVTFFNLNETKPRGKF
TYDAVYCCNEHECHHRYAELYVIDVNINISCETDGHLTKMTCRWSTNTIQSLAGSTL
QLRYRRSSLYCFDIPSIHPISKPKDCYLQSDGFYECVFQPIFLLSGYTMWIRINHPLGSL
DSPPTCVLPDSVVKPLPPSSVKAEIIKNIGLLKISWEKPVFPENNLQFQIRYGLSGKEIQ
WKMYDVYDAKSKSVSLPVPDFCAVYAVQVRCKRSDGLGLWSNWSNPAYTVVMDI
KVPMRGPEFWRIINGDTMKKEKNVTLLWKPLMKNDSLCSVQRYVINHHTSCNGTW
SEDVGNHTKFTFLWTEQAHTVTVLAINSIGASVANFNLTFSWPMSKVNIVQSLSAYP
LNSSCVILSWILSPSDYKLMYFIIEWKNLNEDGEIKWLRISSSVKKYYIHDHFIPIEKYQ
FSLYPIFMEGVGKPKIINSFTQDNTEKHQNDAGLYVIVPVIISSSILLLGTLLILHQRMK
KLFWEDVPNPKNCSWAQGLNFQKPETFEHLFIKHTASVTCGPLLLEPETISEDISVDTS
WKNKDEMVPTTVVSLLSTTDLEKGSVCISDQFNSVNFSEAEGTEVTCEDESQRQPFV
KYATLISNSKPSETDEEQGLINSSVTKCFSSKNSPLKDSFCNSSWEIEAQAFFILSDQRP
NIILPHLTFSEGLDELLRLEGNFPEENNDEKSIYYLGVTSIKKRESGVLLTDKSRVLCPF
PAPCLFTDIRVLQDSCSHFVENNFNLGTSSKKTFASYMPQFQTCSTQTHKIMENKMC
DLTV
SEQ ID NO: 32; canine LEPR; (>XP_025283785.1 Canis lupus dingo)
MTCQKFCVALLHWEFIYLTTAFNLAYPITPWRFKLSCMPPNTTYDFLLPAGISRNTSN
LNEHYEAVVEAKLNSSSTYISNLSSKTTFHCCFWSKEDKNCSVHADNMEGKAFVST
VNSLVFQQIGANWNIQCWMKEDLKLFICYMESLFKNPFKTYDLKVHLLYVLPEVLE
ESPPVPQKGSFQIVPCNCSVHDSCECHVPVPTAKLNHTLLMYLKITLGGINFQSPLMS
VKPINVVKPDPPLGLHMEITDTGNLKISWSSPTLVPFQLQYQVRYSENSSTNVRKANE
IVSATSLLIDSVLPGSSYEVQVRGKKLDGPGIWGDWSTPLIFITQDVIYFPPKILTSVGS
NVSFHCIYKSENKIVSSKKIVWWLNLAEKIPQSQYNMVGDRVSKVTFPNLNATKPRG
KFTYDAVYCCKEQECHHRYAELYVIDVNINISCETDGYLTKMTCRWSTNAIQSLEGS
TLQLRYHRSSLYCSDVPSIHPISEPKDCHLRRDGFYECIFQPIFLLSGYTMWIKINHSLG
SLDSSPTCVVPDSVVKPLPPSSVKAEITVKIGLLKISWEKPVFPENNLKFQIRYGLNGK
EVQWKMYEVYDAKSKSASVPVPELCAVYAVQVRCKRLDGLGYWSNWSSPAYTVI
MDIKVPTRGPEFWRMIDEDTSRKERNVTLLWKPLMKNDSLCSVRKYVVKHHTSRN
GTWSEDVGNHTKFTFLWTEQAHSVTVLAVNSIGASSVNFNLTFSWPMSKVNTVQSL
SAYPLNSTCVLLSWTLTPSDYYLTYFITEWKILNEDSEIKWLRIPPSVKKYYIHDHFIPI
EKYQFSLYPVFMEGVGKPKTINSFTQDDIEKHQNDAGLYVILLIIISSSILLLGTLLISH
QRMKKLFWEDVPNPKNCSWAQGLNFQKPETFEHLFIKHTESVIFGPLLLEPETISEDIS
VDTSWKNKDEMVPTTMVSLLLTTPDLEKGSICIRDQHNSANFSELESTVVTREDEGR
RQPSVRYATLLTSSKSSEIEEEQGLINSSVSKCFSSKNSLPKGSFSNSSWEIETQAFFILS
DQHPNIILPHLPFSEGLDDLLKLEGNFPEENNGERSVYYLGVTSIKKRESGVFLTDESQ
VLCPFPAHCLFTDIRILQDSCSHLVENNFNLGTSGQKTFVPYMPQFQICSTQTQKIMET
KMCDLTV
SEQ ID NO: 33; rat LEPR; (>XP_032757577.1 leptin receptor [Rattus rattus])
MTCQKFYVVLLHWEFLYVITALNLAYPTSPWRFKLFCAPPSTTDDSFLSPAGGPNNS
SSLKGASEALVEAKFNSSGIYVSELSKTIFHCCFGNEQGQNCSALTGNTEGKTLASVV
KPLVFRQLGVNWDIECWMKGDLTLFICHMEPLLKNPFKNYDSKVHLLYDLPEVIDD
LPLPPLKDSFQTVQCNCSVQECECHVPVPRAKVNYALLMYLEITSAGVSFQSPLMSL
QPMLVVKPDPPLGLRMEVTDDGNLNISWDSQTKAPFPLQYQVKYLENSTIVREAAEI
VSDTSLLVDSVLPGSSYEVQVRSKRLDGSGVWSDWSLPQVFTTQDVMYFPPKILTSV
GSNASFRCIYKNENQTISSKQIVWWMNLAEKIPETQYNTVSDHISKVTFSNLKATRPR
GKFAYDAVYCCNEQACHHRYAELYVIDVNINISCETDGYLTKMTCRWSPSTIQSLVG
STVQMRYHRRSLYCPDNPSIRPTSELKNCVLQRDGFYECVFQPIFLLSGYTMWIRINH
SLGSLDSPPTCVLPDSVVKPLPPSNVKAEITINTGLLKVSWEKPVFPENNLQFQIRYGL
NGKEMQWKTHEVFDAKSKSASLPVSDLCAVYVVQVRCRRLDGLGYWSNWSSPAY
TLVMDVKVPMRGPEFWRIMDGDITKKERNVTLLWKPLMKNDSLCSVRRYVVKHRT
AHNGTWSQDVGNQTNLTFLWAESAHTVTVLAINSIGASLVNFNLTFSWPMSKVNAV
QSLSAYPLSSSCVILSWTLSPDDYSLLYLVIEWKNLNDDDGMKWLRIPSNVNKYYIH
DNFIPIEKYQFSLYPVFMEGVGKPKTINGFTKDDIAKQKNDAGLYVIVPIIISSCVLLLG
TLLISHQRMKKLFWDDVPNPKNCSWAQGLNFQKPETFEHLFTKHAESVIFGPLLLEP
EPISEEISVDTAWKNKDEMVPAAMVSLLLTTPDSTRGSICISDQCNSANFSGAQSTQG
TCEDECQSQPSVKYATLVSNVKPVETDEEQGAIHSSVSQCITRKHSPLRQSFSSSSWEI
EAQAFLLLSDHPPNVISPQLSFSGLDELLELEGNFPEENHGEKSVYYLGVSSVNKREN
DMLLTDEAGVLCPFPAHCLFSDIRILQESCSHFVENNLNLGTSGKNFVPYMPQFQSCS
THSHKIIENKMCDLTV
SEQ ID NO: 34; mouse LEPR; (XP_036019643.1Mus musculus)
MMCQKFYVVLLHWAEFLYVIAALNLAYPISPWKFKLFCGPPNTTDDSFLSPAGAPN
NASALKGASEAIVEAKFNSSGIYVPELSKTVFHCCFGNEQGQNCSALTDNTEGKTLA
SVVKASVFRQLGVNWDIECWMKGDLTLFICHMEPLPKNPFKNYDSKVHLLYDLPEV
IDDSPLPPLKDSFQTVQCNCSLRGCECHVPVPRAKLNYALLMYLEITSAGVSFQSPLM
SLQPMLVVKPDPPLGLHMEVTDDGNLKISWDSQTMAPFPLQYQVKYLENSTIVREA
AEIVSATSLLVDSVLPGSSYEVQVRSKRLDGSGVWSDWSSPQVFTTQDVVYFPPKILT
SVGSNASFHCIYKNENQIISSKQIVWWRNLAEKIPEIQYSIVSDRVSKVTFSNLKATRP
RGKFTYDAVYCCNEQACHHRYAELYVIDVNINISCETDGYLTKMTCRWSPSTIQSLV
GSTVQLRYHRRSLYCPDSPSIHPTSEPKNCVLQRDGFYECVFQPIFLLSGYTMWIRINH
SLGSLDSPPTCVLPDSVVKPLPPSNVKAEITVNTGLLKVSWEKPVFPENNLQFQIRYG
LSGKEIQWKTHEVFDAKSKSASLLVSDLCAVYVVQVRCRRLDGLGYWSNWSSPAY
TLVMDVKVPMRGPEFWRKMDGDVTKKERNVTLLWKPLTKNDSLCSVRRYVVKHR
TAHNGTWSEDVGNRTNLTFLWTEPAHTVTVLAVNSLGASLVNFNLTFSWPMSKVS
AVESLSAYPLSSSCVILSWTLSPDDYSLLYLVIEWKILNEDDGMKWLRIPSNVKKFYI
HDNFIPIEKYQFSLYPVFMEGVGKPKIINGFTKDAIDKQQNDAGLYVIVPIIISSCVLLL
GTLLISHQRMKKLFWDDVPNPKNCSWAQGLNFQKPETFEHLFTKHAESVIFGPLLLE
PEPISEEISVDTAWKNKDEMVPAAMVSLLLTTPDPESSSICISDQCNSANFSGSQSTQV
TCEDECQRQPSVKYATLVSNDKLVETDEEQGFIHSPVSNCISSNHSPLRQSFSSSSWET
EAQTFFLLSDQQPTMISPQLSFSGLDELLELEGSFPEENHREKSVCYLGVTSVNRRESG
VLLTGEAGILCTFPAQCLFSDIRILQERCSHFVENNLSLGTSGENFVPYMPQFQTCSTH
SHKIMENKMCDLTV
SEQ ID NO: 35; rabbit LEPR-ECD-His
FNLAYPVTPWRFKLSCMPANATHDYFLLPAGISKNTSNSSGHYEAIIEDKFNSSDTYF
SNLSQTTFYCCFWSEQDTNCSVRADNIEGKTFVSTVNSLVFQQVGANWDIQCQMKG
DLKLFICYMESLLKNPFKNVGLKVHLLYVLPEMLEDSLLVPQKGTFQMVQCNCSVH
ERCECHVPVPAAKLNYTLLMYFKVTSGGVFLQSPLMSVQLIDAVKPDPPLGLRMEIT
DKGNLKISWSNPAQVPFPLQYQVKYSENSTTIIREVAEIVSATFLLVDSVLPGSSYKA
QVRGRRLDGPGTWSDWSTPQIFVTQDVIYFPPKILTSVGSNVSFHCIYKNEHKIVSSK
QIVWWMNLAEKIPQSQYTVVNDRVSKVTFPNLNATKPRGKFTYDAVYCCRDHECH
HRYAELYVVDVNINISCETDGYLTKMTCRWSANTIQSLVGSTLQLRYHRSSLYCSDIP
SIHPISEPKECHLQRDGFYECIFQPIFLLSGYTMWIRVNHSLGSLDSPPTCVLPDSVVKP
LPPSIVKAEITVNIGLLKLSWEKPVFPENNLQFQIRYGLSGNEKQWKVFEVHDSKSKS
ANLSVPDLCAVYAAQVRCKRLDGLGYWSNWSKPAYTVVKDVKVPVRGPEFWRIID
GDVTKKERNVTLLWKPLMKSDSLCSVSRYVVNHYTSHNGTWSEDVGNHTRFTFIW
AEQVHTVTVVAINSIGASSSNFNLTFSWPVSKVNTVQSLSAYPLNSSCVILSWIPLPSD
YNLMYFIIEWKNLNEDNEIKWLRIPSSVKKYSIHDNFIPIEKYQFSLYPIFMEGVGKPKI
INSFTQDNDEKLQNDAGAAAHHHHHH
SEQ ID NO: 36, Antibody CO HC
QVQLVESGGSVVQPGRSLRLSCAASGFTFSTYAMYWVRQTPGKGLEWVAVLYSDG
SNKYYIDSVKGRFTISRDTSTNTLYLQMSSLRADDSALYYCARLNWDYWYFDLWGR
GTLVTVSSASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVESKYGPPCPP
CPAPEFLGGPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVL
DSDGSFFLYSRLTVDKSRWQEGNVFSCSVMHEALHNHYTQKSLSLSLGK
SEQ ID NO: 37, Antibody CO LC
DIQMTQSPSSLSASVGDRVTITCRASQSISSYLNWYQQKPGKAPKLLIYAASSLQSGV
PSRFSGSGSGTDFTLTISSLQPEDFATYYCQQSYSTPPITFGQGTRLEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO: 38, mouse Fc-Leptin (LEPR Ag Ab surrogate)
VPRDSGCKPCICTVPEVSSVFIFPPKPKDVLTITLTPKVTCVVVDISKDDPEVQFSWFV
DDVEVHTAQTQPREEQFNSTFRSVSELPIMHQDWLNGKEFKCRVNSAAFPAPIEKTIS
KTKGRPKAPQVYTIPPPKEQMAKDKVSLTCMITDFFPEDITVEWQWNGQPAENYKN
TQPIMDTDGSYFVYSKLNVQKSNWEAGNTFTCSVLHEGLHNHHTEKSLSHSPGGGG
SVPIQKVQDDTKTLIKTIVTRINDISHTQSVSSKQKVTGLDFIPGLHPILTLSKMDQTLA
VYQQILTSMPSRNVIQISNDLENLRDLLHVLAFSKSCHLPWASGLETLDSLGGVLEAS
GYSTEVVALSRLQGSLQDMLWQLDLSPGC
SEQ ID NO: 39 amylin receptor agonist
K((γE)₂-CO—(CH₂)₁₈—CO₂H)-[CNTATC]ATQRLADFLRHSSPNFGAIPSSTNVGSRTY-
NH₂
SEQ ID NO: 40 amylin receptor agonist
γE-CNTATCATG-Orn-LAE-αMeF-LVRSSN-NMeN-FGPKLPPTEVGSNTY-NH₂
wherein there is a thioacetal bridge between the cysteines at positions 2 and 7

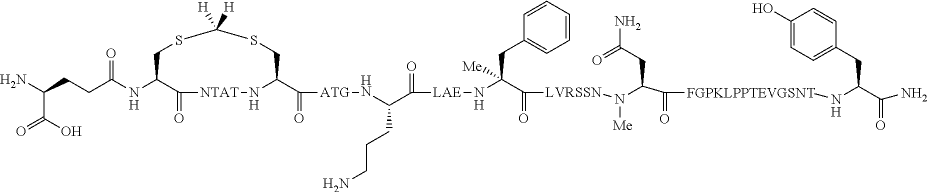

SEQ ID NO: 41 amylin receptor agonist
γE-CNTATCATG-Orn-LAE-αMeF-LVRSSN-NMeN-FGPKLPPTEVGSNTY-NH₂
wherein there is a thioacetal bridge between the cysteines at positions 2 and 7
wherein the lysine at position 26 is attached to a fatty acid linker moiety according to the
formula (γE)₂-CO—(CH₂)₁₈—CO2H

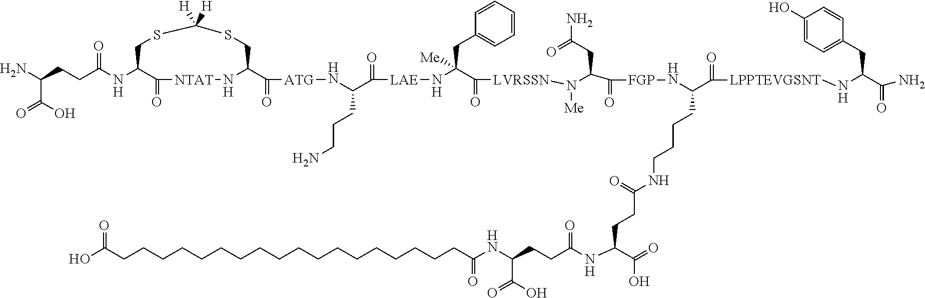

SEQ ID NO: 42 amylin receptor agonist activity
γE-CNTATCATG-Orn-LAE-αMeF-LVRSSN-NMeD-FGPKLPPTEVGSNTY-NH₂
wherein there is a thioacetal bridge between the cysteines at positions 2 and 7

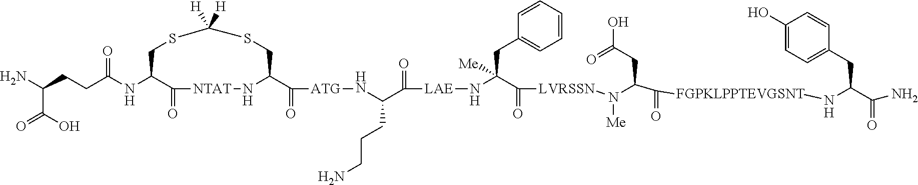

SEQ ID NO: 43 amylin receptor agonist
γE-CNTATCATG-Orn-LAE-αMeF-LVRSSN-NMeD-FGPKLPPTEVGSNTY-NH₂
wherein there is a thioacetal bridge between the cysteines at positions 2 and 7
wherein the lysine at position 26 is attached to a fatty acid linker moiety according to the
formula (γE)₂-CO—(CH₂)₁₈—CO₂H

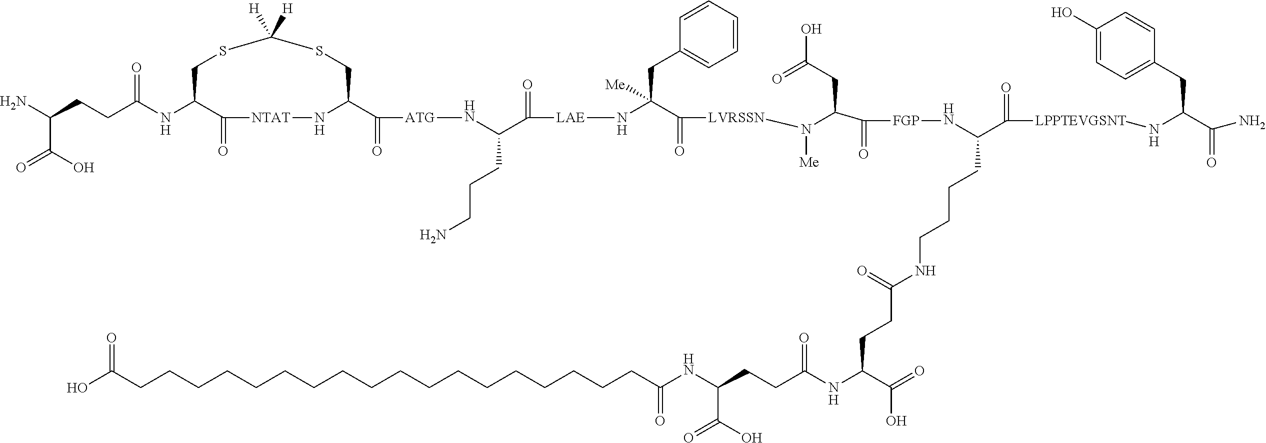

SEQ ID NO: 44 amylin receptor agonist
KCETATCATG-Orn-LAE-αMeF-LVRSSN-NMeD-FGPILPPTEVGSNTY-NH₂
wherein there is a thioacetal bridge between the cysteines at positions 2 and 7

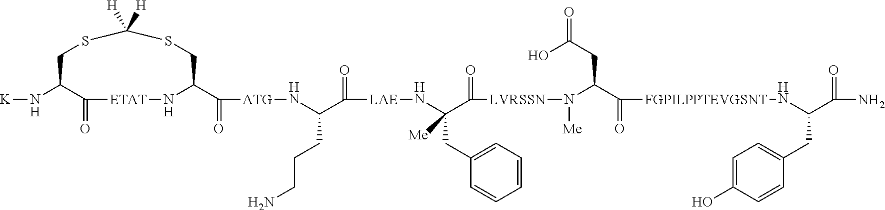

SEQ ID NO: 45 amylin receptor agonist activity
KCETATCATG-Orn-LAE-αMeF-LVRSSN-NMeD-FGPILPPTEVGSNTY-NH₂
wherein there is a thioacetal bridge between the cysteines at positions 2 and 7
wherein the lysine at position 1 is attached to a fatty acid linker moiety according to the
formula (γE)₂-CO—(CH₂)₁₈—CO₂H

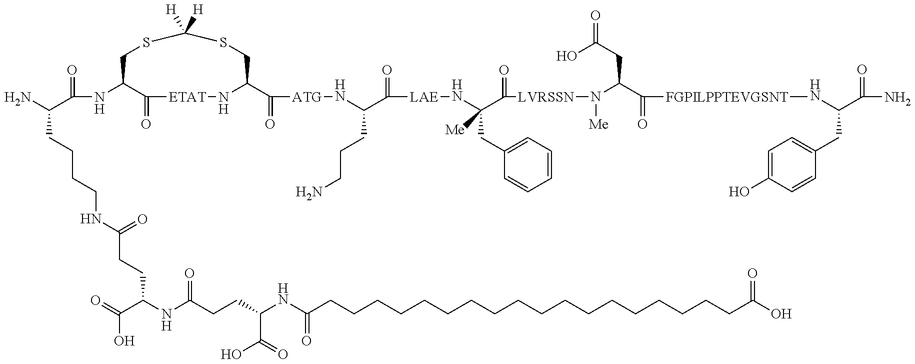

SEQ ID NO: 46 amylin receptor agonist
KCETATCATG-Orn-αMeL-AEFLVRSSHNFGPILPPTEVGSNTY-NH₂
wherein there is a thioacetal bridge between the cysteines at positions 2 and 7

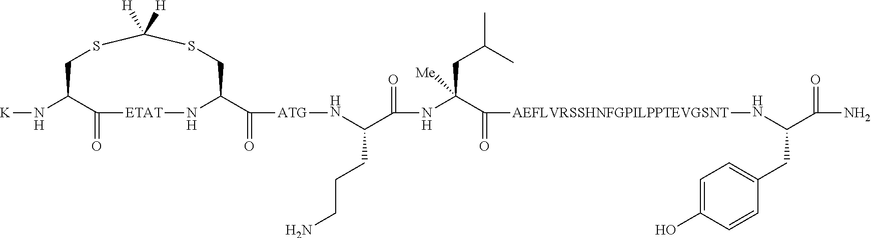

SEQ ID NO:47 amylin receptor agonist
KCETATCATG-Orn-αMeL-AEFLVRSSHNFGPILPPTEVGSNTY-NH₂
wherein there is a thioacetal bridge between the cysteines at positions 2 and 7
wherein the lysine at position 1 is attached to a fatty acid linker moiety according to the
formula (γE)₂-CO—(CH₂)₁₈—CO₂H

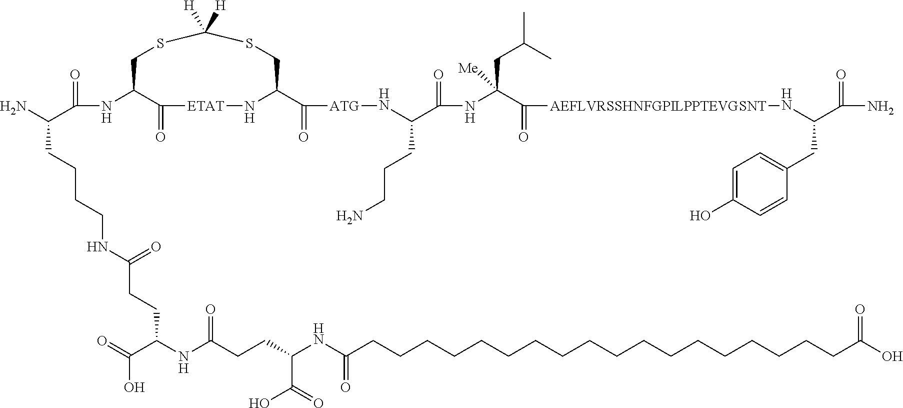

SEQ ID NO:48 dual amylin and calcitonin receptor agonist
Acetyl-ASHLSTAVLGKLS-Aib-ELHKLEDYPRTDVGAESP-NH₂
SEQ ID NO:49 dual amylin and calcitonin receptor agonist
Acetyl-ASHLSTAVLGK((2-[2-(2-Amino-ethoxy)-ethoxy]-acetyl)₂-(γ-Glu)-
CO-(CH₂)₁₈—CO₂H)LS-Aib-ELHKLEDYPRTDVGAESP-NH₂
SEQ ID NO: 50 amylin receptor agonist
KCNTATCATQRLANFLVHSSNNFGPILPPTNVGSNTY-NH₂
wherein there is a disulfide bond at positions 2 and 7
SEQ ID NO: 51 amylin receptor agonist
CO₂H—(CH₂)₁₈—CO-γE-KCNTATCATQRLAEFLRHSSNNFGPILPPTNVGSNTP-NH₂
sequence modifications relative to h-amylin: 14E, 17R, 25P, 28P, 29P, 37P
SEQ ID NO: 52 GLP-1 receptor agonist
H-Aib-EGTFTSDVSSYLEGQAAKEFIAWLVRGRG
wherein K at position 20 is chemically modified by conjugation of the epsilon-amino group
of the K side chain with (2-[2-(2-Amino-ethoxy)-ethoxy]-acetyl)₂-(γE)-CO-(CH₂)₁₆—CO₂H.

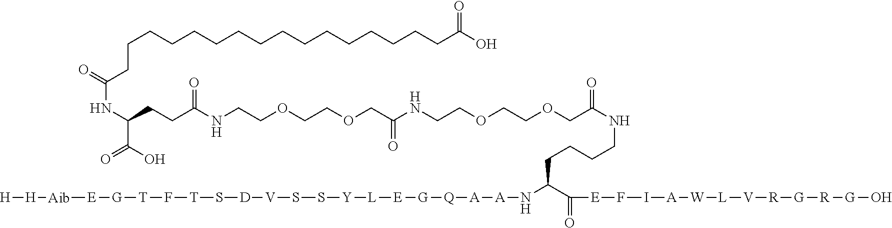

SEQ ID NO: 53, dual agonist of the GLP-1 and GIP receptors
Y-Aib-EGTFTSDYSI-Aib-LDKIAQK((2-[2-(2-Amino-ethoxy)-ethoxy]-acetyl)₂-
γE2-CO—(CH₂)₁₈—CO₂H)A-1Nal-VQWLIAGGPSSGAPPPS-NH₂
SEQ ID NO:54 dual agonist of GLP-1 receptor and GIP receptor
Y-Aib-EGTFTSDYSI-Aib-LDKIAQK((2-[2-(2-Amino-ethoxy)-ethoxy]-acetyl)₂-(γE)-
CO—(CH₂)₁₈—CO₂H)AFVQWLIAGGPSSGAPPPS-NH₂
wherein K at position 20 is chemically modified through conjugation to the epsilon-amino
group of the K side-chain with (2-[2-(2-Amino-ethoxy)-ethoxy]-acetyl)₂-(γE)1-
CO—(CH₂)₁₈—CO₂H; and the C-terminal amino acid is amidated as a C-terminal primary amide

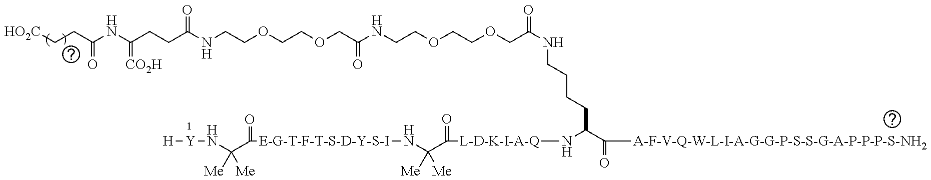

L-Tyrosy1-2-methylalanyl-L-α-glutamylglycyl-L-threonyl-L-phenylalanyl-L-threonyl-L-
sery1-L-α-asparty1-L-tyrosyl-L-seryl-L-isoleucyl-2-methylalanyl-L-leucyl-L-α-aspartyl-L-
lysyl-L-isoleucyl-L-alany1-L-glutaminyl-N⁶-[(22S)-22,42-dicarboxy-1,10,19,24-tetraoxo-
3,6,12,15-tetraoxa-9,18,23-triazadotetracont-1-yl]-L-lysyl-L-alanyl-L-phenylalanyl-L-valyl-
L-glutaminyl-L-tryptophyl-L-leucyl-L-isoleucyl-L-alanylglycylglycyl-L-prolyl-L-sery1-L-
serylglycyl-L-alanyl-L-prolyl-L-prolyl-L-prolyl-L-serinamide
SEQ ID NO: 55, triagonist of GIP/GLP-1/glucagon receptors
Y-Aib-QGTFTSDYSILLDKK((2-[2-(2-Amino-ethoxy)-ethoxy]-acetyl)₂-(γE)-CO—(CH₂)₁₈-
CO₂H)AQ-Aib-AFIEYLLEGGPSSGAPPPS-NH₂
SEQ ID NO: 56 tri-agonist GLP1R/GIPR/GCGR
Y-Aib-QGTFTSDYSI-αMeL-LDKK((2-[2-(2-Amino-ethoxy)-ethoxy]-acetyl)-(
γE)-CO—-(CH₂)₁₈—CO₂H)AQ-Aib-AFIEYLLEGGPSSGAPPPS-NH₂
wherein K at position 17 is chemically modified by conjugation of the epsilon-amino group
of the K side chain with (2-[2-(2-Amino-ethoxy)-ethoxy]-acetyl)-(γE)-CO-(CH₂)₁₈—CO2H;
and the C-terminal amino acid is amidated as a C-terminal primary amide.

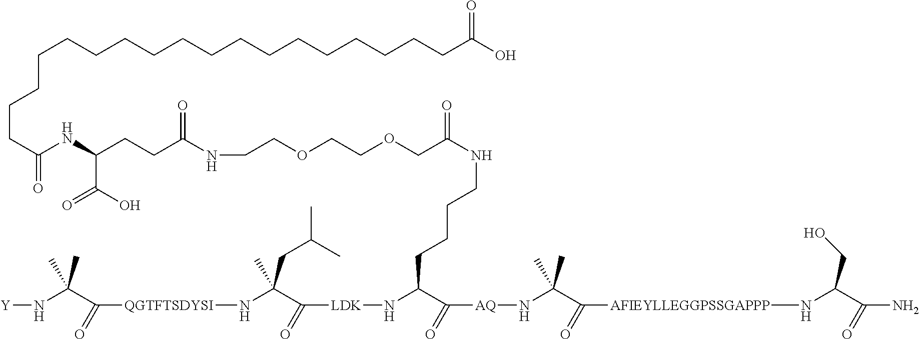

SEQ ID NO: 57; glucagon receptor agonist
Y-Aib-QGTFTSDYSKYLD-Aib-KKAK((2-[2-(2-Amino-ethoxy)-ethoxy]-acetyl)₂-(γE)₂-
CO—(CH₂)₁₆—CO₂H)EFVEWLLETGPSSGAPPPS-NH₂
SEQ ID NO: 58; CYNO LEPR-ECD-His
FNLSYPITPWRFKLSCMPPNSTYDYFLLPAGLSKNTSNLNGHYETAVEFNSSDTHFSN
LSKTTFHCCFRSEQDRNCSLCADNIEGKTFVSTVNSSVFQQMGANWNIQCWLKGDL
KLFICYVESLFKNPFKNYKHKVHLLYVLPEVLEDSPLVPQKGSFQMVHCNCSVHERC
ECLVPVPTAKLNDTLLMCLKITSGGVIFQSPLMSVQPINMVKPDPPLGLRMEITDDGN
LKISWSSPPLVPFPLQYEVKYSENSTTVIREADKIVSATSLLVDGILPGSSYEVQVRGK
RLDGPGIWSDWSTPHVFTTQDVIYFPPKILTSVGSNVSFHCIYKNENKIVSSKKIVWW
MNLAEKIPQSQYDVVSDHVSKVTFFNLNETKPRGKFTYDAVYCCNEHECHHRYAEL
YVIDVNINISCETDGHLTKMTCRWSTNTIQSLAGSTLQLRYRRSSLYCFDIPSIHPISKP
KDCYLQSDGFYECVFQPIFLLSGYTMWIRINHPLGSLDSPPTCVLPDSVVKPLPPSSVK
AEIIKNIGLLKISWEKPVFPENNLQFQIRYGLSGKEIQWKMYDVYDAKSKSVSLPVPD
FCAVYAVQVRCKRSDGLGLWSNWSNPAYTVVMDIKVPMRGPEFWRIINGDTMKKE
KNVTLLWKPLMKNDSLCSVQRYVINHHTSCNGTWSEDVGNHTKFTFLWTEQAHTV
TVLAINSIGASVANFNLTFSWPMSKVNIVQSLSAYPLNSSCVILSWILSPSDYKLMYFII
EWKNLNEDGEIKWLRISSSVKKYYIHDHFIPIEKYQFSLYPIFMEGVGKPKIINSFTQD
NTEKHQNDGHHHHHH
SEQ ID NO: 59; canine LEPR-ECD-His
FNLAYPITPWRFKLSCMPPNTTYDFLLPAGISRNTSNLNEHYEAVVEAKLNSSSTYIS
NLSSKTTFHCCFWSKEDKNCSVHADNMEGKAFVSTVSSLVFQQIGANWNIQCWMK
EDLKLFICYMESLFKNPFKTYDLKVHLLYVLPEVLEESPPVPQKGGFQIVPCNCSVHD
SCECHVPVPTAELNHTLLMYLKITLGGINFQSPLMSVKPINVVKPDPPLGLHMEITDT
GNLKISWSSPTLVPFQLQYQVRYSENSSTNVRKANEIVSATSLLIDSVLPGSSYEVQV
RGKKLDGPGIWGDWSTPLIFITQDVIYFPPKILTSVGSNVSFHCIYKSENKIVSSKKIV
WWLNLAEKIPQSQYNMVGDRVSKVTFPNLNATKPRGKFTYDAVYCCKEQECHHRY
AELYVIDVNINISCETDGYLTKMTCRWSTNAIQSLEGSTLQLRYHRSSLYCSDVPSIHP
ISEPKDCHLRRDGFYECIFQPIFLLSGYTMWIKINHSLGSLDSSPTCVVPDSVVKPLPPS
SVKAEITVKIGLLKISWEKPVFPENNLKFQIRYGLNGKEVQWKIYEVYDTKLKSTSLP
VPDLCAVYAVQVRCKRLDGLGYWSNWSTPAYTVVTDVKVPTRGPEFWRMIDEDTS
RKERNVTLLWKPLMKNDSLCSVRKYVVKHHTSRNGTWSEDVGNHTKFTFLWTEQA
HSVTVLAVNSIGASSVNFNLTFSWPMSKVNTVQSLSAYPLNSTCVLLSWTLTPSDYY
LTYFITEWKILNEDSEIKWLRIPPSVKKYYIHDHFIPIEKYQFSLYPVFMEGVGKPKTIN
SFTQDDIEKHQNDHHHHHH
SEQ ID NO: 60; Rat LEPR-ECD-His
LNLAYPTSPWRFKLFCAPPSTTDDSFLSPAGVPNNTSSLKGASEALVEAKFNSTGIYV
SELSKTIFHCCFGNEQGQNCSALTGNTEGKTLASVVKPLVFRQLGVNWDIECWMKG
DLTLFICHMEPLLKNPFKNYDSKVHLLYDLPEVIDDLPLPPLKDSFQTVQCNCSVREC
ECHVPVPRAKVNYALLMYLEITSAGVSFQSPLMSLQPMLVVKPDPPLGLRMEVTDD
GNLKISWDSQTKAPFPLQYQVKYLENSTIVREAAEIVSDTSLLVDSVLPGSSYEVQVR
SKRLDGSGVWSDWSLPQLFTTQDVMYFPPKILTSVGSNASFCCIYKNENQTISSKQIV
WWMNLAEKIPETQYNTVSDHISKVTFSNLKATRPRGKFTYDAVYCCNEQACHHRY
AELYVIDVNINISCETDGYLTKMTCRWSPSTIQSLVGSTVQLRYHRRSLYCPDNPSIRP
TSELKNCVLQTDGFYECVFQPIFLLSGYTMWIRINHSLGSLDSPPTCVLPDSVVKPLPP
SNVKAEITINTGLLKVSWEKPVFPENNLQFQIRYGLNGKEIQWKTHEVFDAKSKSAS
LPVSDLCAVYVVQVRCRRLDGLGYWSNWSSPAYTLVMDVKVPMRGPEFWRIMDG
DITKKERNVTLLWKPLMKNDSLCSVRRYVVKHRTAHNGTWSQDVGNQTNLTFLW
AESAHTVTVLAINSIGASLVNFNLTFSWPMSKVNAVQSLSAYPLSSSCVILSWTLSPN
DYSLLYLVIEWKNLNDDDGMKWLRIPSNVNKYYIHDNFIPIEKYQFSLYPVFMEGVG
KPKIINGFTKDDIAKQQNDAGAAAHHHHHH
SEQ ID NO:61; Mouse LEPR-ECD-His
LNLAYPISPWKFKLFCGPPNTTDDSFLSPAGAPNNASALKGASEAIVEAKFNSSGIYV
PELSKTVFHCCFGNEQGQNCSALTDNTEGKTLASVVKASVFRQLGVNWDIECWMK
GDLTLFICHMEPLPKNPFKNYDSKVHLLYDLPEVIDDSPLPPLKDSFQTVQCNCSLRG
CECHVPVPRAKLNYALLMYLEITSAGVSFQSPLMSLQPMLVVKPDPPLGLHMEVTD
DGNLKISWDSQTMAPFPLQYQVKYLENSTIVREAAEIVSATSLLVDSVLPGSSYEVQ
VRSKRLDGSGVWSDWSSPQVFTTQDVVYFPPKILTSVGSNASFHCIYKNENQIISSKQ
IVWWRNLAEKIPEIQYSIVSDRVSKVTFSNLKATRPRGKFTYDAVYCCNEQACHHRY
AELYVIDVNINISCETDGYLTKMTCRWSPSTIQSLVGSTVQLRYHRRSLYCPDSPSIHP
TSEPKNCVLQRDGFYECVFQPIFLLSGYTMWIRINHSLGSLDSPPTCVLPDSVVKPLPP
SNVKAEITVNTGLLKVSWEKPVFPENNLQFQIRYGLSGKEIQWKTHEVFDAKSKSAS
LLVSDLCAVYVVQVRCRRLDGLGYWSNWSSPAYTLVMDVKVPMRGPEFWRKMDG
DVTKKERNVTLLWKPLTKNDSLCSVRRYVVKHRTAHNGTWSEDVGNRTNLTFLWT
EPAHTVTVLAVNSLGASLVNFNLTFSWPMSKVSAVESLSAYPLSSSCVILSWTLSPDD
YSLLYLVIEWKILNEDDGMKWLRIPSNVKKFYIHDNFIPIEKYQFSLYPVFMEGVGKP
KIINGFTKDAIDKQQNDAGAAAHHHHHHSGS

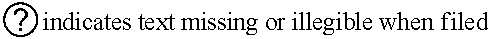

We claim:

1. An antibody that binds leptin receptor (LEPR), wherein the antibody comprises a heavy chain variable region (VH) and a light chain variable region (VL), wherein the VH comprises heavy chain complementarity determining regions (HCDR) HCDR1, HCDR2, and HCDR3, and the VL comprises light chain complementarity determining regions (LCDR) LCDR1, LCDR2, and LCDR3, wherein
a) the HCDR1 comprises SEQ ID NO:7,
b) the HCDR2 comprises SEQ ID NO:8,
c) the HCDR3 comprises SEQ ID NO:9,
d) the LCDR1 comprises SEQ ID NO:1 or 2,
e) the LCDR2 comprises SEQ ID NO:3, and
f) the LCDR3 comprises SEQ ID NO:4, 5, or 6.

2. The antibody of claim 1, wherein the LCDR1 comprises SEQ ID NO: 1.

3. The antibody of claim 1, wherein the LCDR1 comprises SEQ ID NO: 2.

4. The antibody of claim 1, wherein the antibody comprises a human IgG1 or IgG4 subclass.

5. The antibody of claim 1, wherein the antibody comprises one of: (a) the VH comprises SEQ ID NO:18 and the VL comprises SEQ ID NO:16; (b) the VH comprises SEQ ID NO:18 and the VL comprises SEQ ID NO:10; (c) the VH comprises SEQ ID NO:18 and the VL comprises SEQ ID NO:12; and (d) the VH comprises SEQ ID NO:18 and the VL comprises SEQ ID NO:14.

6. The antibody of claim 1, wherein the antibody comprises one of: (a) a heavy chain (HC) comprising SEQ ID NO:19, and a light chain (LC) comprising SEQ ID NO:17;
(b) a heavy chain (HC) comprising SEQ ID NO:19, and a light chain (LC) comprising SEQ ID NO:11; (c) a heavy chain (HC) comprising SEQ ID NO:19, and (d) a light chain (LC) comprising SEQ ID NO:13; and a heavy chain (HC) comprising SEQ ID NO: 19, and a light chain (LC) comprising SEQ ID NO:15.

7. The antibody of claim 1, wherein the antibody comprises at least one of: binds human LEPR with a dissociation equilibrium constant (K_{D}) of between 1E-10 M and 3.0E-8 M, binds cynomolgus LEPR with a dissociation equilibrium constant (K_{D}) of between 1E-10 M and 5.0E-7 M, or binds canine LEPR with a dissociation equilibrium constant (K_{D}) of between 1E-10 M and 5.0E-6 M; wherein the K_{D} is determined by surface plasmon resonance (SPR) assay.

8. The antibody of claim 1, wherein the antibody comprises at least one of: EC₅₀ (human) of between 0.40 nM and 0.01 nM, EC₅₀ (cynomolgus) of between 2.0 nM and 0.01 nM, and EC₅₀ (canine) of between 1.0 nM and 0.01 nM; wherein the EC₅₀ is determined by pSTAT3 assay.

9. The antibody of claim 1, wherein the antibody provides at least one of: between 55% and 65% agonism of human LEPR, as compared to human leptin, between 20% and 30% agonism of cynomolgus LEPR, as compared to human leptin, and between 65% and 85% agonism of canine LEPR, as compared to human leptin; wherein agonism is determined by pSTAT3 assay.

10. A nucleic acid comprising a sequence encoding at least one of SEQ ID NOs: 1-20.

11. A cell comprising the nucleic acid of claim 10.

12. A pharmaceutical composition comprising the antibody of claim 1, and a pharmaceutically acceptable excipient, diluent, or carrier.

13. A pharmaceutical composition comprising the antibody of claim 1, and a second therapeutic agent.

14. The pharmaceutical composition of claim 12, wherein the second therapeutic agent comprises an agent as depicted in any one of SEQ ID NO:40-47.

15. The pharmaceutical composition of claim 12, wherein the second therapeutic agent comprises an agent depicted in any one of SEQ ID NO: 50-51.

16. The pharmaceutical composition of claim 12, wherein the second therapeutic agent comprises GLP-1 agonist activity or is a dual agonist of the GLP-1 and GIP receptors.

17. The pharmaceutical composition of claim 16, wherein the second therapeutic agent comprises SEQ ID NO: 54 or SEQ ID NO:56.

18. A method of treating obesity, said method comprising simultaneous, separate, or sequential administration of an antibody of claim 1 and a second therapeutic agent comprising a sequence selected from SEQ ID NOs: 39-57.

19. A method of treating obesity in a subject in need thereof, said method comprising administering to the subject a therapeutically effective amount of the antibody or composition of claim 1.

20. A method of treating a lipodystrophy disorder in a subject in need thereof, said method comprising administering to the subject a therapeutically effective amount of the antibody of claim 1.


SEQUENCE LISTING (61 sequences)

SEQ ID NO: 1 - protein, 11 aa, synthetic construct
RASQGISSSLA

SEQ ID NO: 2 - protein, 11 aa, synthetic construct
RVSQGISSSLA

SEQ ID NO: 3 - protein, 8 aa, synthetic construct
YTASTLQS

SEQ ID NO: 4 - protein, 9 aa, synthetic construct
QQLIYYPFT

SEQ ID NO: 5 - protein, 9 aa, synthetic construct
QQLNYYPFT

SEQ ID NO: 6 - protein, 9 aa, synthetic construct
QQLNYYPFS

SEQ ID NO: 7 - protein, 13 aa, synthetic construct
AASGFTFSSFAMS

SEQ ID NO: 8 - protein, 10 aa, synthetic construct
AISGSGGSTY

SEQ ID NO: 9 - protein, 13 aa, synthetic construct
AKDQGDWDFLFDY

SEQ ID NO: 10 - protein, 107 aa, synthetic construct
DIQMTQSPSFLSASVGDRVTITCRASQGISSSLAWYQQKPGKAPKLLIYTASTLQSGVPSRFSGSGSGTEFTLTISSLQPEDFATYYCQQLIYYPFTFGQGTKVEIK

SEQ ID NO: 11 - protein, 214 aa, synthetic construct
DIQMTQSPSFLSASVGDRVTITCRASQGISSSLAWYQQKPGKAPKLLIYTASTLQSGVPSRFSGSGSGTEFTLTISSLQPEDFATYYCQQLIYYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 107 aa, synthetic construct
DIQMTQSPSFLSASVGDRVTITCRVSQGISSSLAWYQQKPGKAPKLLIYTASTLQSGVPSRFSGSGSGTEFTLTISSLQPEDFATYYCQQLNYYPFTFGQGTKVEIK

SEQ ID NO: 13 - protein, 214 aa, synthetic construct
DIQMTQSPSFLSASVGDRVTITCRVSQGISSSLAWYQQKPGKAPKLLIYTASTLQSGVPSRFSGSGSGTEFTLTISSLQPEDFATYYCQQLNYYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 107 aa, synthetic construct
DIQMTQSPSFLSASVGDRVTITCRASQGISSSLAWYQQKPGKAPKLLIYTASTLQSGVPSRFSGSGSGTEFTLTISSLQPEDFATYYCQQLNYYPFSFGQGTKVEIK

SEQ ID NO: 15 - protein, 214 aa, synthetic construct
DIQMTQSPSFLSASVGDRVTITCRASQGISSSLAWYQQKPGKAPKLLIYTASTLQSGVPSRFSGSGSGTEFTLTISSLQPEDFATYYCQQLNYYPFSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 107 aa, synthetic construct
DIQMTQSPSFLSASVGDRVTITCRASQGISSSLAWYQQKPGKAPKLLIYTASTLQSGVPSRFSGSGSGTEFTLTISSLQPEDFATYYCQQLNYYPFTFGQGTKVEIK

SEQ ID NO: 17 - protein, 214 aa, synthetic construct
DIQMTQSPSFLSASVGDRVTITCRASQGISSSLAWYQQKPGKAPKLLIYTASTLQSGVPSRFSGSGSGTEFTLTISSLQPEDFATYYCQQLNYYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 120 aa, synthetic construct
EVQLLESGGGLVQPGGSLRLSCAASGFTFSSFAMSWVRQAPGKGLEWVSAISGSGGSTYSADSVRGRFTISRDNSKNTLYLQMNSLRAEDTAVYYCAKDQGDWDFLFDYWGQGTLVTVSS

SEQ ID NO: 19 - protein, 450 aa, synthetic construct
EVQLLESGGGLVQPGGSLRLSCAASGFTFSSFAMSWVRQAPGKGLEWVSAISGSGGSTYSADSVRGRFTISRDNSKNTLYLQMNSLRAEDTAVYYCAKDQGDWDFLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 20 aa, synthetic construct
METDTLLLWVLLLWVPGSTG

SEQ ID NO: 21 - DNA, 60 bp, synthetic construct
  source 1..60: mol_type = other DNA
atggagacagacacactcctgctatgggtactgctgctctgggttccaggatctaccggt

SEQ ID NO: 22 - DNA, 642 bp, synthetic construct
  source 1..642: mol_type = other DNA
gacatccagatgacccagtctccatccttcctgtctgcatctgtgggagacagagtcaccatcacttgccgggccagtcagggcattagcagttctttagcctggtatcagcaaaaaccaggaaaagcccctaagctcctgatctatactgcatccactttgcaaagtggggtcccatcaaggttcagcggcagtggatctgggacagaattcactctcacaatcagcagcctgcagcctgaagattttgcaacttattactgtcaacagcttatttattaccctttcactttcggccaggggaccaaggtggaaatcaaaagaactgtggcggcgccatctgtcttcatcttcccgccatctgatgagcagttgaaatccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 23 - DNA, 642 bp, synthetic construct
  source 1..642: mol_type = other DNA
gacatccagatgacccagtctccatccttcctgtctgcatctgtgggagacagagtcaccatcacttgccgggtcagtcagggcattagcagttctttagcctggtatcagcaaaaaccaggaaaagcccctaagctcctgatctatactgcatccactttgcaaagtggggtcccatcaaggttcagcggcagtggatctgggacagaattcactctcacaatcagcagcctgcagcctgaagattttgcaacttattactgtcaacagcttaattattaccctttcactttcggccaggggaccaaggtggaaatcaaaagaactgtggcggcgccatctgtcttcatcttcccgccatctgatgagcagttgaaatccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 24 - DNA, 642 bp, synthetic construct
  source 1..642: mol_type = other DNA
gacatccagatgacccagtctccatccttcctgtctgcatctgtgggagacagagtcaccatcacttgccgggccagtcagggcattagcagttctttagcctggtatcagcaaaaaccaggaaaagcccctaagctcctgatctatactgcatccactttgcaaagtggggtcccatcaaggttcagcggcagtggatctgggacagaattcactctcacaatcagcagcctgcagcctgaagattttgcaacttattactgtcaacagcttaattattaccctttctctttcggccaggggaccaaggtggaaatcaaaagaactgtggcggcgccatctgtcttcatcttcccgccatctgatgagcagttgaaatccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 25 - DNA, 642 bp, synthetic construct
  source 1..642: mol_type = other DNA
gacatccagatgacccagtctccatccttcctgtctgcatctgtgggagacagagtcaccatcacttgccgggccagtcagggcattagcagttctttagcctggtatcagcaaaaaccaggaaaagcccctaagctcctgatctatactgcatccactttgcaaagtggggtcccatcaaggttcagcggcagtggatctgggacagaattcactctcacaatcagcagcctgcagcctgaagattttgcaacttattactgtcaacagcttaattattaccctttcactttcggccaggggaccaaggtggaaatcaaaagaactgtggcggcgccatctgtcttcatcttcccgccatctgatgagcagttgaaatccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 26 - DNA, 1350 bp, synthetic construct
  source 1..1350: mol_type = other DNA
gaggtgcagctgctcgagtcggggggaggcttggtacagccgggggggtccctgagactctcctgtgcagcctctggattcacctttagtagctttgccatgagctgggtccgccaggctccagggaaggggctggagtgggtctcagcaattagtggtagtggtggtagcacatactccgcagactccgtgaggggccggttcacaatctccagagacaattccaagaacacgctatatctgcaaatgaacagcctgagagccgaggacacggccgtatattactgtgccaaagatcagggtgactgggactttttatttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctctacatcacccgggagcctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 27 - protein, 146 aa, synthetic construct
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPAASGLETLDSLGGVLEASGYSTEVVALSRLQGSLQDMLWQLDLSPGC

SEQ ID NO: 28 - protein, 1165 aa, Homo sapiens
MICQKFCVVLLHWEFIYVITAFNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDAGLYVIVPVIISSSILLLGTLLISHQRMKKLFWEDVPNPKNCSWAQGLNFQKPETFEHLFIKHTASVTCGPLLLEPETISEDISVDTSWKNKDEMMPTTVVSLLSTTDLEKGSVCISDQFNSVNFSEAEGTEVTYEDESQRQPFVKYATLISNSKPSETGEEQGLINSSVTKCFSSKNSPLKDSFSNSSWEIEAQAFFILSDQHPNIISPHLTFSEGLDELLKLEGNFPEENNDKKSIYYLGVTSIKKRESGVLLTDKSRVSCPFPAPCLFTDIRVLQDSCSHFVENNINLGTSSKKTFASYMPQFQTCSTQTHKIMENKMCDLTV

SEQ ID NO: 29 - protein, 829 aa, Homo sapiens
FNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDAGAAAHHHHHH

SEQ ID NO: 30 - protein, 1063 aa, Homo sapiens
FNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDENLYFQGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 31 - protein, 1163 aa, Macaca fascicularis
MICQKFCVVLLHWEFICVITAFNLSYPITPWRFKLSCMPPNSTYDYFLLPAGLSKNTSNLNGHYETAVEFNSSDTHFSNLSKTTFHCCFRSEQDRNCSLCADNIEGKTFVSTVNSSVFQQMGANWNIQCWLKGDLKLFICYVESLFKNPFKNYKHKVHLLYVLPEVLEDSPLVPQKGSFQMVHCNCSVHERCECLVPVPTAKLNDTLLMCLKITSGGVIFQSPLMSVQPINMVKPDPPLGLRMEITDDGNLKISWSSPPLVPFPLQYEVKYSENSTTVIREADKIVSATSLLVDGILPGSSYEVQVRGKRLDGPGIWSDWSTPHVFTTQDVIYFPPKILTSVGSNVSFHCIYKNENKIVSSKKIVWWMNLAEKIPQSQYDVVSDHVSKVTFFNLNETKPRGKFTYDAVYCCNEHECHHRYAELYVIDVNINISCETDGHLTKMTCRWSTNTIQSLAGSTLQLRYRRSSLYCFDIPSIHPISKPKDCYLQSDGFYECVFQPIFLLSGYTMWIRINHPLGSLDSPPTCVLPDSVVKPLPPSSVKAEIIKNIGLLKISWEKPVFPENNLQFQIRYGLSGKEIQWKMYDVYDAKSKSVSLPVPDFCAVYAVQVRCKRSDGLGLWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVILSWILSPSDYKLMYFIIEWKNLNEDGEIKWLRISSSVKKYYIHDHFIPIEKYQFSLYPIFMEGVGKPKIINSFTQDNTEKHQNDAGLYVIVPVIISSSILLLGTLLILHQRMKKLFWEDVPNPKNCSWAQGLNFQKPETFEHLFIKHTASVTCGPLLLEPETISEDISVDTSWKNKDEMVPTTVVSLLSTTDLEKGSVCISDQFNSVNFSEAEGTEVTCEDESQRQPFVKYATLISNSKPSETDEEQGLINSSVTKCFSSKNSPLKDSFCNSSWEIEAQAFFILSDQRPNIILPHLTFSEGLDELLRLEGNFPEENNDEKSIYYLGVTSIKKRESGVLLTDKSRVLCPFPAPCLFTDIRVLQDSCSHFVENNFNLGTSSKKTFASYMPQFQTCSTQTHKIMENKMCDLTV

SEQ ID NO: 32 - protein, 1166 aa, Canis lupus
  source 1..1166: note = dingo
MTCQKFCVALLHWEFIYLTTAFNLAYPITPWRFKLSCMPPNTTYDFLLPAGISRNTSNLNEHYEAVVEAKLNSSSTYISNLSSKTTFHCCFWSKEDKNCSVHADNMEGKAFVSTVNSLVFQQIGANWNIQCWMKEDLKLFICYMESLFKNPFKTYDLKVHLLYVLPEVLEESPPVPQKGSFQIVPCNCSVHDSCECHVPVPTAKLNHTLLMYLKITLGGINFQSPLMSVKPINVVKPDPPLGLHMEITDTGNLKISWSSPTLVPFQLQYQVRYSENSSTNVRKANEIVSATSLLIDSVLPGSSYEVQVRGKKLDGPGIWGDWSTPLIFITQDVIYFPPKILTSVGSNVSFHCIYKSENKIVSSKKIVWWLNLAEKIPQSQYNMVGDRVSKVTFPNLNATKPRGKFTYDAVYCCKEQECHHRYAELYVIDVNINISCETDGYLTKMTCRWSTNAIQSLEGSTLQLRYHRSSLYCSDVPSIHPISEPKDCHLRRDGFYECIFQPIFLLSGYTMWIKINHSLGSLDSSPTCVVPDSVVKPLPPSSVKAEITVKIGLLKISWEKPVFPENNLKFQIRYGLNGKEVQWKMYEVYDAKSKSASVPVPELCAVYAVQVRCKRLDGLGYWSNWSSPAYTVIMDIKVPTRGPEFWRMIDEDTSRKERNVTLLWKPLMKNDSLCSVRKYVVKHHTSRNGTWSEDVGNHTKFTFLWTEQAHSVTVLAVNSIGASSVNFNLTFSWPMSKVNTVQSLSAYPLNSTCVLLSWTLTPSDYYLTYFITEWKILNEDSEIKWLRIPPSVKKYYIHDHFIPIEKYQFSLYPVFMEGVGKPKTINSFTQDDIEKHQNDAGLYVILLIIISSSILLLGTLLISHQRMKKLFWEDVPNPKNCSWAQGLNFQKPETFEHLFIKHTESVIFGPLLLEPETISEDISVDTSWKNKDEMVPTTMVSLLLTTPDLEKGSICIRDQHNSANFSELESTVVTREDEGRRQPSVRYATLLTSSKSSEIEEEQGLINSSVSKCFSSKNSLPKGSFSNSSWEIETQAFFILSDQHPNIILPHLPFSEGLDDLLKLEGNFPEENNGERSVYYLGVTSIKKRESGVFLTDESQVLCPFPAHCLFTDIRILQDSCSHLVENNFNLGTSGQKTFVPYMPQFQICSTQTQKIMETKMCDLTV

SEQ ID NO: 33 - protein, 1162 aa, Rattus rattus
MTCQKFYVVLLHWEFLYVITALNLAYPTSPWRFKLFCAPPSTTDDSFLSPAGGPNNSSSLKGASEALVEAKFNSSGIYVSELSKTIFHCCFGNEQGQNCSALTGNTEGKTLASVVKPLVFRQLGVNWDIECWMKGDLTLFICHMEPLLKNPFKNYDSKVHLLYDLPEVIDDLPLPPLKDSFQTVQCNCSVQECECHVPVPRAKVNYALLMYLEITSAGVSFQSPLMSLQPMLVVKPDPPLGLRMEVTDDGNLNISWDSQTKAPFPLQYQVKYLENSTIVREAAEIVSDTSLLVDSVLPGSSYEVQVRSKRLDGSGVWSDWSLPQVFTTQDVMYFPPKILTSVGSNASFRCIYKNENQTISSKQIVWWMNLAEKIPETQYNTVSDHISKVTFSNLKATRPRGKFAYDAVYCCNEQACHHRYAELYVIDVNINISCETDGYLTKMTCRWSPSTIQSLVGSTVQMRYHRRSLYCPDNPSIRPTSELKNCVLQRDGFYECVFQPIFLLSGYTMWIRINHSLGSLDSPPTCVLPDSVVKPLPPSNVKAEITINTGLLKVSWEKPVFPENNLQFQIRYGLNGKEMQWKTHEVFDAKSKSASLPVSDLCAVYVVQVRCRRLDGLGYWSNWSSPAYTLVMDVKVPMRGPEFWRIMDGDITKKERNVTLLWKPLMKNDSLCSVRRYVVKHRTAHNGTWSQDVGNQTNLTFLWAESAHTVTVLAINSIGASLVNFNLTFSWPMSKVNAVQSLSAYPLSSSCVILSWTLSPDDYSLLYLVIEWKNLNDDDGMKWLRIPSNVNKYYIHDNFIPIEKYQFSLYPVFMEGVGKPKTINGFTKDDIAKQKNDAGLYVIVPIIISSCVLLLGTLLISHQRMKKLFWDDVPNPKNCSWAQGLNFQKPETFEHLFTKHAESVIFGPLLLEPEPISEEISVDTAWKNKDEMVPAAMVSLLLTTPDSTRGSICISDQCNSANFSGAQSTQGTCEDECQSQPSVKYATLVSNVKPVETDEEQGAIHSSVSQCITRKHSPLRQSFSSSSWEIEAQAFLLLSDHPPNVISPQLSFSGLDELLELEGNFPEENHGEKSVYYLGVSSVNKRENDMLLTDEAGVLCPFPAHCLFSDIRILQESCSHFVENNLNLGTSGKNFVPYMPQFQSCSTHSHKIIENKMCDLTV

SEQ ID NO: 34 - protein, 1163 aa, Mus musculus
MMCQKFYVVLLHWAEFLYVIAALNLAYPISPWKFKLFCGPPNTTDDSFLSPAGAPNNASALKGASEAIVEAKFNSSGIYVPELSKTVFHCCFGNEQGQNCSALTDNTEGKTLASVVKASVFRQLGVNWDIECWMKGDLTLFICHMEPLPKNPFKNYDSKVHLLYDLPEVIDDSPLPPLKDSFQTVQCNCSLRGCECHVPVPRAKLNYALLMYLEITSAGVSFQSPLMSLQPMLVVKPDPPLGLHMEVTDDGNLKISWDSQTMAPFPLQYQVKYLENSTIVREAAEIVSATSLLVDSVLPGSSYEVQVRSKRLDGSGVWSDWSSPQVFTTQDVVYFPPKILTSVGSNASFHCIYKNENQIISSKQIVWWRNLAEKIPEIQYSIVSDRVSKVTFSNLKATRPRGKFTYDAVYCCNEQACHHRYAELYVIDVNINISCETDGYLTKMTCRWSPSTIQSLVGSTVQLRYHRRSLYCPDSPSIHPTSEPKNCVLQRDGFYECVFQPIFLLSGYTMWIRINHSLGSLDSPPTCVLPDSVVKPLPPSNVKAEITVNTGLLKVSWEKPVFPENNLQFQIRYGLSGKEIQWKTHEVFDAKSKSASLLVSDLCAVYVVQVRCRRLDGLGYWSNWSSPAYTLVMDVKVPMRGPEFWRKMDGDVTKKERNVTLLWKPLTKNDSLCSVRRYVVKHRTAHNGTWSEDVGNRTNLTFLWTEPAHTVTVLAVNSLGASLVNFNLTFSWPMSKVSAVESLSAYPLSSSCVILSWTLSPDDYSLLYLVIEWKILNEDDGMKWLRIPSNVKKFYIHDNFIPIEKYQFSLYPVFMEGVGKPKIINGFTKDAIDKQQNDAGLYVIVPIIISSCVLLLGTLLISHQRMKKLFWDDVPNPKNCSWAQGLNFQKPETFEHLFTKHAESVIFGPLLLEPEPISEEISVDTAWKNKDEMVPAAMVSLLLTTPDPESSSICISDQCNSANFSGSQSTQVTCEDECQRQPSVKYATLVSNDKLVETDEEQGFIHSPVSNCISSNHSPLRQSFSSSSWETEAQTFFLLSDQQPTMISPQLSFSGLDELLELEGSFPEENHREKSVCYLGVTSVNRRESGVLLTGEAGILCTFPAQCLFSDIRILQERCSHFVENNLSLGTSGENFVPYMPQFQTCSTHSHKIMENKMCDLTV

SEQ ID NO: 35 - protein, 829 aa, Oryctolagus cuniculus
FNLAYPVTPWRFKLSCMPANATHDYFLLPAGISKNTSNSSGHYEAIIEDKFNSSDTYFSNLSQTTFYCCFWSEQDTNCSVRADNIEGKTFVSTVNSLVFQQVGANWDIQCQMKGDLKLFICYMESLLKNPFKNVGLKVHLLYVLPEMLEDSLLVPQKGTFQMVQCNCSVHERCECHVPVPAAKLNYTLLMYFKVTSGGVFLQSPLMSVQLIDAVKPDPPLGLRMEITDKGNLKISWSNPAQVPFPLQYQVKYSENSTTIIREVAEIVSATFLLVDSVLPGSSYKAQVRGRRLDGPGTWSDWSTPQIFVTQDVIYFPPKILTSVGSNVSFHCIYKNEHKIVSSKQIVWWMNLAEKIPQSQYTVVNDRVSKVTFPNLNATKPRGKFTYDAVYCCRDHECHHRYAELYVVDVNINISCETDGYLTKMTCRWSANTIQSLVGSTLQLRYHRSSLYCSDIPSIHPISEPKECHLQRDGFYECIFQPIFLLSGYTMWIRVNHSLGSLDSPPTCVLPDSVVKPLPPSIVKAEITVNIGLLKLSWEKPVFPENNLQFQIRYGLSGNEKQWKVFEVHDSKSKSANLSVPDLCAVYAAQVRCKRLDGLGYWSNWSKPAYTVVKDVKVPVRGPEFWRIIDGDVTKKERNVTLLWKPLMKSDSLCSVSRYVVNHYTSHNGTWSEDVGNHTRFTFIWAEQVHTVTVVAINSIGASSSNFNLTFSWPVSKVNTVQSLSAYPLNSSCVILSWIPLPSDYNLMYFIIEWKNLNEDNEIKWLRIPSSVKKYSIHDNFIPIEKYQFSLYPIFMEGVGKPKIINSFTQDNDEKLQNDAGAAAHHHHHH

SEQ ID NO: 36 - protein, 446 aa, synthetic construct
QVQLVESGGSVVQPGRSLRLSCAASGFTFSTYAMYWVRQTPGKGLEWVAVLYSDGSNKYYIDSVKGRFTISRDTSTNTLYLQMSSLRADDSALYYCARLNWDYWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 376 aa, Mus musculus
VPRDSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 39 - protein, 37 aa, synthetic construct
  SITE 1: note = Lysine is chemically modified by conjugation of the epsilon-amino group of the Lysine side chain with (gamma-Glutamic acid)2-CO-(CH2)18-CO2H
  DISULFID 2..7: note = disulfide linkage (Cys-Cys)
  SITE 37: note = Amidated Residue
KCNTATCATQRLADFLRHSSPNFGAIPSSTNVGSRTY

SEQ ID NO: 40 - protein, 37 aa, synthetic construct
  SITE 1: note = gamma-Glutamic acid
  CROSSLNK 2..7: note = methylene thioacetal linkage (Cys-Cys)
  SITE 11: note = L-ornithine
  SITE 15: note = alpha-methyl-phenylalanine
  SITE 22: note = N-methylated asparagine
  SITE 37: note = Amidated Residue
XCNTATCATGXLAEFLVRSSNNFGPKLPPTEVGSNTY

SEQ ID NO: 41 - protein, 37 aa, synthetic construct; identical to SEQ ID NO: 40
  SITE 1: note = gamma-Glutamic acid
  CROSSLNK 2..7: note = methylene thioacetal linkage (Cys-Cys)
  SITE 11: note = L-ornithine
  SITE 15: note = alpha-methyl-phenylalanine
  SITE 22: note = N-methylated asparagine
  SITE 26: note = Lysine is chemically modified by conjugation of the epsilon-amino group of the Lysine side chain with (gamma-Glutamic acid)2-CO-(CH2)18-CO2H
  SITE 37: note = Amidated Residue

SEQ ID NO: 42 - protein, 37 aa, synthetic construct
  SITE 1: note = gamma-Glutamic acid
  CROSSLNK 2..7: note = methylene thioacetal linkage (Cys-Cys)
  SITE 11: note = L-ornithine
  SITE 15: note = alpha-methyl-phenylalanine
  SITE 22: note = N-methylated aspartic acid
  SITE 37: note = Amidated Residue
XCNTATCATGXLAEFLVRSSNDFGPKLPPTEVGSNTY

SEQ ID NO: 43 - protein, 37 aa, synthetic construct; identical to SEQ ID NO: 42
  SITE 1: note = gamma-Glutamic acid
  CROSSLNK 2..7: note = methylene thioacetal linkage (Cys-Cys)
  SITE 11: note = L-ornithine
  SITE 15: note = alpha-methyl-phenylalanine
  SITE 22: note = N-methylated aspartic acid
  SITE 26: note = Lysine is chemically modified by conjugation of the epsilon-amino group of the Lysine side chain with (gamma-Glutamic acid)2-CO-(CH2)18-CO2H
  SITE 37: note = Amidated Residue

SEQ ID NO: 44 - protein, 37 aa, synthetic construct
  CROSSLNK 2..7: note = methylene thioacetal linkage (Cys-Cys)
  SITE 11: note = L-ornithine
  SITE 15: note = alpha-methyl-phenylalanine
  SITE 22: note = N-methylated aspartic acid
  SITE 37: note = Amidated Residue
KCETATCATGXLAEFLVRSSNDFGPILPPTEVGSNTY

SEQ ID NO: 45 - protein, 37 aa, synthetic construct; identical to SEQ ID NO: 44
  SITE 1: note = Lysine is chemically modified by conjugation of the epsilon-amino group of the Lysine side chain with (gamma-Glutamic acid)2-CO-(CH2)18-CO2H
  SITE 2..7: note = methylene thioacetal linkage (Cys-Cys)
  SITE 11: note = L-ornithine
  SITE 15: note = alpha-methyl-phenylalanine
  SITE 22: note = N-methylated aspartic acid
  SITE 37: note = Amidated Residue

SEQ ID NO: 46 - protein, 37 aa, synthetic construct
  CROSSLNK 2..7: note = methylene thioacetal linkage (Cys-Cys)
  SITE 11: note = L-ornithine
  SITE 12: note = alpha-methyl-leucine
  SITE 37: note = Amidated Residue
KCETATCATGXLAEFLVRSSHNFGPILPPTEVGSNTY

SEQ ID NO: 47 - protein, 37 aa, synthetic construct; identical to SEQ ID NO: 46
  SITE 1: note = Lysine is chemically modified by conjugation of the epsilon-amino group of the Lysine side chain with (gamma-Glutamic acid)2-CO-(CH2)18-CO2H
  CROSSLNK 2..7: note = methylene thioacetal linkage (Cys-Cys)
  SITE 11: note = L-ornithine
  SITE 12: note = alpha-methyl-leucine
  SITE 37: note = Amidated Residue

SEQ ID NO: 48 - protein, 32 aa, synthetic construct
  SITE 1: note = N-terminal acetylated residue
  SITE 14: note = 2-Aminoisobutyric Acid
  SITE 32: note = Amidated Residue
ASHLSTAVLGKLSXELHKLEDYPRTDVGAESP

SEQ ID NO: 49 - protein, 32 aa, synthetic construct; identical to SEQ ID NO: 48
  SITE 1: note = N-terminal acetylated residue
  SITE 11: note = Lysine is chemically modified by conjugation of the epsilon-amino group of the Lysine side chain with (2-[2-(2-Amino-ethoxy)-ethoxy]-acetyl)2-gamma-Glutamic acid-CO-(CH2)18-CO2H
  SITE 14: note = 2-Aminoisobutyric Acid
  SITE 32: note = Amidated Residue

SEQ ID NO: 50 - protein, 37 aa, synthetic construct
  CROSSLNK 2..7: note = methylene thioacetal linkage (Cys-Cys)
  SITE 37: note = Amidated Residue
KCNTATCATQRLANFLVHSSNNFGPILPPTNVGSNTY

SEQ ID NO: 51 - protein, 37 aa, synthetic construct
  SITE 1: note = Lysine is chemically modified by conjugation of the epsilon-amino group of the Lysine side chain with CO2H-(CH2)18-CO-(gamma-Glutamic acid)
  SITE 37: note = Amidated Residue
KCNTATCATQRLAEFLRHSSNNFGPILPPTNVGSNTP

SEQ ID NO: 52 - protein, 31 aa, synthetic construct
  SITE 2: note = 2-Aminoisobutyric Acid
  SITE 20: note = Lysine is chemically modified by conjugation of the epsilon-amino group of the Lysine side chain with (2-[2-(2-Amino-ethoxy)-ethoxy]-acetyl)2-gamma-Glutamic acid-CO-(CH2)16-CO2H
HXEGTFTSDVSSYLEGQAAKEFIAWLVRGRG

SEQ ID NO: 53 - protein, 39 aa, synthetic construct
  SITE 2: note = 2-Aminoisobutyric Acid
  SITE 13: note = 2-Aminoisobutyric Acid
  SITE 20: note = Lysine is chemically modified by conjugation of the epsilon-amino group of the Lysine side chain with (2-[2-(2-Amino-ethoxy)-ethoxy]-acetyl)2-gamma-Glutamic acid-CO-(CH2)18-CO2H
  SITE 22: note = 3-(1-naphthyl)-L-alanine
  SITE 39: note = Amidated Residue
YXEGTFTSDYSIXLDKIAQKAXVQWLIAGGPSSGAPPPS

SEQ ID NO: 54 - protein, 39 aa, synthetic construct
  SITE 2: note = 2-Aminoisobutyric Acid
  SITE 13: note = 2-Aminoisobutyric Acid
  SITE 20: note = Lysine is chemically modified by conjugation of the epsilon-amino group of the Lysine side chain with (2-[2-(2-Amino-ethoxy)-ethoxy]-acetyl)2-gamma-Glutamic acid-CO-(CH2)18-CO2H
  SITE 39: note = Amidated Residue
YXEGTFTSDYSIXLDKIAQKAFVQWLIAGGPSSGAPPPS

SEQ ID NO: 55 - protein, 39 aa, synthetic construct
  SITE 2: note = 2-Aminoisobutyric Acid
  SITE 17: note = Lysine is chemically modified by conjugation of the epsilon-amino group of the Lysine side chain with (2-[2-(2-Amino-ethoxy)-ethoxy]-acetyl)2-gamma-Glutamic acid-CO-(CH2)18-CO2H
  SITE 20: note = 2-Aminoisobutyric Acid
  SITE 39: note = Amidated Residue
YXQGTFTSDYSILLDKKAQXAFIEYLLEGGPSSGAPPPS

SEQ ID NO: 56 - protein, 39 aa, synthetic construct; identical to SEQ ID NO: 55
  SITE 2: note = 2-Aminoisobutyric Acid
  SITE 13: note = alpha-methyl-leucine
  SITE 17: note = Lysine is chemically modified by conjugation of the epsilon-amino group of the Lysine side chain with (2-[2-(2-Amino-ethoxy)-ethoxy]-acetyl)-gamma-Glutamic acid-CO-(CH2)18-CO2H
  SITE 39: note = Amidated Residue

SEQ ID NO: 57 - protein, 39 aa, synthetic construct
  SITE 2: note = 2-Aminoisobutyric Acid
  SITE 16: note = 2-Aminoisobutyric Acid
  SITE 20: note = Lysine is chemically modified by conjugation of the epsilon-amino group of the Lysine side chain with (2-[2-(2-Amino-ethoxy)-ethoxy]-acetyl)2-(gamma-Glutamic acid)2-CO-(CH2)16-CO2H
  SITE 39: note = Amidated Residue
YXQGTFTSDYSKYLDXKKAKEFVEWLLETGPSSGAPPPS

SEQ ID NO: 58 - protein, 823 aa, Macaca fascicularis
FNLSYPITPWRFKLSCMPPNSTYDYFLLPAGLSKNTSNLNGHYETAVEFNSSDTHFSNLSKTTFHCCFRSEQDRNCSLCADNIEGKTFVSTVNSSVFQQMGANWNIQCWLKGDLKLFICYVESLFKNPFKNYKHKVHLLYVLPEVLEDSPLVPQKGSFQMVHCNCSVHERCECLVPVPTAKLNDTLLMCLKITSGGVIFQSPLMSVQPINMVKPDPPLGLRMEITDDGNLKISWSSPPLVPFPLQYEVKYSENSTTVIREADKIVSATSLLVDGILPGSSYEVQVRGKRLDGPGIWSDWSTPHVFTTQDVIYFPPKILTSVGSNVSFHCIYKNENKIVSSKKIVWWMNLAEKIPQSQYDVVSDHVSKVTFFNLNETKPRGKFTYDAVYCCNEHECHHRYAELYVIDVNINISCETDGHLTKMTCRWSTNTIQSLAGSTLQLRYRRSSLYCFDIPSIHPISKPKDCYLQSDGFYECVFQPIFLLSGYTMWIRINHPLGSLDSPPTCVLPDSVVKPLPPSSVKAEIIKNIGLLKISWEKPVFPENNLQFQIRYGLSGKEIQWKMYDVYDAKSKSVSLPVPDFCAVYAVQVRCKRSDGLGLWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVILSWILSPSDYKLMYFIIEWKNLNEDGEIKWLRISSSVKKYYIHDHFIPIEKYQFSLYPIFMEGVGKPKIINSFTQDNTEKHQNDGHHHHHH

SEQ ID NO: 59 - protein, 824 aa, Canis lupus
  source 1..824: note = dingo
FNLAYPITPWRFKLSCMPPNTTYDFLLPAGISRNTSNLNEHYEAVVEAKLNSSSTYISNLSSKTTFHCCFWSKEDKNCSVHADNMEGKAFVSTVSSLVFQQIGANWNIQCWMKEDLKLFICYMESLFKNPFKTYDLKVHLLYVLPEVLEESPPVPQKGGFQIVPCNCSVHDSCECHVPVPTAELNHTLLMYLKITLGGINFQSPLMSVKPINVVKPDPPLGLHMEITDTGNLKISWSSPTLVPFQLQYQVRYSENSSTNVRKANEIVSATSLLIDSVLPGSSYEVQVRGKKLDGPGIWGDWSTPLIFITQDVIYFPPKILTSVGSNVSFHCIYKSENKIVSSKKIVWWLNLAEKIPQSQYNMVGDRVSKVTFPNLNATKPRGKFTYDAVYCCKEQECHHRYAELYVIDVNINISCETDGYLTKMTCRWSTNAIQSLEGSTLQLRYHRSSLYCSDVPSIHPISEPKDCHLRRDGFYECIFQPIFLLSGYTMWIKINHSLGSLDSSPTCVVPDSVVKPLPPSSVKAEITVKIGLLKISWEKPVFPENNLKFQIRYGLNGKEVQWKIYEVYDTKLKSTSLPVPDLCAVYAVQVRCKRLDGLGYWSNWSTPAYTVVTDVKVPTRGPEFWRMIDEDTSRKERNVTLLWKPLMKNDSLCSVRKYVVKHHTSRNGTWSEDVGNHTKFTFLWTEQAHSVTVLAVNSIGASSVNFNLTFSWPMSKVNTVQSLSAYPLNSTCVLLSWTLTPSDYYLTYFITEWKILNEDSEIKWLRIPPSVKKYYIHDHFIPIEKYQFSLYPVFMEGVGKPKTINSFTQDDIEKHQNDHHHHHH

SEQ ID NO: 60 - protein, 827 aa, Rattus rattus
LNLAYPTSPWRFKLFCAPPSTTDDSFLSPAGVPNNTSSLKGASEALVEAKFNSTGIYVSELSKTIFHCCFGNEQGQNCSALTGNTEGKTLASVVKPLVFRQLGVNWDIECWMKGDLTLFICHMEPLLKNPFKNYDSKVHLLYDLPEVIDDLPLPPLKDSFQTVQCNCSVRECECHVPVPRAKVNYALLMYLEITSAGVSFQSPLMSLQPMLVVKPDPPLGLRMEVTDDGNLKISWDSQTKAPFPLQYQVKYLENSTIVREAAEIVSDTSLLVDSVLPGSSYEVQVRSKRLDGSGVWSDWSLPQLFTTQDVMYFPPKILTSVGSNASFCCIYKNENQTISSKQIVWWMNLAEKIPETQYNTVSDHISKVTFSNLKATRPRGKFTYDAVYCCNEQACHHRYAELYVIDVNINISCETDGYLTKMTCRWSPSTIQSLVGSTVQLRYHRRSLYCPDNPSIRPTSELKNCVLQTDGFYECVFQPIFLLSGYTMWIRINHSLGSLDSPPTCVLPDSVVKPLPPSNVKAEITINTGLLKVSWEKPVFPENNLQFQIRYGLNGKEIQWKTHEVFDAKSKSASLPVSDLCAVYVVQVRCRRLDGLGYWSNWSSPAYTLVMDVKVPMRGPEFWRIMDGDITKKERNVTLLWKPLMKNDSLCSVRRYVVKHRTAHNGTWSQDVGNQTNLTFLWAESAHTVTVLAINSIGASLVNFNLTFSWPMSKVNAVQSLSAYPLSSSCVILSWTLSPNDYSLLYLVIEWKNLNDDDGMKWLRIPSNVNKYYIHDNFIPIEKYQFSLYPVFMEGVGKPKIINGFTKDDIAKQQNDAGAAAHHHHHH

SEQ ID NO: 61 - protein, 830 aa, Mus musculus
LNLAYPISPWKFKLFCGPPNTTDDSFLSPAGAPNNASALKGASEAIVEAKFNSSGIYVPELSKTVFHCCFGNEQGQNCSALTDNTEGKTLASVVKASVFRQLGVNWDIECWMKGDLTLFICHMEPLPKNPFKNYDSKVHLLYDLPEVIDDSPLPPLKDSFQTVQCNCSLRGCECHVPVPRAKLNYALLMYLEITSAGVSFQSPLMSLQPMLVVKPDPPLGLHMEVTDDGNLKISWDSQTMAPFPLQYQVKYLENSTIVREAAEIVSATSLLVDSVLPGSSYEVQVRSKRLDGSGVWSDWSSPQVFTTQDVVYFPPKILTSVGSNASFHCIYKNENQIISSKQIVWWRNLAEKIPEIQYSIVSDRVSKVTFSNLKATRPRGKFTYDAVYCCNEQACHHRYAELYVIDVNINISCETDGYLTKMTCRWSPSTIQSLVGSTVQLRYHRRSLYCPDSPSIHPTSEPKNCVLQRDGFYECVFQPIFLLSGYTMWIRINHSLGSLDSPPTCVLPDSVVKPLPPSNVKAEITVNTGLLKVSWEKPVFPENNLQFQIRYGLSGKEIQWKTHEVFDAKSKSASLLVSDLCAVYVVQVRCRRLDGLGYWSNWSSPAYTLVMDVKVPMRGPEFWRKMDGDVTKKERNVTLLWKPLTKNDSLCSVRRYVVKHRTAHNGTWSEDVGNRTNLTFLWTEPAHTVTVLAVNSLGASLVNFNLTFSWPMSKVSAVESLSAYPLSSSCVILSWTLSPDDYSLLYLVIEWKILNEDDGMKWLRIPSNVKKFYIHDNFIPIEKYQFSLYPVFMEGVGKPKIINGFTKDAIDKQQNDAGAAAHHHHHHSGS